(19) United States
(12) Patent Application Publication
(10) Pub. No.: US 2021/0093567 A1
(43) Pub. Date: Apr. 1, 2021

(54) THERAPEUTIC EXTRACELLULAR VESICLES

(71) Applicant: Ohio State Innovation Foundation, Columbus, OH (US)

(72) Inventors: L. James Lee, Columbus, OH (US); Junfeng Shi, Columbus, OH (US); Zhaogang Yang, Columbus, OH (US)

(21) Appl. No.: 16/986,954

(22) Filed: Aug. 6, 2020

Related U.S. Application Data
(60) Provisional application No. 62/947,228, filed on Dec. 12, 2019.
(60) Provisional application No. 62/883,319, filed on Aug. 6, 2019.

(51) Int. Cl. A61K 9/127; C12N 13/00; C07K 14/705; A61K 35/33; A61K 35/15

(52) U.S. Cl. CPC A61K 9/1277; C12N 13/00; A61K 35/15; A61K 35/33; C07K 14/70503

(57) ABSTRACT

Described herein are compositions of therapeutic extracellular vesicles, and methods and systems of producing the therapeutic extracellular vesicles. Also described herein are methods of treating a disease with the therapeutic extracellular vesicles.

### CROSS REFERENCE

This application claims the benefit of U.S. Provisional Application Ser. No. 62/883,319 filed on Aug. 6, 2019 and U.S. Provisional Application Ser. No. 62/947,228 filed on Dec. 12, 2019, the entireties of which are hereby incorporated by reference herein.

### INCORPORATION BY REFERENCE

All publications, patents, and patent applications mentioned in this specification are herein incorporated by reference in their entireties to the same extent as if each individual publication, patent, or patent application was specifically and individually indicated to be incorporated by reference.

### BACKGROUND

Extracellular vesicles are secreted by a wide variety of cell types. In general, extracellular vesicles such as exosomes, microvesicles, and apoptotic bodies are membrane-bound and can be loaded with a therapeutic cargo. Exosomes are a type of extracellular vesicle that are secreted by most eukaryotic cells. Exosome biogenesis may begin when endosomal invaginations pinch off into the multivesicular body, forming intraluminal vesicles. If the multivesicular body fuses with the plasma membrane of the cell, the intraluminal vesicles may be released as exosomes. Microvesicles are another type of extracellular vesicles that are outward budded from cell surface membrane. Apoptotic bodies, on the other hand, are extracellular vesicles that are formed from dead cell debris. Exosomes, microvesicles, and apoptotic bodies can be released in vivo or in vitro, such as in cell-culture.

Delivery of therapeutic genetic material can be useful for treating disease. Extracellular vesicles have been examined as carriers for therapeutic nucleic acids. However, most current methods of producing extracellular vesicles and encapsulation of therapeutic nucleic acids within the extracellular vesicles have several drawbacks. First, the yield of producing extracellular vesicles incorporating the therapeutic nucleic acids is generally low, often because low numbers of extracellular vesicles are produced or because a low number of copies of the therapeutic nucleic acid is encapsulated in the extracellular vesicles. For example, when some modes of transfection are employed, messenger RNA (mRNA) is generally too large to be effectively encapsulated by extracellular vesicles. Other issues stemming from the currently-available methods include fragmentation and degradation of the nucleic acids encapsulated by the extracellular vesicles. Finally, directing the extracellular vesicles to an in vivo target remains a challenge, as the majority of the extracellular vesicles in circulation accumulate and are metabolized in the liver, spleen, and kidney.

Therefore, there is a need for a pharmaceutical composition comprising extracellular vesicles that can effectively deliver a sufficient quantity of therapeutic nucleic acids to a target cell, tissue or organ. There also is a need for methods and systems of producing a pharmaceutical composition comprising extracellular vesicles in order to deliver a sufficient quantity of high quality therapeutic nucleic acids to a target to treat a disease in a subject.

### SUMMARY

Described herein, in some aspects, is a method of producing an extracellular vesicle, said method comprising: nanoelectroporating an extracellular vesicle donor cell with at least one polynucleotide, wherein said at least one polynucleotide encodes a targeting polypeptide that comprises: (i) an adapter polypeptide comprising a transmembrane domain and an extracellular domain; and (ii) a heterologous targeting domain that is covalently linked to said extracellular domain of said adapter polypeptide; incubating said extracellular vesicle donor cell under conditions such that (i) said targeting polypeptide is expressed in said extracellular vesicle donor cell and (ii) said targeting polypeptide is incorporated into an extracellular vesicle released from said extracellular vesicle donor cell; and collecting said at least one extracellular vesicle released from said extracellular vesicle donor cell, wherein said at least one extracellular vesicle comprises said targeting polypeptide. In some embodiments, said heterologous targeting domain is covalently linked to a N terminus of said extracellular domain of said adapter polypeptide. In some embodiments, said heterologous targeting domain is covalently linked to a C terminus of said extracellular domain of said adapter polypeptide. In some embodiments, said transmembrane domain of said adapter polypeptide is at least 70% identical to a transmembrane domain of a CD47 polypeptide or said extracellular domain of said adapter polypeptide is at least 70% identical to an extracellular domain of a CD47 polypeptide. In some embodiments, said transmembrane domain of said adapter polypeptide is at least 80% identical, at least 90% identical, at least 95% identical, at least 99% identical, or 100% identical to a transmembrane domain of a CD47 polypeptide or said extracellular domain of said adapter polypeptide is at least 80% identical, at least 90% identical, at least 95% identical, at least 99% identical, or 100% identical to an extracellular domain of a CD47 polypeptide. In some embodiments, said adapter polypeptide is selected from the group consisting of CD63, CD81, CD82, CD47, CD315, heterotrimeric G protein, MHC class I, integrins, transferrin receptor (TFR2), LAMP1/2, heparan sulfate proteoglycans, EMMPRIN, ADAM10, GPI-anchored 5′nucleotidase, CD73, complement-binding protein CD55 and CD59, sonic hedgehog (SHH), TSPAN8, CD37, CD53, CD9, PECAM1, ERBB2, EPCAM, CD90, CD45, CD41, CD42a, Glycophorin A, CD14, MHC class II, CD3, Acetylcholinesterase/AChE-S, AChE-E, amyloid beta A4/APP, PTGFRN, and multidrug resistance-associated protein. In some embodiments, said adapter polypeptide is at least 70% identical to a polypeptide selected from the group consisting of: CD63, CD81, CD82, CD47, CD315, heterotrimeric G protein, MHC class I, integrins, transferrin receptor (TFR2), LAMP1/2, heparan sulfate proteoglycans, EMMPRIN, ADAM10, GPI-anchored 5′nucleotidase, CD73, complement-binding protein CD55 and CD59, sonic hedgehog (SHH), TSPAN8, CD37, CD53, CD9, PECAM1, ERBB2, EPCAM, CD90, CD45, CD41, CD42a, Glycophorin A, CD14, MHC class II, CD3, Acetylcholinesterase/AChE-S, AChE-E, amyloid beta A4/APP, PTGFRN, and multidrug resistance-associated protein. In some embodiments, said adapter polypeptide is at least 70% identical to CD47. In some embodiments, said adapter polypeptide is at least 80% identical, at least 90% identical, at least 95% identical, at least 99% identical, or 100% identical to CD47. In some embodiments, said heterologous targeting domain comprises a tumor targeting domain. In some embodiments, said tumor targeting domain is a CDX peptide. In some embodiments, said tumor targeting domain is a CREKA peptide. In some embodiments, said method further comprising: nanoelectroporating a polynucleotide into said extracellular donor cell, wherein said polynucleotide encodes a ribonucleic acid (RNA) therapeutic. In some embodiments, said ribonucleic acid (RNA) therapeutic is incorporated into extracellular vesicles released from said extracellular vesicle donor cell and said method further comprises collecting said extracellular vesicles released from said extracellular vesicle donor cell. In some embodiments, said ribonucleic acid (RNA) therapeutic is a messenger RNA (mRNA) therapeutic. In some embodiments, said ribonucleic acid (RNA) therapeutic is a non-coding RNA, a microRNA, a shRNA, a siRNA, or a combination thereof. In some embodiments, said RNA therapeutic is a cancer drug. In some embodiments, said extracellular vesicle comprises said RNA therapeutic in a fully intact or substantially intact form. In some embodiments, said RNA therapeutic is fully intact or substantially intact messenger RNA. In some embodiments, said RNA therapeutic comprises at least 5 copies of fully intact messenger RNA. In some embodiments, following said nanoelectroporation, on average, each extracellular vesicle released by said extracellular vesicle donor cell comprises at least one copy of said RNA therapeutic. In some embodiments, following said nanoelectroporation, on average, each extracellular vesicle released by said extracellular vesicle donor cell comprises at least one fully intact or substantially intact copy of said RNA therapeutic. In some embodiments, prior to said nanoelectroporation, said extracellular vesicle donor cell is a primary cell or a genetically-unmodified cell. In some embodiments, said extracellular vesicle donor cell is selected from the group consisting of: mouse embryonic fibroblasts (MEF), human embryonic fibroblasts (HEF), dendritic cells, mesenchymal stem cells, bone marrow-derived dendritic cells, bone marrow derived stromal cells, adipose stromal cells, endothelial cells, and immune cells. In some embodiments, said extracellular vesicle donor cell is not a neutrophil. In some embodiments, said extracellular vesicle is an exosome, a microvesicle, or an apoptotic body. In some embodiments, said extracellular vesicle is an exosome. In some embodiments, said polynucleotide is nanoelectroporated into said extracellular vesicle donor cell via a nanochannel located on a biochip. In some embodiments, said nanochannel comprises a diameter from 1 nanometer to 1000 nanometers. In some embodiments, said nanochannel comprises a diameter from 200 nanometers to 800 nanometers. In some embodiments, said nanochannel comprises a diameter of about 500 nanometers. In some embodiments, said biochip comprises an array of nanochannels comprising a spacing between nanochannels from 1 micrometer to 100 micrometers. In some embodiments, said nanoelectroporation comprises an electric field. In some embodiments, said electric field has an electric field strength from 1 volt/mm to 1000 volt/mm In some embodiments, said electric field comprises a plurality of pulses with pulse durations from 0.1 milliseconds/pulse to 100 millisecond/pulse. In some embodiments, the tumor targeting domain is on a N-terminus of the tumor targeting polypeptide. In some embodiments, the tumor targeting domain is on a C-terminus of the tumor targeting polypeptide.

Described herein, in some aspects, is a method of producing an extracellular vesicle, said method comprising: nanoelectroporating a primary cell with at least one heterologous deoxyribonucleic acid (DNA) polynucleotide, thereby obtaining a primary cell comprising said heterologous DNA polynucleotide, wherein said heterologous DNA polynucleotide encodes a therapeutic ribonucleic acid (RNA) polynucleotide; incubating said primary cell comprising said heterologous DNA polynucleotide under conditions to enable transcription of said heterologous DNA polynucleotide, thereby producing said therapeutic ribonucleic acid (RNA) polynucleotide, wherein said therapeutic ribonucleic acid (RNA) polynucleotide is incorporated into extracellular vesicles released from said primary cell; and collecting said extracellular vesicles released from said primary cell, wherein said extracellular vesicles released from said primary cell comprise, on average, at least one copy of said therapeutic ribonucleic acid (RNA) polynucleotide. In some cases, said extracellular vesicles released from said primary cell comprise, on average, at least one copy of said therapeutic ribonucleic acid (RNA) polynucleotide, for every 5, 10, 20, 50, 100, 500 or 1000 extracellular vesicle released from said primary cell. In some cases, said extracellular vesicles released from said primary cell comprise, on average, at least 2, 5, 10, 25 or 50 copies of said therapeutic ribonucleic acid (RNA). In some embodiments, prior to said nanoelectroporation, said primary cell is a genetically-unmodified primary cell. In some embodiments, said primary cell is selected from the group consisting of: mesenchymal stem cells, bone marrow-derived dendritic cells, bone marrow derived stromal cells, adipose stromal cells, endothelial cells, and immune cells. In some embodiments, said primary cell is not a neutrophil. In some embodiments, said extracellular vesicle is an exosome, a microvesicle, or an apoptotic body. In some embodiments, said extracellular vesicle is an exosome.

Described herein, in some aspects, is a composition comprising an extracellular vesicle, said extracellular vesicle comprising: an adapter polypeptide, wherein said adapter polypeptide comprises an extracellular domain, wherein said adapter polypeptide comprises a polypeptide sequence that is at least 70% identical to one of the following polypeptides: a CD47 extracellular domain, a CD47 transmembrane domain, CD63, CD81, CD82, CD47, CD315, heterotrimeric G protein, MHC class I, integrins, transferrin receptor (TFR2), LAMP1/2, heparan sulfate proteoglycans, EMMPRIN, ADAM10, GPI-anchored 5′nucleotidase, CD73, complement-binding protein CD55 and CD59, sonic hedgehog (SHH), TSPAN8, CD37, CD53, CD9, PECAM1, ERBB2, EPCAM, CD90, CD45, CD41, CD42a, Glycophorin A, CD14, MHC class II, CD3, Acetylcholinesterase/AChE-S, AChE-E, amyloid beta A4/APP, PTGFRN, and multidrug resistance-associated protein; and a heterologous targeting polypeptide covalently attached to said extracellular domain of said adapter polypeptide, wherein said targeting polypeptide specifically binds to a cellular target. In some embodiments, said adapter polypeptide comprises a transmembrane domain that is at least 70% identical to a transmembrane domain of a CD47 polypeptide or an extracellular domain that is at least 70% identical to an extracellular domain of a CD47 polypeptide. In some embodiments, said heterologous targeting polypeptide is covalently linked to a N terminus of an extracellular domain of said adapter polypeptide. In some embodiments, said heterologous targeting polypeptide is covalently linked to a C terminus of an extracellular domain of said adapter polypeptide. In some embodiments, said adapter polypeptide comprises CD47. In some embodiments, said heterologous targeting polypeptide comprises a targeting domain that binds a cell-surface marker associated with a diseased cell. In some embodiments, said targeting domain is a tumor targeting domain. In some embodiments, said tumor targeting domain is a CDX peptide. In some embodiments, said tumor targeting domain is a CREKA peptide. In some embodiments, said extracellular vesicle comprises at least one copy of ribonucleic acid (RNA) therapeutic. In some embodiments, said ribonucleic acid (RNA) therapeutic is a messenger RNA (mRNA) therapeutic. In some embodiments, said ribonucleic acid (RNA) therapeutic is a non-coding RNA, a microRNA, a shRNA, a siRNA, or a combination thereof. In some embodiments, said RNA therapeutic is a cancer drug. In some embodiments, said RNA therapeutic is a fully intact or substantially intact form. In some embodiments, said RNA therapeutic is fully intact or substantially intact messenger RNA. In some embodiments, said RNA therapeutic comprises at least 5 copies of fully intact or substantially intact messenger RNA. In some embodiments, said extracellular vesicle is an exosome, a microvesicle, or an apoptotic body. In some embodiments, said extracellular vesicle is an exosome. In some embodiments, the tumor targeting domain is on a N-terminus of the tumor targeting polypeptide. In some embodiments, the tumor targeting domain is on a C-terminus of the tumor targeting polypeptide.

Described herein, in some aspects, is a method for treating a tumor in a subject, said method comprising systemically administering at least one extracellular vesicle comprising a therapeutic to the subject, wherein said at least one extracellular vesicle comprising said therapeutic is obtained by: nanoelectroporating an extracellular vesicle donor cell with at least one polynucleotide, wherein said at least one polynucleotide encodes a targeting polypeptide that comprises: (i) an adapter polypeptide comprising a transmembrane domain and an extracellular domain; and (ii) a heterologous targeting domain that is covalently linked to said extracellular domain of said adapter polypeptide, and wherein said at least one polynucleotide encodes a ribonucleic acid (RNA) therapeutic; incubating said extracellular vesicle donor cell under conditions such that (i) said targeting polypeptide is expressed in said extracellular vesicle donor cell and (ii) said targeting polypeptide is incorporated into an extracellular vesicle released from said extracellular vesicle donor cell; and collecting said at least one extracellular vesicle released from said extracellular vesicle donor cell, wherein said at least one extracellular vesicle comprises said targeting polypeptide, wherein accumulation of the at least one extracellular vesicle at the tumor is higher compared to accumulation of an extracellular vesicle lacking the heterologous targeting domain. In some embodiments, said heterologous targeting domain is covalently linked to a N terminus of said extracellular domain of said adapter polypeptide. In some embodiments, said heterologous targeting domain is covalently linked to a C terminus of said extracellular domain of said adapter polypeptide. In some embodiments, said transmembrane domain of said adapter polypeptide is at least 70% identical to a transmembrane domain of a CD47 polypeptide or said extracellular domain of said adapter polypeptide is at least 70% identical to an extracellular domain of a CD47 polypeptide. In some embodiments, said adapter polypeptide is selected from the group consisting of CD63, CD81, CD82, CD47, CD315, heterotrimeric G protein, MHC class I, integrins, transferrin receptor (TFR2), LAMP1/2, heparan sulfate proteoglycans, EMMPRIN, ADAM10, GPI-anchored 5′nucleotidase, CD73, complement-binding protein CD55 and CD59, sonic hedgehog (SHH), TSPAN8, CD37, CD53, CD9, PECAM1, ERBB2, EPCAM, CD90, CD45, CD41, CD42a, Glycophorin A, CD14, MHC class II, CD3, Acetylcholinesterase/AChE-S, AChE-E, amyloid beta A4/APP, PTGFRN, and multidrug resistance-associated protein. In some embodiments, said adapter polypeptide is at least 70% identical to a polypeptide selected from the group consisting of: CD63, CD81, CD82, CD47, CD315, heterotrimeric G protein, MHC class I, integrins, transferrin receptor (TFR2), LAMP1/2, heparan sulfate proteoglycans, EMMPRIN, ADAM10, GPI-anchored 5′nucleotidase, CD73, complement-binding protein CD55 and CD59, sonic hedgehog (SHH), TSPAN8, CD37, CD53, CD9, PECAM1, ERBB2, EPCAM, CD90, CD45, CD41, CD42a, Glycophorin A, CD14, MHC class II, CD3, Acetylcholinesterase/AChE-S, AChE-E, amyloid beta A4/APP, PTGFRN, and multidrug resistance-associated protein. In some embodiments, said adapter polypeptide is at least 70% identical to CD47. In some embodiments, said heterologous targeting domain comprises a tumor targeting domain. In some embodiments, said tumor targeting domain is a CDX peptide. In some embodiments, said tumor targeting domain is a CREKA peptide. In some embodiments, said ribonucleic acid (RNA) therapeutic is incorporated into extracellular vesicles released from said extracellular vesicle donor cell and said method further comprises collecting said extracellular vesicles released from said extracellular vesicle donor cell. In some embodiments, said ribonucleic acid (RNA) therapeutic is a messenger RNA (mRNA) therapeutic. In some embodiments, said ribonucleic acid (RNA) therapeutic is a non-coding RNA, a microRNA, a shRNA, a siRNA, or a combination thereof. In some embodiments, said RNA therapeutic is a cancer drug. In some embodiments, said extracellular vesicle comprises said RNA therapeutic in a fully intact or substantially intact form. In some embodiments, said RNA therapeutic is fully intact or substantially intact messenger RNA. In some embodiments, said RNA therapeutic comprises at least 5 copies of fully intact messenger RNA. In some embodiments, following said nanoelectroporation, on average, each extracellular vesicle released by said extracellular vesicle donor cell comprises at least one copy of said RNA therapeutic. In some embodiments, following said nanoelectroporation, on average, each extracellular vesicle released by said extracellular vesicle donor cell comprises at least one fully intact or substantially intact copy of said RNA therapeutic. In some embodiments, prior to said nanoelectroporation, said extracellular vesicle donor cell is a primary cell or a genetically-unmodified cell. In some embodiments, said extracellular vesicle donor cell is selected from the group consisting of: mouse embryonic fibroblasts (MEF), human embryonic fibroblasts (HEF), dendritic cells, mesenchymal stem cells, bone marrow-derived dendritic cells, bone marrow derived stromal cells, adipose stromal cells, endothelial cells, and immune cells. In some embodiments, said extracellular vesicle donor cell is not a neutrophil. In some embodiments, said extracellular vesicle is an exosome, a microvesicle, or an apoptotic body. In some embodiments, said extracellular vesicle is an exosome. In some embodiments, said polynucleotide is nanoelectroporated into said extracellular vesicle donor cell via a nanochannel located on a biochip. In some embodiments, said nanochannel comprises a diameter from 1 nanometer to 1000 nanometers. In some embodiments, said nanochannel comprises a diameter from 200 nanometers to 800 nanometers. In some embodiments, said nanochannel comprises a diameter of about 500 nanometers. In some embodiments, said biochip comprises an array of nanochannels comprising a spacing between nanochannels from 1 micrometer to 100 micrometers. In some embodiments, said nanoelectroporation comprises an electric field. In some embodiments, said electric field has an electric field strength from 1 volt/mm to 1000 volt/mm In some embodiments, said electric field comprises a plurality of pulses with pulse durations from 0.1 milliseconds/pulse to 100 millisecond/pulse. In some embodiments, the tumor targeting domain is on a N-terminus of the tumor targeting polypeptide. In some embodiments, the tumor targeting domain is on a C-terminus of the tumor targeting polypeptide. In some embodiments, said tumor is cancer. In some embodiments, said cancer is glioma.

Described herein, in some aspects, is a method for treating muscular dystrophy in a subject, said method comprising systemically administering at least one extracellular vesicle comprising a therapeutic to the subject, wherein said at least one extracellular vesicle comprising said therapeutic is obtained by: nanoelectroporating an extracellular vesicle donor cell with at least one polynucleotide, wherein said at least one polynucleotide encodes a targeting polypeptide that comprises: (i) an adapter polypeptide comprising a transmembrane domain and an extracellular domain; and (ii) a heterologous targeting domain that is covalently linked to said extracellular domain of said adapter polypeptide, and wherein said at least one polynucleotide encodes a ribonucleic acid (RNA) therapeutic; incubating said extracellular vesicle donor cell under conditions such that (i) said targeting polypeptide is expressed in said extracellular vesicle donor cell and (ii) said targeting polypeptide is incorporated into an extracellular vesicle released from said extracellular vesicle donor cell; and collecting said at least one extracellular vesicle released from said extracellular vesicle donor cell, wherein said at least one extracellular vesicle comprises said targeting polypeptide, wherein accumulation of the at least one extracellular vesicle at the tumor is higher compared to accumulation of an extracellular vesicle lacking the heterologous targeting domain. In some embodiments, said heterologous targeting domain is covalently linked to a N terminus of said extracellular domain of said adapter polypeptide. In some embodiments, said heterologous targeting domain is covalently linked to a C terminus of said extracellular domain of said adapter polypeptide. In some embodiments, said transmembrane domain of said adapter polypeptide is at least 70% identical to a transmembrane domain of a CD47 polypeptide or said extracellular domain of said adapter polypeptide is at least 70% identical to an extracellular domain of a CD47 polypeptide. In some embodiments, said adapter polypeptide is selected from the group consisting of CD63, CD81, CD82, CD47, CD315, heterotrimeric G protein, MHC class I, integrins, transferrin receptor (TFR2), LAMP1/2, heparan sulfate proteoglycans, EMMPRIN, ADAM10, GPI-anchored 5′nucleotidase, CD73, complement-binding protein CD55 and CD59, sonic hedgehog (SHH), TSPAN8, CD37, CD53, CD9, PECAM1, ERBB2, EPCAM, CD90, CD45, CD41, CD42a, Glycophorin A, CD14, MHC class II, CD3, Acetylcholinesterase/AChE-S, AChE-E, amyloid beta A4/APP, PTGFRN, and multidrug resistance-associated protein. In some embodiments, said adapter polypeptide is at least 70% identical to a polypeptide selected from the group consisting of: CD63, CD81, CD82, CD47, CD315, heterotrimeric G protein, MHC class I, integrins, transferrin receptor (TFR2), LAMP1/2, heparan sulfate proteoglycans, EMMPRIN, ADAM10, GPI-anchored 5′nucleotidase, CD73, complement-binding protein CD55 and CD59, sonic hedgehog (SHH), TSPAN8, CD37, CD53, CD9, PECAM1, ERBB2, EPCAM, CD90, CD45, CD41, CD42a, Glycophorin A, CD14, MHC class II, CD3, Acetylcholinesterase/AChE-S, AChE-E, amyloid beta A4/APP, PTGFRN, and multidrug resistance-associated protein. In some embodiments, said adapter polypeptide is at least 70% identical to CD47. In some embodiments, said heterologous targeting domain comprises a tumor targeting domain. In some embodiments, said tumor targeting domain is a CDX peptide. In some embodiments, said tumor targeting domain is a CREKA peptide. In some embodiments, said ribonucleic acid (RNA) therapeutic is incorporated into extracellular vesicles released from said extracellular vesicle donor cell and said method further comprises collecting said extracellular vesicles released from said extracellular vesicle donor cell. In some embodiments, said ribonucleic acid (RNA) therapeutic is a messenger RNA (mRNA) therapeutic. In some embodiments, said ribonucleic acid (RNA) therapeutic is a non-coding RNA, a microRNA, a shRNA, a siRNA, or a combination thereof. In some embodiments, said extracellular vesicle comprises said RNA therapeutic in a fully intact or substantially intact form. In some embodiments, said RNA therapeutic is fully intact or substantially intact messenger RNA. In some embodiments, said RNA therapeutic comprises at least 5 copies of fully intact messenger RNA. In some embodiments, following said nanoelectroporation, on average, each extracellular vesicle released by said extracellular vesicle donor cell comprises at least one copy of said RNA therapeutic. In some embodiments, following said nanoelectroporation, on average, each extracellular vesicle released by said extracellular vesicle donor cell comprises at least one fully intact or substantially intact copy of said RNA therapeutic. In some embodiments, prior to said nanoelectroporation, said extracellular vesicle donor cell is a primary cell or a genetically-unmodified cell. In some embodiments, said extracellular vesicle donor cell is selected from the group consisting of: mouse embryonic fibroblasts (MEF), human embryonic fibroblasts (HEF), dendritic cells, mesenchymal stem cells, bone marrow-derived dendritic cells, bone marrow derived stromal cells, adipose stromal cells, endothelial cells, and immune cells. In some embodiments, said extracellular vesicle donor cell is not a neutrophil. In some embodiments, said extracellular vesicle is an exosome, a microvesicle, or an apoptotic body. In some embodiments, said extracellular vesicle is an exosome. In some embodiments, said polynucleotide is nanoelectroporated into said extracellular vesicle donor cell via a nanochannel located on a biochip. In some embodiments, said nanochannel comprises a diameter from 1 nanometer to 1000 nanometers. In some embodiments, said biochip comprises an array of nanochannels comprising a spacing between nanochannels from 1 micrometer to 100 micrometers. In some embodiments, said nanoelectroporation comprises an electric field. In some embodiments, said electric field has an electric field strength from 1 volt/mm to 1000 volt/mm In some embodiments, said electric field comprises a plurality of pulses with pulse durations from 0.1 milliseconds/pulse to 100 millisecond/pulse. In some embodiments, the tumor targeting domain is on a N-terminus of the tumor targeting polypeptide. In some embodiments, the tumor targeting domain is on a C-terminus of the tumor targeting polypeptide. In some embodiments, said muscular dystrophy is selected from the group consisting of: Duchenne muscular dystrophy, Becker muscular dystrophy, facioscapulohumeral muscular dystrophy, congenital muscular dystrophy, and myotonic dystrophy. In some embodiments, the muscular dystrophy is Duchenne muscular dystrophy.

Described herein, in some aspects, is a method for treating a retinal disease in a subject, said method comprising systemically administering at least one extracellular vesicle comprising a therapeutic to the subject, wherein said at least one extracellular vesicle comprising said therapeutic is obtained by: nanoelectroporating an extracellular vesicle donor cell with at least one polynucleotide, wherein said at least one polynucleotide encodes a targeting polypeptide that comprises: (i) an adapter polypeptide comprising a transmembrane domain and an extracellular domain; and (ii) a heterologous targeting domain that is covalently linked to said extracellular domain of said adapter polypeptide, and wherein said at least one polynucleotide encodes a ribonucleic acid (RNA) therapeutic; incubating said extracellular vesicle donor cell under conditions such that (i) said targeting polypeptide is expressed in said extracellular vesicle donor cell and (ii) said targeting polypeptide is incorporated into an extracellular vesicle released from said extracellular vesicle donor cell; and collecting said at least one extracellular vesicle released from said extracellular vesicle donor cell, wherein said at least one extracellular vesicle comprises said targeting polypeptide, wherein accumulation of the at least one extracellular vesicle at the tumor is higher compared to accumulation of an extracellular vesicle lacking the heterologous targeting domain. In some embodiments, said heterologous targeting domain is covalently linked to a N terminus of said extracellular domain of said adapter polypeptide. In some embodiments, said heterologous targeting domain is covalently linked to a C terminus of said extracellular domain of said adapter polypeptide. In some embodiments, said transmembrane domain of said adapter polypeptide is at least 70% identical to a transmembrane domain of a CD47 polypeptide or said extracellular domain of said adapter polypeptide is at least 70% identical to an extracellular domain of a CD47 polypeptide. In some embodiments, said adapter polypeptide is selected from the group consisting of CD63, CD81, CD82, CD47, CD315, heterotrimeric G protein, MHC class I, integrins, transferrin receptor (TFR2), LAMP1/2, heparan sulfate proteoglycans, EMMPRIN, ADAM10, GPI-anchored 5′nucleotidase, CD73, complement-binding protein CD55 and CD59, sonic hedgehog (SHH), TSPAN8, CD37, CD53, CD9, PECAM1, ERBB2, EPCAM, CD90, CD45, CD41, CD42a, Glycophorin A, CD14, MHC class II, CD3, Acetylcholinesterase/AChE-S, AChE-E, amyloid beta A4/APP, PTGFRN, and multidrug resistance-associated protein. In some embodiments, said adapter polypeptide is at least 70% identical to a polypeptide selected from the group consisting of: CD63, CD81, CD82, CD47, CD315, heterotrimeric G protein, MHC class I, integrins, transferrin receptor (TFR2), LAMP1/2, heparan sulfate proteoglycans, EMMPRIN, ADAM10, GPI-anchored 5′nucleotidase, CD73, complement-binding protein CD55 and CD59, sonic hedgehog (SHH), TSPAN8, CD37, CD53, CD9, PECAM1, ERBB2, EPCAM, CD90, CD45, CD41, CD42a, Glycophorin A, CD14, MHC class II, CD3, Acetylcholinesterase/AChE-S, AChE-E, amyloid beta A4/APP, PTGFRN, and multidrug resistance-associated protein. In some embodiments, said adapter polypeptide is at least 70% identical to CD47. In some embodiments, said heterologous targeting domain comprises a tumor targeting domain. In some embodiments, said tumor targeting domain is a CDX peptide. In some embodiments, said tumor targeting domain is a CREKA peptide. In some embodiments, said ribonucleic acid (RNA) therapeutic is incorporated into extracellular vesicles released from said extracellular vesicle donor cell and said method further comprises collecting said extracellular vesicles released from said extracellular vesicle donor cell. In some embodiments, said ribonucleic acid (RNA) therapeutic is a messenger RNA (mRNA) therapeutic. In some embodiments, said ribonucleic acid (RNA) therapeutic is a non-coding RNA, a microRNA, a shRNA, a siRNA, or a combination thereof. In some embodiments, said extracellular vesicle comprises said RNA therapeutic in a fully intact or substantially intact form. In some embodiments, said RNA therapeutic is fully intact or substantially intact messenger RNA. In some embodiments, said RNA therapeutic comprises at least 5 copies of fully intact messenger RNA. In some embodiments, following said nanoelectroporation, on average, each extracellular vesicle released by said extracellular vesicle donor cell comprises at least one copy of said RNA therapeutic. In some embodiments, following said nanoelectroporation, on average, each extracellular vesicle released by said extracellular vesicle donor cell comprises at least one fully intact or substantially intact copy of said RNA therapeutic. In some embodiments, prior to said nanoelectroporation, said extracellular vesicle donor cell is a primary cell or a genetically-unmodified cell. In some embodiments, said extracellular vesicle donor cell is selected from the group consisting of: mouse embryonic fibroblasts (MEF), human embryonic fibroblasts (HEF), dendritic cells, mesenchymal stem cells, bone marrow-derived dendritic cells, bone marrow derived stromal cells, adipose stromal cells, endothelial cells, and immune cells. In some embodiments, said extracellular vesicle donor cell is not a neutrophil. In some embodiments, said extracellular vesicle is an exosome, a microvesicle, or an apoptotic body.

In some embodiments, said extracellular vesicle is an exosome. In some embodiments, said polynucleotide is nanoelectroporated into said extracellular vesicle donor cell via a nanochannel located on a biochip. In some embodiments, said nanochannel comprises a diameter from 1 nanometer to 1000 nanometers. In some embodiments, said biochip comprises an array of nanochannels comprising a spacing between nanochannels from 1 micrometer to 100 micrometers. In some embodiments, said nanoelectroporation comprises an electric field. In some embodiments, said electric field has an electric field strength from 1 volt/mm to 1000 volt/mm In some embodiments, said electric field comprises a plurality of pulses with pulse durations from 0.1 milliseconds/pulse to 100 millisecond/pulse. In some embodiments, the tumor targeting domain is on a N-terminus of the tumor targeting polypeptide. In some embodiments, the tumor targeting domain is on a C-terminus of the tumor targeting polypeptide. In some embodiments, said retinal disease is retinitis pigmentosa. In some embodiments, said retinal disease is Leber's congenital amaurosis.

Described herein, in some aspects, is a method for treating a tumor in a subject comprising systemically administering at least one extracellular vesicle comprising a therapeutic polynucleotide to the subject, wherein the at least one extracellular vesicle comprising a therapeutic polynucleotide is obtained by: nanoelectroporating an extracellular vesicle donor cell with at least a first vector (e.g., plasmid) and at least a second vector (e.g., plasmid), wherein the first vector (e.g., plasmid) encodes a tumor or tissue targeting polypeptide comprising an extracellular vesicle surface protein covalently bound to a tumor or tissue targeting domain and the second vector encodes the therapeutic polynucleotide; expressing the first vector (e.g., plasmid) in the extracellular vesicle donor cell to obtain the tumor or tissue targeting polypeptide; transcribing the second vector (e.g., plasmid) in the extracellular vesicle donor cell to obtain the therapeutic polynucleotide; and collecting the at least one extracellular vesicle released from the extracellular vesicle donor cell; wherein accumulation of the at least one extracellular vesicle at the tumor or tissue of interest is higher compared to accumulation of an extracellular vesicle lacking the tumor or tissue targeting polypeptide. In some embodiments, the extracellular vesicle is an exosome. In some embodiments, accumulation of the at least one extracellular vesicle comprising the tumor or tissue targeting polypeptide at the tumor or tissue is at least 100-fold higher compared to accumulation of an extracellular vesicle lacking the tumor targeting polypeptide. In some embodiments, the extracellular vesicle donor cell is selected from the group consisting of: mouse embryonic fibroblasts (MEF), human embryonic fibroblasts (HEF), dendritic cells, mesenchymal stem cells, bone marrow-derived dendritic cells, bone marrow derived stromal cells, adipose stromal cells, endothelial cells, and immune cells. In some embodiments, the plurality of the first and second plasmids are nanoelectroporated into the extracellular vesicle donor cell via a nanochannel located on a biochip. In some embodiments, the nanochannel comprises a diameter from 1 nanometer to 1000 nanometers. In some embodiments, the biochip comprises an array of nanochannels comprising a spacing between nanochannels from 1 micrometer to 100 micrometers. In some embodiments, the nanoelectroporation comprises an electric field. In some embodiments, the electric field has an electric field strength from 1 volt/mm to 1000 volt/mm In some embodiments, the electric field comprises a plurality of pulses with pulse durations from 0.1 milliseconds/pulse to 100 millisecond/pulse. In some embodiments, the tumor or tissue targeting domain of the extracellular vesicles domain is on an N-terminus of the tumor or tissue targeting polypeptide. In some embodiments, the tumor targeting or tissue domain is on a C-terminus of the tumor targeting polypeptide. In some embodiments, the tumor or tissue targeting domain comprises a CDX peptide. In some embodiments, the tumor or tissue targeting domain comprises a CREKA peptide. In some embodiments, the extracellular vesicle surface protein of the extracellular vesicles comprises a peptide sequence at least 70% identical to a peptide sequence of a naturally occurring extracellular vesicle surface protein. In some embodiments, the naturally occurring extracellular vesicle surface protein is selected from the group consisting of: CD63, CD81, CD82, CD47, CD315, heterotrimeric G proteins, MHC class I, integrins, transferrin receptor (TFR2), LAMP1/2, heparan sulfate proteoglycans, EMMPRIN, ADAM10, GPI-anchored 5′nucleotidase, CD73, complement-binding proteins CD55 and CD59, and sonic hedgehog (SHH). In some embodiments, the naturally occurring extracellular vesicle surface protein is selected from the group consisting of: TSPAN8, CD37, CD53, CD9, PECAM1, ERBB2, EPCAM, CD90, CD45, CD41, CD42a, Glycophorin A, CD14, MHC class II, CD3, Acetylcholinesterase/AChE-S, AChE-E, amyloid beta A4/APP, PTGFRN, and multidrug resistance-associated protein. In some embodiments, the naturally occurring extracellular vesicle surface protein comprises CD47. In some embodiments, the at least one extracellular vesicle comprises at least 1 copy of the therapeutic polynucleotide. In some embodiments, the at least one extracellular vesicle comprises at least 2 copies, at least 5 copies, at least 10 copies, or at least 50 copies of the therapeutic polynucleotide. In some embodiments, the at least one extracellular vesicle comprises at least 100 copies of the therapeutic polynucleotide. In some embodiments, the at least one extracellular vesicle comprises at least 1000 copies of the therapeutic polynucleotide. In some embodiments, the therapeutic polynucleotide is selected from the group consisting of: mRNA, rRNA, SRP RNA, tRNA, tmRNA, snRNA, snoRNA, gRNA, aRNA, crRNA, lncRNA, miRNA, ncRNA, piRNA, siRNA, and shRNA. In some embodiments, the therapeutic polynucleotide comprises mRNA. In some embodiments, the mRNA comprises at least 100 RNA nucleotides. In some embodiments, the therapeutic polynucleotide comprises at least one modified nucleotide. In some embodiments, the therapeutic polynucleotide comprises a modified oligonucleotide. In some embodiments, the method described comprises treating a tumor with the extracellular vesicles. In some embodiments, the tumor is cancer. In some embodiments, the cancer is glioma.

Described herein, in some aspects, is a method for treating a muscular dystrophy in a subject comprising systemically administering at least one extracellular vesicle comprising a therapeutic polynucleotide to the subject, wherein the at least one extracellular vesicle comprising a therapeutic polynucleotide is obtained by: nanoelectroporating an extracellular vesicle donor cell with at least a first vector (e.g., plasmid) and at least a second vector (e.g., plasmid), wherein the first vector (e.g., plasmid) encodes a muscle cell targeting polypeptide comprising an extracellular vesicle surface protein covalently bound to a muscle cell targeting domain and the second vector encodes the therapeutic polynucleotide; expressing the first vector in the extracellular vesicle donor cell to obtain the muscle cell targeting polypeptide; transcribing the second vector in the extracellular vesicle donor cell to obtain the therapeutic polynucleotide; and collecting the at least one extracellular vesicle released from the extracellular vesicle donor cell. In some embodiments, the extracellular vesicle for treating the muscular dystrophy is an exosome. In some embodiments, the muscular dystrophy is selected from the group consisting of: Duchenne muscular dystrophy, Becker muscular dystrophy, facioscapulohumeral muscular dystrophy, congenital muscular dystrophy, and myotonic dystrophy. In some embodiments, the muscular dystrophy is Duchenne muscular dystrophy. In some embodiments, the therapeutic polynucleotide for treating muscular dystrophy comprises mRNA. In some embodiments, the therapeutic polynucleotide for treating muscular dystrophy comprises at least one modified nucleotide. In some embodiments, the therapeutic polynucleotide for treating muscular dystrophy comprises a modified oligonucleotide.

Described herein, in some aspects, is a method for treating a retinal disease in a subject comprising systemically administering at least one extracellular vesicle comprising a therapeutic polynucleotide to the subject, wherein the at least one extracellular vesicle comprising a therapeutic polynucleotide is obtained by: nanoelectroporating an extracellular vesicle donor cell with at least a first vector and at least a second vector, wherein the first vector encodes a retinal cell targeting polypeptide comprising an extracellular vesicle surface protein covalently bound to a retinal cell targeting domain and the second vector encodes the therapeutic polynucleotide; expressing the first vector in the extracellular vesicle donor cell to obtain the retinal cell targeting polypeptide; transcribing the second vector in the extracellular vesicle donor cell to obtain the therapeutic polynucleotide; and collecting the at least one extracellular vesicle released from the extracellular vesicle donor cell. In some embodiments, the extracellular vesicle for treating a retinal disease is an exosome. In some embodiments, the retinal disease is retinitis pigmentosa. In some embodiments, the retinal disease is Leber's congenital amaurosis.

Described herein, in some aspects, is a pharmaceutical composition comprising at least one extracellular vesicle, wherein the at least one extracellular vesicle comprises: at least one targeting polypeptide comprising an extracellular vesicle surface protein covalently bound to a targeting domain; and at least one therapeutic polynucleotide. In some embodiments, the pharmaceutical composition of the extracellular vesicle is an exosome. In some embodiments, the extracellular vesicle surface protein comprises an extracellular vesicle transmembrane domain In some embodiments, the extracellular vesicle transmembrane domain is at least 70% identical with a peptide sequence of CD47. In some embodiments, the extracellular vesicle of the pharmaceutical composition comprises at least two targeting domains. In some embodiments, the at least two targeting domains are different. In some embodiments, the therapeutic polynucleotide of the pharmaceutical composition is selected from the group consisting of: mRNA, rRNA, SRP RNA, tRNA, tmRNA, snRNA, snoRNA, gRNA, aRNA, crRNA, lncRNA, miRNA, ncRNA, piRNA, siRNA, and shRNA. In some embodiments, the therapeutic polynucleotide comprises mRNA. In some embodiments, the pharmaceutical composition is administered to a subject intrathecally, intraocularly, intravitreally, retinally, intravenously, intramuscularly, intraventricularly, intracerebrally, intracerebellarly, intracerebroventricularly, intraperenchymally, subcutaneously, or a combination thereof.

### BRIEF DESCRIPTION OF THE DRAWINGS

This patent application contains at least one drawing executed in color. Copies of this patent or patent application with color drawing(s) will be provided by the Office upon request and payment of the necessary fee.

The novel features of the disclosure are set forth with particularity in the appended claims. A better understanding of the features and advantages of the present disclosure will be obtained by reference to the following detailed description that sets forth illustrative embodiments.

FIG. 1A-1I illustrates Cellular Nanoporation (CNP) generating large quantities of extracellular vesicles (EVs) loaded with transcribed mRNAs. FIG. 1A. Schematic representation of CNP generated EVs for targeted nucleic acid delivery. Left: An exemplary CNP system consists of a nanochannel array, with each channel measuring about 500 nm in diameter (top inset). DNA vectors added in buffer enter attached cells through the nanochannels under transient electrical pulses. The attached cells subsequently released large quantities of exosomes containing transcribed mRNA that can be collected for tumor-targeted delivery via blood-brain barrier (BBB) and blood-brain tumor barrier (BBTB) (Right). FIG. 1B. EV number per cell produced by un-treated MEFs in PBS buffer (PBS), MEFs after treatment with Ascl1/Brn2/Myt1l (A/B/M) vectors transfected by lipofectamine 2000 (Lipo), bulk electroporation (BEP), and cellular nanoelectroporation (CNP), as well as CNP with only PBS buffer (CNP/PBS). FIG. 1C. Comparison of EV release by CNP method versus other traditional methods of stress-induced EV release including starvation, hypoxia and heat treatment. Starvation: MEF cells were cultured in DMEM without PBS; Hypoxia: MEF cells were cultured in a hypoxia chamber at 1% O₂ and 5% CO₂ at 37° C. humidified environment; Heat: MEF cells were cultured at 42° C. for 2 h and then transferred to 37° C. normal cell culture conditions. FIG. 1D. EV number per cell produced by mouse bone marrow-derived dendritic cells (BMDCs) in different treatment groups, including PBS, Lipo, BEP, CNP, and CNP/PBS groups. FIG. 1E. Exosome release from CNP-transfected MEFs peaks at around 8 h post-CNP. FIG. 1F. Dynamic light scattering (DLS) measurements of exosome concentration in MEFs by CNP at various voltages. Results showed that the exosome number did not increase when the voltage was increased from 200 to 220 V. **P<0.01, vs Voltage 0 V, #P<0.05, vs Voltage 150 V, Student t-test. FIG. 1G. Agarose gel analysis of EV-mRNAs collected from EVs after CNP. CNP/PBS: Total RNAs harvested from 107 MEFs after CNP with only PBS buffer; PTEN mRNA: 200 ng synthesized PTEN mRNA; CNP/PTEN; Total RNAs (˜1.0 μg) harvested from 107 MEFs after CNP with PTEN vector. FIG. 1H. qPCR of A, B, and M mRNA revealed that exosomes produced by CNP contained much larger quantities of transcribed mRNAs as compared with other methods. FIG. 1I. qPCR of EV A, B and M mRNA from CNP-transfected MEFs (in culture medium replaced every 4 h for 24 h) showed the largest transcript took longest to reach peak concentration. All data were from three independent experiments and were presented as mean±s.e.m. *P<0.05, **P<0.01, vs PBS, ##P<0.01, vs BEP, Student t-test (FIG. 1B, FIG. 1C, FIG. 1D, and FIG. 1H).

FIG. 2A-2I illustrates characterization of exosomes generated from CNP. FIG. 2A DLS measurement of vesicle size distribution produced by CNP. A peak around 70-110 nm was observed in the CNP group, indicating the massive production of exosomes by CNP. upper: PBS group, below: CNP group. FIG. 2B. DLS measurements of exosome number per cell in MEFs by gene gun at various pressures. Results showed that the EV number increased slightly with the increase of pressure used in gene gun. Data were from three independent experiments and were mean±s.e.m. *P<0.05, vs PBS, Student t-test. FIG. 2C. EV number per cell produced by mouse mesenchymal stem cells (MSCs) in different treatment groups, including PBS, Lipo, BEP, CNP, and CNP/PBS groups. FIG. 2D. EV number per cell produced by human embryonic kidney 293T (HEK293T) in different treatment groups, including PBS, Lipo, BEP, CNP, and CNP/PBS groups. FIG. 2E. EV number per cell produced by MEFs in CNP group at different temperatures of CNP operation. FIG. 2F. qPCR measurements of PTEN mRNA in EVs produced by various transfection methods with PTEN vector showed that EVs produced by CNP contained much larger quantities of transcribed PTEN mRNAs than other methods in MEFs. FIG. 2G. qPCR measurements of PTEN mRNA in EVs produced by various transfection methods with PTEN vector showed that EVs produced by CNP contained much larger quantities of transcribed PTEN mRNAs than other methods in BMDCs. FIG. 2H. qPCR measurements of miR-128 levels in EVs produced by various transfection methods with miR-128 vector showed that EVs produced by CNP contained much larger quantities of transcribed miR-128 than other methods in MEFs. FIG. 2I. Western blot of in vitro protein translation in total vesicles secreted from MEFs by different transfection methods, indicating that the total vesicles containing transcribed mRNA were able to translate into functional protein.

FIG. 3A-3F illustrates comparison of CNP with BEP on miRNA loading efficiency into exosomes. FIG. 3A. DLS measurement of vesicle size distribution produced by CNP in the exosome fraction collected by ultracentrifugation. FIG. 3B. DLS measurement of vesicle size distribution produced by CNP in the microvesicle (MV) fraction collected by ultracentrifugation. FIG. 3C. Representative TIRF images of TLN assay of miR-128 colocalized in exosomes (CD63-GFP) after CNP and BEP showed that CNP had a better miRNA-128-loading efficiency into exosomes compared to BEP. FIG. 3D. Colocalization percentage of miR-128 in exosomes after CNP and BEP. 100 images were used for statistical analysis. FIG. 3E. miR-128 fluorescence intensity within exosomes measured by TLN in CNP and BEP groups. 100 images were used for statistical analysis. FIG. 3F. DLS measurements of relative exosome numbers before and after BEP showed that BEP broke around 50% of exosomes. Data were from three independent experiments unless otherwise stated and were present as mean±s.e.m. *P<0.05, vs CNP, Student t-test (FIG. 3D, FIG. 3E, and FIG. 3F).

FIG. 4A-4H illustrates exosomes, other than microvesicles (MVs), containing functional transcribed-mRNAs after CNP. FIG. 4A. Detection of exosome markers (CD9, CD63, and Tsg101) and MV marker (Arf6) in the same amount (20 μg protein) of exosomes and MVs by Western blot. FIG. 4B. RNA amount in exosomes vs. in MVs from 108 CNP-transfected MEFs measured by Nanodrop, indicating that a majority of RNA is in exosomes as compared to MVs. FIG. 4C. Cryo-TEM images of exosomes from PBS group (PBS) and CNP group (CNP) showed no differences in the appearance of exosomes obtained from these two groups while exosomes contained more RNAs inside. FIG. 4D. qPCR of Ascl1 (A), Brn2 (B) and Myt1l (M) mRNA from exosomes and MVs showed that a majority of the transcribed mRNAs were in exosomes. FIG. 4E. In vitro protein translation from mRNA extracted from exosomes and MVs secreted by CNP-transfected MEFs. FIG. 4F. Schematic demonstration of the procedure for tethered lipoplex nanoparticle (TLN) assay. Nanoparticles containing specific molecular beacon (MB) were tethered onto a glass coverslip, and the exosomes were captured by nanoparticles. Hybridization of mRNA inside the exosomes with the MB inside the nanoparticles produced the fluorescence which was detected by total internal refractory microscopy (TIRF). FIG. 4G. Representative TIRF images of TLN assay in CNP and S—CNP groups showed that S-CNP optimized the loading of different mRNAs into individual exosomes. Medium gray (Green) dot: Ascl1 mRNA, dark gray (red) dots: Brn2 mRNA, light gray (purple) dots: Myt1l mRNA, dark gray (pink) arrow: exosomes with 1 mRNA, light gray (turquoise) arrow: exosomes with 2 mRNAs, medium gray (yellow) arrow: exosomes with 3 mRNAs. FIG. 4H. Percentage of exosomes with different RNAs in CNP and S—CNP groups. 100 images in each group were chosen for statistical analysis. **P<0.01, vs exosome, Student t-test (FIG. 4B and FIG. 4D).

FIG. 5A-D illustrates comparison of CNP with BEP on mRNA loading efficiency into exosomes. FIG. 5A. Representative images of TLN assay of Brn2 mRNA colocalized in exosomes (CD63-GFP) after CNP and BEP showed that CNP had a much higher mRNA loading efficiency into exosomes than BEP. FIG. 5B. Colocalization percentage of Brn2 mRNA in exosomes after CNP and BEP. 100 images were used for statistical analysis. FIG. 5C. Brn2 mRNA fluorescence intensity within EVs as measured by TLN in CNP and BEP groups. 100 images were used for statistical analysis. FIG. 5D. qPCR of miR-128 and Brn2 mRNA expression (CT value) of exosomes secreted from 10⁷ CNP-transfected MEFs (CNP), free RNA from 10⁷ CNP-transfected MEFs mixed with exosomes from 10⁷ CNP/PBS transfected MEFs (Mixture), exosomes from Mixture after bulk electroporation-based RNA insertion (BEP w/o RNase), and RNase treated exosomes from Mixture after BEP to remove RNA molecules attached on exosome outer surface (BEP w RNase). All data were from three independent experiments and were present as mean±s.e.m. **P<0.01, vs CNP, ##P<0.01, vs BEP w/o RNase, Student t-test (FIG. 5B, FIG. 5C, and FIG. 5D).

FIG. 6A-6O illustrates CNP-induced exosome secretion was associated with Ca²⁺ ion influx after CNP. FIG. 6A. Epi-fluorescence images showing increased intracellular vesicle formation in MEFs with CNP/PBS stimulation as measured by red fluorescence spots from PKH26 dye. FIG. 6B. CNP/PBS-porated MEFs (CNP) resulted in increased formation of multivesicular body (MVB) containing CD63-GFP as compared BEP. Insets: 3D intensity profiles in which peaks represented bright spots in images indicating active MVB formation. FIG. 6C. Transmission electron microscopy (TEM) images of MEFs with or without CNP/PBS stimulation contained different quantities of MVBs and intraluminal vesicles (ILVs). FIG. 6D and FIG. 6E. Quantification of MVBs (FIG. 6D) and ILVs (FIG. 6E) in MEFs with or without CNP/PBS stimulation. n=20 TEM images for each group. FIG. 6F. Western blot showing the proteins implicated in exosome biogenesis were increased after CNP. FIG. 6G. Longitudinal fluorescence intensity measurement of propidium iodide (PI) diffusion across membrane pores in BEP- and CNP-porated MEFs with PBS buffer. Rapid increase in PI intensity at the attached surface of the cell (top insert) indicated formation of an array of large pores, whereas a much slower PI increase at the contralateral cell surface (bottom insert) indicated formation of smaller pores. BEP-porated MEFs showed an intermediate increase in PI intensity. FIG. 6H. Fluorescence images of cells after CNP indicated the membrane pores formed during CNP close between 1 to 2 min after transfection. PI was applied to the cells at indicated time points after CNP. FIG. 6I. Fluorescence intensity measurement of cells further confirmed membrane pores close within 2 min following CNP. n=20 cells for each group. FIG. 6J. Exosome number per cell produced by MEFs at various calcium ion concentrations after CNP. FIG. 6K. Intracellular calcium ion concentration after CNP at various calcium ion concentrations in buffer. FIG. 6L. Correlation of exosome release with intracellular calcium ion concentration after CNP. FIG. 6M. Exosome number per cell produced by MEF at various calcium ion concentrations after CNP with the presence of calcium chelator, EGTA. FIG. 6N. Calcium ion concentration inside the cells after CNP at various calcium ion concentrations in buffer with the presence of EGTA. FIG. 6O. Correlation of exosome release with intracellular calcium ion concentration after CNP with the presence of EGTA.

FIG. 7A-7K Thermal effects of CNP increased exosome release through HSP-P53-TASP6 signaling pathway. FIG. 7A. Schematic demonstration of simulated temperature rise in a single nanochannel FIG. 7B. Selected 5 different locations in/near nanochannel. FIG. 7C. Simulated temperature changes at 5 chosen locations. A 200 V and 10 ms pulse created a localized “hot spot” in the nanochannel outlet and a peak temperature up to 60° C. from ambient temperature. Once the pulse ended, the ‘hot spot’ would vanish rapidly. FIG. 7D. Top-down images of MEFs attaching to CNP device surface. Before CNP (0 s), dots indicated nanochannel locations and room temperature. CNP electric pulse (CNP) sharply increased temperature at nanochannel/cell surface interface. FIG. 7E. Cross-section view of nanochannels showed temperature changes within the nanochannels before (0 s), during and post (1 s) a CNP pulse. FIG. 7F. Temperature measured at the cell-nanochannel interface transiently (<1 s) increases to ˜60° C. FIG. 7G. Western blot of HSP90 and HSP70 from un-treated (PBS) and CNP/PBS-stimulated (CNP) MEFs. FIG. 7H. DLS measurements of exosome concentrations of 108 CNP-stimulated MEFs with or without HSP inhibitors show that HSP70 and HSP90 were critical to the production of exosomes. NVP-HSP990: HSP90 inhibitor; VER155008: HSP70 inhibitor. **P<0.01, vs CNP, ##P<0.01 vs single inhibitor group, Student t-test. FIG. 7I. Western blot results showed CNP increased the P53 and TSAP6 protein expression in P53 WT MEFs while it did not affect the P53 or TSAP6 protein expression in p53−/− MEFs. FIG. 7J. DLS measurements of exosome concentrations showed the knockdown of P53 could partially block the exosome release after CNP. ##P<0.01 vs CNP-P53+/+ group, Student t-test. FIG. 7K. Schematic of a proposed mechanism for how CNP triggered exosomes release in CNP-transfected cells. Data were from three independent experiments and were present as mean±s.e.m.

FIG. 8A-8L illustrates in vitro study of CNP generated exosomes for gene therapy and immunogenicity evaluation in mice. FIG. 8A. Schematic representation of glioblastoma (GBM) targeting peptide cloned into N-terminal of CD47 transmembrane protein. FIG. 8B. Western blots of exosome pulldown assay showed that FLAG beads were able to pull down the N-terminal cloned FLAG-CD47, indicating that the N-terminal of CD47 was outside of the exosomes. FIG. 8C. Increased uptake of CNP-generated exosomes coated with a brain tumor targeting peptide linked to CD47 by gliomas (GL261) cells. Exosome: uncoated exosomes. Exo-T: exosomes generated from CNP stimulated BMDCs transfected with CREKA-CD47 vector. FIG. 8D. Fluorescence intensity of PKH26-labeled Exo-T taken up by GL261 as assessed by flow cytometry indicated that the Exo-T had the better uptake in GL261 cells. FIG. 8E. Representative confocal microscopy images of PTEN staining in GL261 cells 24 h after PBS, exosome or Exo-T treatments. FIG. 8F. Flow cytometry measurement of fluorescence intensity of PTEN staining 24 hours after incubation of GL261 with exosomes showed the Exo-T group had stronger PTEN protein expression. FIG. 8G. Representative immunostaining images of co-localization of PKH26-labeled Exo-T vesicles (red) with different endocytosis markers (green). Results indicated the majority of Exo-Ts were co-localized with A488-Tf, indicating Exo-Ts were mainly taken up through clathrin-dependent endocytosis. A488-Tf: Clathrin-dependent endocytosis marker; A488-CT-B: Caveolae-dependent endocytosis marker; and FITC-dextran: Macropinocytosis marker. FIG. 8H. Fluorescence intensity of PKH26-labeled Exo-T uptake by GL261 under different inhibition conditions by flow cytometry further showed that Exo-Ts were primarily taken up through clathrin-dependent endocytosis. Sucrose: Clathrin-dependent endocytosis inhibitor; Filipin: Caveolae-dependent endocytosis inhibitor, and Wortinin: Macropinocytosis inhibitor. FIG. 8I. GL261 cell viability treated by empty lipofectamine (E-Lipo), exosome and Exo-T indicated good biocompatibility of the Exo-T. FIG. 8J. GL261 cell viability treated by lipofectamine, exosome and Exo-T containing PTEN mRNA. FIG. 8K. Circulatory half-life of systemically administered PKH26-labeled exosomes in mice. Overexpression of CD47 protein greatly extended the circulatory half-life of exosomes, which was not affected by the insertion of CREKA peptide. Exo-C: exosomes from CNP/CD47 vector-transfected BMDCs. Exo-T: exosomes from CNP/CREKA-CD47 vector-transfected BMDCs. Inset: Confirmation of CD47 protein expression in exosomes from BMDCs transfected with CREKA-CD47 vector. FIG. 8L. AST, ALT, creatinine, BUN, IL6 and TNF. levels measured by ELISA with administration of different doses of CREKA-CD47 targeted exosomes (Exo-Ts). *P<0.05, **P<0.01, vs PBS, ##P<0.01 vs exosome. Student t-test (FIG. 8D, FIG. 8F, and FIG. 8H)

FIG. 9 illustrates an exemplary gating strategy for flow cytometry analysis of exosome targeting.

FIG. 10A-10I illustrates in vitro study of CNP generated exosomes for gene therapy in U87 cells. FIG. 10A. Increased uptake of CNP-generated exosomes coated with a brain tumor targeting peptide (CDX) linked to CD47 by glioma (U87) cells. Exosome: uncoated exosomes. Exo-T: exosomes generated from CNP stimulated MEFs transfected with CDX-CD47 vector. FIG. 10B. Fluorescence intensity of PKH26-labeled Exo-T taken up by U87 by flow cytometry further confirmed Exo-T had the better uptake in U87 cells. FIG. 10C. Representative confocal microscopy images of PTEN staining in U87 cells 24 h after PBS, exosome or Exo-T treatments. FIG. 10D. Fluorescence intensity of PTEN staining 24 h after incubation of U87 with exosomes by flow cytometry showed the Exo-T group had the stronger PTEN protein expression. FIG. 10E. Representative immunostaining images of co-localization of PKH26-labeled Exo-T vesicles (red) with different endocytosis markers (green). Results indicated the majority of Exo-Ts were co-localized with A488-Tf, indicating Exo-Ts were mainly taken up through clathrin-dependent endocytosis. A488-Tf: Clathrin-dependent endocytosis marker; A488-CT-B: Caveolae-dependent endocytosis marker; and FITC-dextran: Macropinocytosis marker. FIG. 10F. Fluorescence intensity of PKH26-labeled Exo-T taken up by U87 under different inhibition conditions by flow cytometry further confirmed Exo-Ts were mainly taken up through clathrin-dependent endocytosis. Sucrose: Clathrin-dependent endocytosis inhibitor; Filipin: Caveolae-dependent endocytosis inhibitor, and Wortinin: Macropinocytosis inhibitor. FIG. 10G. U87 cell viability treated by empty lipofectamine (E-Lipo), exosome and Exo-T indicated good biocompatibility of the Exo-T. FIG. 10H. U87 cell viability treated by lipofectamine, exosome and Exo-T containing PTEN mRNA. FIG. 10I. AST, ALT, creatinine, BUN, IL6 and TNF. levels measured by ELISA at various time points in mice with different types of exosomes. Results showed that Exo-T had no obvious in vivo toxicity and immunogenicity in mice.

FIG. 11A-11M illustrates in vivo therapeutic efficacy of CNP-generated exosomes in a U87 orthotopic glioma model. FIG. 11A. In vivo imaging showing preferential accumulation of PKH-26 labeled Exo-T within orthotopically implanted U87 tumors in nude mice. The targeted delivery of Exo-T into brain tumors was also confirmed by intravital fluorescence microscopy (FIG. 11B) which showed significantly increased accumulation of Exo-T within the tumor stroma as compared with uncoated exosomes (exosome) or TurboFect nanoparticles (Turbo). FIG. 11C. Quantification of exosome intensity in the tumor site at various time points. Ten images per animal with 3 mice per group. FIG. 11D and FIG. 11E. Tissue distribution analyses showed Exo-T exhibited increased brain targeting with low hepatic and splenic accumulation. FIG. 11F and FIG. 11G. Tumor growth inhibition by PBS, PTEN mRNA containing exosomes (exosome), Exo-T, empty Exo-T (E-Exo-T), or TurboFect nanoparticles (Turbo) treatment via tail vein injection. n=3 mice per group. FIG. 11H. PTEN mRNA Exo-T extended the survival of mice with U87 glioma (p<0.001, Log-rank test after Bonferroni correction). n=8 mice per group. FIG. 11I and FIG. 11J. Western blots (FIG. 11I) and qPCR (FIG. 11J) of PTEN protein and mRNA levels respectively in GBM tumors, indicated the restoration of both PTEN protein and mRNA expression in PTEN-null U87 GBM tumor. n=3 mice per group. FIG. 11K. PTEN, Ki67 and H&E staining of residue GBM tumor tissue with different treatments showed that Exo-T restored the PTEN expression and inhibited the cell proliferation in tumor tissue. FIG. 11L. Ki67 intensity measurement of IHC images by ImageJ software. FIG. 11M. PTEN intensity measurement of IHC images by ImageJ software. Data were from three independent experiments unless otherwise stated and were present as mean±s.e.m. *P<0.05, **P<0.01, vs PBS, ##P<0.01 vs exosome group, Student t-test (FIG. 11C, FIG. 11E, FIG. 11G, FIG. 11H, FIG. 11J, FIG. 11L, and FIG. 11M).

FIG. 12A-12B illustrates in vivo biodistribution of Exo-Ts within the tumor interstitium. FIG. 12A.

Representative intravital fluorescence images in mouse GBM tumor stroma at various time points post administration of different nanocarriers labeled with PKH26 showed that Exo-T had a better GBM tumor accumulation compared to other treatments. n=3 mice for each group. FIG. 12B. Segmentation of the exosomes conjugated with PKH26 from the whole image.

FIG. 13A-13M illustrates immunohistochemistry staining of different tissues in a U87 orthotopic glioma model. FIG. 13A. PTEN, Ki67 and H&E staining of normal brain tissue with different treatments showed no direct effect on normal brain tissue. FIG. 13B-F. PTEN and H&E staining of heart, liver, spleen, lung and kidney tissue with different treatments showed that Exo-T exhibited no effect on the tissues examined. Magnification: ×400. FIG. 13G-M. Ki67 and PTEN intensity measurement of IHC images by ImageJ software.

FIG. 14A-14N illustrates in vivo therapeutic efficacy of CNP-generated exosomes in a GL261 orthotopic glioma model. FIG. 14A. In vivo imaging showing preferential accumulation of PKH-26 labeled Exo-T within orthotopically implanted GL261 tumors in C57BL/6 mice. The targeted delivery of Exo-T into brain tumors was also confirmed by intravital fluorescence microscopy (FIG. 14B) which showed significantly increased accumulation of Exo-T within the tumor stroma as compared with uncoated exosomes (exosome) or PEG-liposome nanoparticles (Liposome). FIG. 14C. Quantification of exosome intensity in the tumor site at various time points. FIG. 14D. Distribution of PBS (Top row) and Exo-T (Bottom row) conjugated with PHK26 within normal tissue area and tumor area, scale bar: 500 μm. FIG. 14E. and FIG. 14F. Tissue distribution analyses showed Exo-T exhibited increased brain targeting with low hepatic and splenic accumulation. FIG. 14G and FIG. 14H. Tumor growth inhibition by PBS, PTEN mRNA containing exosomes (exosome), Exo-T, empty Exo-T (E-Exo-T), or PEG-liposome nanoparticles (Liposome) treatment via tail vein injection. n=3 mice per group. FIG. 14I. PTEN mRNA Exo-T extended the survival of mice with GL261 glioma (p<0.001, Log-rank test after Bonferroni correction). n=8 mice per group. FIG. 14J and FIG. 14K. Western blots (FIG. 14J) and qPCR (FIG. 14K) of PTEN protein and mRNA levels respectively in GBM tumors, showed the restoring of both PTEN protein and mRNA expression in PTEN-null GL261 GBM tumor. n=3 mice per group. FIG. 14L. PTEN, Ki67 and H&E staining of residue GBM tumor tissue with different treatments showed that Exo-T restored the PTEN expression and inhibited the cell proliferation in tumor tissue. FIG. 14M. Ki67 intensity measurement of IHC images by ImageJ software. FIG. 14N. PTEN intensity measurement of IHC images by ImageJ software. Data were from three independent experiments unless otherwise stated and were present as mean±s.e.m. *P<0.05, **P<0.01, vs PBS, ##P<0.01 vs exosome group, Student t-test (FIG. 14C, FIG. 14F, FIG. 14H, FIG. 14I, FIG. 14K, FIG. 14M, and FIG. 14N).

FIG. 15A-15M illustrates immunohistochemistry staining of different tissues in a GL261 orthotopic glioma model. FIG. 15A. PTEN, Ki67 and H&E staining of normal brain tissue with different treatments showed no direct effect on normal brain tissue. FIG. 15B-F. PTEN and H&E staining of heart, liver, spleen, lung and kidney tissue with different treatments showed that Exo-T exhibited no effect on the tissues examined. Magnification: ×400. Spleen: 100×. FIG. 15G-M. Ki67 and PTEN intensity measurement of IHC images by ImageJ software.

### DETAILED DESCRIPTION

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the disclosure and that methods and structures within the scope of these claims and their equivalents be covered thereby.

Use of absolute or sequential terms, for example, “will,” “will not,” “shall,” “shall not,” “must,” “must not,” “first,” “initially,” “next,” “subsequently,” “before,” “after,” “lastly,” and “finally,” are not meant to limit scope of the present embodiments disclosed herein but as exemplary.

As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising.”

As used herein, the phrases “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together.

As used herein, “or” may refer to “and”, “or,” or “and/or” and may be used both exclusively and inclusively. For example, the term “A or B” may refer to “A or B”, “A but not B”, “B but not A”, and “A and B”. In some cases, context may dictate a particular meaning.

Any systems, methods, software, and platforms described herein are modular and not limited to sequential steps. Accordingly, terms such as “first” and “second” do not necessarily imply priority, order of importance, or order of acts.

As used herein, the term “about” when referring to a number or a numerical range means that the number or numerical range referred to is an approximation within experimental variability (or within statistical experimental error), and the number or numerical range may vary from, for example, from 1% to 15% of the stated number or numerical range. Unless otherwise indicated by context, the term “about” refers to ±10% of a stated number or value.

The term “approximately” means within an acceptable error range for the particular value as determined by one of ordinary skill in the art, which will depend in part on how the value is measured or determined, e.g., the limitations of the measurement system. For example, “approximately” can mean within 1 or more than 1 standard deviation, per the practice in the given value. Where particular values are described in the application and claims, unless otherwise stated the term “approximately” should be assumed to mean an acceptable error range for the particular value.

The terms “increased”, “increasing”, or “increase” are used herein to generally mean an increase by a statically significant amount. In some cases, the terms “increased,” or “increase,” mean an increase of at least 10% as compared to a reference level, for example an increase of at least about 10%, at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, standard, or control. Other examples of “increase” include an increase of at least 2-fold, at least 5-fold, at least 10-fold, at least 20-fold, at least 50-fold, at least 100-fold, at least 1000-fold or more as compared to a reference level.

The terms, “decreased”, “decreasing”, or “decrease” are used herein generally to mean a decrease by a statistically significant amount. In some cases, “decreased” or “decrease” means a reduction by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (e.g., absent level or non-detectable level as compared to a reference level), or any decrease between 10-100% as compared to a reference level. In the context of a marker or symptom, by these terms is meant a statistically significant decrease in such level. The decrease can be, for example, at least 10%, at least 20%, at least 30%, at least 40% or more, and is preferably down to a level accepted as within the range of normal for an individual without a given disease.

The terms “patient” or “subject” are used interchangeably herein, and encompass mammals Non-limiting examples of mammal include, any member of the mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like.

As used herein, a “cell” generally refers to a biological cell. A cell is the basic structural, functional and/or biological unit of a living organism. A cell can originate from any organism having one or more cells. Some non-limiting examples include: a prokaryotic cell, eukaryotic cell, a bacterial cell, an archaeal cell, a cell of a single-cell eukaryotic organism, a protozoa cell, a cell from a plant, a fungal cell (e.g., a yeast cell, a cell from a mushroom), an animal cell, a cell from an invertebrate animal (e.g. fruit fly, cnidarian, echinoderm, nematode, etc.), a cell from a vertebrate animal (e.g., fish, amphibian, reptile, bird, mammal), or a cell from a mammal (e.g., a pig, a cow, a goat, a sheep, a rodent, a rat, a mouse, a non-human primate, a human, etc.). Sometimes a cell is not originating from a natural organism (e.g. a cell is a synthetically made, sometimes termed an artificial cell). In some cases, the cell is a primary cell. In some cases, the cell is derived from a cell line.

The term “nucleotide,” as used herein, generally refers to a base-sugar-phosphate combination. A nucleotide comprises a synthetic nucleotide. A nucleotide comprises a synthetic nucleotide analog. Nucleotides is monomeric units of a nucleic acid sequence (e.g. deoxyribonucleic acid (DNA) and ribonucleic acid (RNA)). The term nucleotide can include ribonucleoside triphosphates adenosine triphosphate (ATP), uridine triphosphate (UTP), cytosine triphosphate (CTP), guanosine triphosphate (GTP) and deoxyribonucleoside triphosphates such as dATP, dCTP, dITP, dUTP, dGTP, dTTP, or derivatives thereof. Such derivatives can include, for example, [αS]dATP, 7-deaza-dGTP and 7-deaza-dATP, and nucleotide derivatives that confer nuclease resistance on the nucleic acid molecule containing them. The term nucleotide as used herein can refer to dideoxyribonucleoside triphosphates (ddNTPs) and their derivatives. Illustrative examples of dideoxyribonucleoside triphosphates can include, but are not limited to, ddATP, ddCTP, ddGTP, ddITP, and ddTTP.

The terms “polynucleotide,” “oligonucleotide,” and “nucleic acid” are used interchangeably to refer to a polymeric form of nucleotides of any length, either deoxyribonucleotides or ribonucleotides, or analogs thereof, either in single-, double-, or multi-stranded form. In some cases, a polynucleotide is exogenous (e.g. a heterologous polynucleotide). In some cases, a polynucleotide is endogenous to a cell. In some cases, a polynucleotide can exist in a cell-free environment. In some cases, a polynucleotide is a gene or fragment thereof. In some cases, a polynucleotide is DNA. In some cases, a polynucleotide is RNA. A polynucleotide can have any three dimensional structure, and can perform any function, known or unknown. In some cases, a polynucleotide comprises one or more analogs (e.g. altered backbone, sugar, or nucleobase). If present, modifications to the nucleotide structure may be imparted before or after assembly of the polymer. Some non-limiting examples of analogs include: 5-bromouracil, peptide nucleic acid, xeno nucleic acid, morpholinos, locked nucleic acids, glycol nucleic acids, threose nucleic acids, dideoxynucleotides, cordycepin, 7-deaza-GTP, fluorophores (e.g. rhodamine or fluorescein linked to the sugar), thiol containing nucleotides, biotin linked nucleotides, fluorescent base analogs, CpG islands, methyl-7-guanosine, methylated nucleotides, inosine, thiouridine, pseudourdine, dihydrouridine, queuosine, and wyosine. Non-limiting examples of polynucleotides include coding or non-coding regions of a gene or gene fragment, loci (locus) defined from linkage analysis, exons, introns, messenger RNA (mRNA), transfer RNA (tRNA), ribosomal RNA (rRNA), short interfering RNA (siRNA), short-hairpin RNA (shRNA), micro-RNA (miRNA), non-coding RNA, ribozymes, cDNA, recombinant polynucleotides, branched polynucleotides, plasmids, vectors, isolated DNA of any sequence, isolated RNA of any sequence, cell-free polynucleotides including cell-free DNA (cfDNA) and cell-free RNA (cfRNA), nucleic acid probes, and primers. In some cases, the sequence of nucleotides is interrupted by non-nucleotide components.

“Fully intact” and “substantially intact” refer to a nucleic acid described herein having a nucleic acid sequence that can be transcribed and/or translated into a therapeutic polypeptide described herein. Fully intact nucleic acid refers to full-length nucleic acid sequence, which is not partially degraded or fragmented. For example, a fully intact nucleic acid can be a messenger RNA that can be translated into a full-length protein such as any one of the therapeutic polypeptides described herein. In general, a fully intact or substantially intact messenger RNA is capable of being translated into a polypeptide. Generally, messenger RNA comprises a 5′ cap which may assist with binding to a ribosome and a poly (A) tail, which may be useful for translation. The term “substantially intact” refers to a nucleic acid sequence that can be partially degraded or fragmented but still can be transcribed and/or translated into any one of the therapeutic polypeptides described herein. For example, a substantially intact nucleic acid can be a partially degraded or fragmented messenger RNA that can be translated into any one of the therapeutic polypeptides described herein.

As used herein, the terms “polypeptide”, “peptide”, and “protein” can be used interchangeably herein in reference to a polymer of amino acid residues. A polypeptide can refer to a full-length polypeptide as translated from a coding open reading frame, or as processed to its mature form. A polypeptide can refer to a degradation fragment or a processing fragment of a protein that nonetheless uniquely or identifiably maps to a particular protein. A polypeptide can be a single linear polymer chain of amino acids bonded together by peptide bonds between the carboxyl and amino groups of adjacent amino acid residues. A polypeptide can be modified, for example, by the addition of carbohydrate, phosphorylation, etc. A polypeptide can be a heterologous polypeptide.

As used herein, the terms “fragment” or equivalent terms can refer to a locus of a protein that has less than the full length of the protein and optionally maintains the function of the protein. “Percent identity” and “% identity” refers to the extent to which two sequences (nucleotide or amino acid) have the same residue at the same positions in an alignment. For example, “an amino acid sequence is X % identical to SEQ ID NO: Y” refers to % identity of the amino acid sequence to SEQ ID NO:Y and is elaborated as X % of residues in the amino acid sequence are identical to the residues of sequence disclosed in SEQ ID NO: Y. Generally, computer programs are employed for such calculations. Exemplary programs that compare and align pairs of sequences, include ALIGN, FASTA, gapped BLAST, BLASTP, BLASTN, or GCG.

As used herein, the term “in vivo” is used to describe an event that takes place in a subject's body.

As used herein, the term “ex vivo” is used to describe an event that takes place outside of a subject's body. An “ex vivo” assay cannot be performed directly on a subject. Rather, it is performed upon a sample separate from a subject, such as a biological sample obtained from the subject. Ex vivo is used to describe an event occurring in an intact cell or other type of biological sample outside a subject's body.

As used herein, the term “in vitro” is used to describe an event that takes place contained in a container for holding a laboratory reagent such that it is separated from the living biological source organism from which the material is obtained. In vitro assays can encompass cell-based assays in which live or dead cells or other biological materials are employed. In vitro assays can also encompass a cell-free assay in which no intact cells are employed.

“Treating” or “treatment” can refer to both therapeutic treatment and prophylactic or preventative measures, wherein the object is to prevent or slow down (lessen) a targeted pathologic condition or disorder. Those in need of treatment include those already with the disorder, as well as those prone to have the disorder, or those in whom the disorder is to be prevented. A therapeutic benefit can refer to eradication or amelioration of symptoms or of an underlying disorder being treated. Also, a therapeutic benefit is achieved with the eradication or amelioration of one or more of the physiological symptoms associated with the underlying disorder such that an improvement is observed in the subject, notwithstanding that the subject can still be afflicted with the underlying disorder. A prophylactic effect can include delaying, preventing, or eliminating the appearance of a disease or condition, delaying or eliminating the onset of symptoms of a disease or condition, slowing, halting, or reversing the progression of a disease or condition, or any combination thereof. A prophylactic benefit, a subject at risk of developing a particular disease, or to a subject reporting one or more of the physiological symptoms of a disease can undergo treatment, even though a diagnosis of this disease cannot have been made.

The term “effective amount” and “therapeutically effective amount,” as used interchangeably herein, generally refer to the quantity of a pharmaceutical composition, for example a pharmaceutical composition comprising a composition described herein, that is sufficient to result in a desired activity upon administration to a subject in need thereof. Within the context of the present disclosure, the term “therapeutically effective” refers to that quantity of a pharmaceutical composition that can be sufficient to delay the manifestation, arrest the progression, relieve or alleviate at least one symptom of a disorder treated by the methods of the present disclosure.

The term “pharmaceutically acceptable carrier,” “pharmaceutically acceptable excipient,” “physiologically acceptable carrier,” or “physiologically acceptable excipient” refers to a pharmaceutically-acceptable material, composition, or vehicle, such as a liquid or solid filler, diluent, excipient, solvent, or encapsulating material. A component is “pharmaceutically acceptable” in the sense of being compatible with the other ingredients of a pharmaceutical formulation. It can also be suitable for use in contact with the tissue or organ of humans and non-human mammals without excessive toxicity, irritation, allergic response, immunogenicity, or other problems or complications, commensurate with a reasonable benefit/risk ratio.

The term “pharmaceutical composition” refers to the systems or a mixture of the systems or compositions comprising each component of the systems disclosed herein with other chemical components, such as diluents or carriers. The pharmaceutical composition can facilitate administration of the systems or components of the systems to the subject. Multiple techniques of administering a compound exist in the art including, but not limited to, oral, injection, aerosol, parenteral, and topical administration.

The terms “transfection” or “transfected” generally refers to introduction of a nucleic acid construct into a cell by non-viral or viral-based methods. In some cases, the nucleic acid molecules are gene sequences encoding complete proteins or functional portions thereof. In some cases, the nucleic acid molecules are non-coding sequences. In some cases, the transfection methods are utilized for introducing nucleic acid molecules into a cell for generating a transgenic animal. Such techniques can include pronuclear microinjection, retrovirus mediated gene transfer into germ lines, gene targeting into embryonic stem cells, electroporation of embryos, sperm mediated gene transfer, and in vitro transformation of somatic cells, such as cumulus or mammary cells, or adult, fetal, or embryonic stem cells, followed by nuclear transplantation.

“Nanoelectroporation” or “nanochannel electroporation” refers to transfecting a cell with at least one heterologous polynucleotide such as a vector by loading the at least one heterologous polynucleotide into a nanochannel and accelerating the at least on heterologous polynucleotide into the cell with by generating an electric field. The cell to be transfected is situated at an opening of the nanochannel, where the electric field of the nanoelectroporation creates pores in the cell membrane to allow the at least one heterologous polynucleotide to be introduced into the cell.

A “plasmid,” as used herein, generally refers to a non-viral expression vector, e.g., a nucleic acid molecule that encodes for genes and/or regulatory elements necessary for the expression of genes. The term “vector,” as used herein, generally refers to a nucleic acid molecule capable transferring or transporting a payload nucleic acid molecule. The payload nucleic acid molecule can be generally linked to, e.g., inserted into, the vector nucleic acid molecule. A vector can include sequences that direct autonomous replication in a cell, or can include sequences sufficient to allow integration into host cell gene (e.g., host cell DNA). Examples of a vector can include, but are not limited to, plasmids (e.g., DNA plasmids or RNA plasmids), transposons, cosmids, bacterial artificial chromosomes, and viral vectors. A “viral vector,” as used herein, generally refers to a viral-derived nucleic acid that is capable of transporting another nucleic acid into a cell. A viral vector is capable of directing expression of a protein or proteins encoded by one or more genes carried by the vector when it is present in the appropriate environment. Examples for viral vectors include, but are not limited to Gamma-retroviral, Alpha-retroviral, Foamy viral, lentiviral, adenoviral, or adeno-associated viral vectors. A vector of any of the aspects of the present disclosure can comprise exogenous, endogenous, or heterologous control sequences such as promoters and/or enhancers.

### Overview

The present disclosure relates to the design and production of one or more extracellular vesicles (e.g., exosomes) that express at least one targeting polypeptide and/or carry a therapeutic cargo (e.g., mRNA). The targeting polypeptide can, in some instances, increase the targeting and accumulation of the extracellular vesicles to a targeted cell such as a diseased cell, a cancer cell, a tumor cell, a non-cancer lesion cell, a cell in damaged tissue, or a cell in healthy tissue. In some cases, the targeting polypeptide is a tumor targeting polypeptide. The targeting polypeptide can comprise an adapter polypeptide comprising a transmembrane domain and an extracellular domain. The targeting polypeptide can also comprise a heterologous targeting domain that is linked to the extracellular domain of the adapter polypeptide. The extracellular vesicles can be designed to carry a payload such as a therapeutic to be delivered to the targeted cell. In some cases, the therapeutic delivered by the extracellular vesicles can include a therapeutic compound (e.g., a therapeutic polynucleotide, therapeutic DNA, therapeutic RNA, therapeutic mRNA, therapeutic miRNA, therapeutic tRNA, therapeutic rRNA, therapeutic siRNA, therapeutic shRNA, therapeutic SRP RNA, therapeutic tmRNA, therapeutic gRNA, or therapeutic crRNA). In some cases, the therapeutic delivered by the extracellular vesicle can include a therapeutic non-coding polynucleotide (e.g., non-coding RNA, lncRNA, piRNA, snoRNA, snRNs, exRNA, or scaRNA), therapeutic polypeptide, therapeutic compound, or cancer drug. In some cases, the extracellular vesicles may carry a non-therapeutic compound (e.g., non-therapeutic polynucleotide).

This disclosure provides methods of producing large number of exosomes containing high quantity of mRNA transcripts, even from cells with otherwise low basal secretion of exosomes. One approach provided herein involves nanoelectroporating at least one heterologous polynucleotide such as a vector (e.g., plasmid) into an extracellular vesicle donor cell, where the at least one heterologous polynucleotide encodes a targeting polypeptide, which can increase the targeting and accumulation of the extracellular vesicle to the targeted cancer cell, tumors, non-cancer lesion cell, damaged tissue, or healthy tissue. In some cases, the extracellular vesicle donor cell is a primary cell (e.g., a primary adherent cell). In some cases, the extracellular vesicle donor cell is a cell line. In some cases, the extracellular vesicle donor cell is not genetically-modified prior to the nanoelectroporation.

Described herein are methods of treating a disease or disorder, such as cancer or tumors (e.g., malignant tumor, benign tumor) in a subject comprising systemically administering at least one extracellular vesicle to the subject. In some cases, the extracellular vesicles comprise at least one therapeutic polynucleotide (e.g., therapeutic mRNA, miRNA, etc.). In some cases, the extracellular vesicles comprising therapeutic polynucleotides can be obtained by nanoelectroporating at least one extracellular vesicle donor cell with at least a first vector and at least a second vector (e.g. a plasmid), wherein the first vector encodes tumor targeting polypeptides comprising an extracellular vesicle surface protein covalently bound to a tumor targeting domain and the second vector encodes the therapeutic polynucleotides. In some embodiments, the extracellular vesicle surface protein is CD47. In some cases, the extracellular surface protein (e.g., CD47) is covalently linked to the tumor targeting domain. In some cases, the first vectors can be expressed in the extracellular vesicle donor cells to obtain the tumor targeting polypeptides. In some instances, the second vectors can be expressed in the extracellular vesicle donor cells to obtain the therapeutic polynucleotides. In some embodiments, the extracellular vesicles released from the extracellular vesicle donor cells comprise both the tumor targeting polypeptides and the therapeutic polynucleotides. In some cases, the extracellular vesicles are collected and systematically administered to the subject. In some cases, accumulation of the extracellular vesicles with the tumor targeting polypeptides at the targeted tumor is higher compared to accumulation of extracellular vesicles lacking the tumor targeting polypeptides at the targeted tumor.

### Extracellular Vesicle Donor Cells

Described herein, in some cases, are extracellular vesicle donor cells that produce extracellular vesicles described herein. The extracellular vesicle donor cell can be any cell that can be genetically modified or manipulated to secrete extracellular vesicles at a level that is higher than the cell's basal level of secretion of extracellular vesicles. As such, a cell with low or negligible basal level of secretion of extracellular vesicles can also be an extracellular vesicle donor cell. In some cases, the extracellular vesicle donor cell can be a nucleated cell. In some cases, the extracellular vesicle donor cell can be an autologous cell. In such cases, the extracellular vesicle donor cell may be obtained from a subject; and then, following modification of the extracellular vesicle donor cell (e.g., introduction of a vector), secreted extracellular vesicles are collected and then administered to the same subject. In some cases, the extracellular vesicle donor cell is an allogeneic cell. In such case, the extracellular vesicle donor cell is a cell obtained from a source that is genetically distinct from the subject who later receives the extracellular vesicles secreted by the extracellular vesicle donor cell. Often, in the case of an allogeneic extracellular vesicle donor cell, the extracellular vesicle donor cell is of the same species, but genetically distinct from the subject who later receives the extracellular vesicles produced and secreted by the extracellular vesicle donor cell.

The extracellular vesicle donor cells can be any type of cell. In some cases, the extracellular vesicle donor cells are eukaryotic cells (e.g., mammalian cells, human cells, non-human mammalian cells, rodent cells, mouse cells, etc.). In some instances, the extracellular vesicle donor cells are cells from a cell line, stem cells, primary cells, or differentiated cells. In some embodiments, the extracellular vesicle donor cells are primary cells. In some instances, the extracellular vesicle donor cells are mouse embryonic fibroblasts (MEF), human embryonic fibroblasts (HEF), dendritic cells, mesenchymal stem cells, bone marrow-derived dendritic cells, bone marrow derived stromal cells, adipose stromal cells, enucleated cells, neural stem cells, immature dendritic cells, or immune cells. The extracellular donor cells may be adherent cells. In some cases, the extracellular vesicle donor cells are adherent cells. In some cases, the extracellular vesicle donor cells are suspension cells.

In some cases, the extracellular vesicle donor cell comprises at least one heterologous polynucleotide. In some cases, the at least one heterologous polynucleotide is introduced into the extracellular vesicle donor cell by transfection. The at least one heterologous polynucleotide can be transfected into the extracellular vesicle donor cell by any one of the biological, chemical, or physical methods described herein, or by any other biological, chemical, or physical methods. In some instances, the at least one heterologous polynucleotide is transfected into the extracellular vesicle donor cell by electroporation (e.g., nanoelectroporation). In some cases, the electroporation is microchannel electroporation or nanochannel electroporation. In some instances, the at least one heterologous polynucleotide is transfected into the extracellular vesicle donor cell by nanochannel electroporation. In some cases, the extracellular vesicle donor cells comprise genetically modified cells. Examples of genetically modified cells can include induced pluripotent stem cells or cells that are genetically modified by nucleic acid guided nuclease (e.g. CRISPR-Cas). In some cases, the extracellular vesicle donor cells are not genetically-modified. For example, in some cases, the extracellular donor cells are not genetically-modified prior to electroporation (e.g. nanoporation).

In some instances, the heterologous polynucleotide transfected into the extracellular vesicle donor cell is integrated into the chromosome of the extracellular vesicle donor cell. In some cases, the heterologous polynucleotide transfected into the extracellular vesicle donor cell is not integrated into the chromosome of the extracellular vesicle donor cell. In some cases, the extracellular vesicle donor cell is stably transfected with the heterologous polynucleotide. In some cases, the extracellular vesicle donor cell is transiently transfected with heterologous polynucleotide. In some cases, the transfected extracellular vesicle donor cell is a cell derived from a cell line. In some instances, the at least one heterologous polynucleotide is a vector (e.g. a plasmid).

In some cases, the extracellular vesicle donor cells can be electroporated by a plurality of vectors to produce and secrete extracellular vesicles. In some cases, the extracellular vesicle donor cells can be nanoelectroporated by a plurality of vectors to produce and secrete the extracellular vesicles. In some cases, the plurality of vectors comprise at least a first vector, at least a second vector, or any additional vector. In some cases, the first vectors and the second vectors can be nanoelectroporated into the extracellular vesicle donor cells at the same time. In some cases, the first vectors and the second vectors can be nanoelectroporated into the extracellular vesicle donor cells at different times. In some cases, the time difference between nanoelectroporating the first vectors and the second vectors can be at least 1 minute, 5 minutes, 10 minutes, 30 minutes, 1 hour, 5 hours, 12 hours, 1 day, 2 days, 5 days, 10 days, 30 days, or longer.

In some cases, the first vectors can encode tumor targeting polypeptides. In some instances, the extracellular vesicle donor cells, when nanoelectroporated with the first vectors, can translate the first vectors to obtain the tumor targeting polypeptides. In some cases, the extracellular vesicle donor cells can produce extracellular vesicles or exosomes comprising the tumor targeting polypeptides. In some cases, the extracellular vesicle donor cells can secrete and the produced extracellular vesicles or exosomes comprising the tumor targeting polypeptides.

In some cases, the second vectors can encode at least one therapeutic polynucleotide. In some cases, the extracellular vesicle donor cells, when nanoelectroporated with the seconds vectors, can transcribe the second vectors to obtain the therapeutic polynucleotides. In some cases, the extracellular vesicle donor cells produce and secrete the extracellular vesicles or exosomes comprising encapsulation of the therapeutic polynucleotides encoded by the second vectors. In some cases, the extracellular vesicle donor cells can produce and secrete the extracellular vesicles or exosomes comprising the tumor targeting peptide and the therapeutic polynucleotides encoded by the second vectors.

In some cases, the extracellular vesicle donor cells, when nanoelectroporated with the second vectors, can transcribe or translate the second vectors to obtain therapeutic polynucleotides or therapeutic polypeptides. In some cases, the extracellular vesicle donor cells can produce and secrete the extracellular vesicles or exosomes comprising the therapeutic polynucleotides or therapeutic polypeptides encoded by the second vectors. In some cases, the extracellular vesicle donor cells can produce and secrete the extracellular vesicles comprising the tumor targeting peptide and the therapeutic polynucleotides or therapeutic polypeptides encoded by the second vectors. In some instances, the therapeutic polynucleotides and the therapeutic polypeptides can be encapsulated in the same extracellular vesicles or exosomes. In some instances, the therapeutic polynucleotides and the therapeutic polypeptides can be encapsulated in different extracellular vesicles or exosomes.

In some cases, the extracellular vesicle donor cell continuously produces and secretes the extracellular vesicles at a steady or a basal rate. The extracellular vesicle donor cell can be any cell type, including cells that have low basal or negligible rate or production and secretion of the extracellular vesicles. For example, the extracellular vesicle donor cell can be a primary cell or a non-cancerous cell that generally do not secrete, or secrete a low number of, extracellular vesicles.

In some cases, the extracellular vesicle donor cell produces and secretes the extracellular vesicles at a basal rate. In some cases, the extracellular vesicle donor cell can be stimulated to produce and secrete extracellular vesicles at a rate that is higher than the basal rate. For example, the extracellular vesicle donor cell can be stimulated to produce and secrete extracellular vesicles at a rate that is higher than the basal rate by heat shocking the extracellular vesicle donor cell or contacting the extracellular vesicle donor cell with Ca²⁺. In some cases, the extracellular vesicle donor cell can be stimulated to produce and secrete extracellular vesicles at a rate that is higher than the basal rate by activating a stress response signaling pathway such as p53-TSAP6 signaling pathway. In some cases, the extracellular vesicle donor cell can be stimulated to produce and secrete extracellular vesicles at a rate that is higher than the basal rate by electroporating the at least one heterologous polynucleotide into the extracellular vesicle donor cell. In some cases, the extracellular vesicle donor cell can be stimulated to produce and secrete extracellular vesicles at a rate that is higher than the basal rate by microchannel electroporation or nanochannel electroporation the at least one heterologous polynucleotide into the extracellular vesicle donor cell. In some cases, the extracellular vesicle donor cell can be stimulated to produce and secrete extracellular vesicles at a rate that is higher than the basal rate by nanochannel electroporating the at least one heterologous polynucleotide into the extracellular vesicle donor cell. In some instances, the extracellular vesicle donor cell stimulated by nanochannel electroporation can produce and secrete the extracellular vesicles at a rate that is at least 0.1 fold, 0.2 fold, 0.3 fold, 0.4 fold, 0.5 fold, 0.6 fold, 0.7 fold, 0.8 fold, 0.9 fold, 2 folds, 5 folds, 10 folds, 50 folds, 100 folds, 500 folds, 1,000 folds, 5,000 folds, 10,000 fold, 50,000 folds, 100.000 fold, or more higher than the basal rate of the extracellular vesicle donor cell producing and secreting the extracellular vesicles. In some cases, the extracellular vesicle donor cell stimulated by nanochannel electroporation can produce and secrete the extracellular vesicles at a rate that is at least 0.1 fold, 0.2 fold, 0.3 fold, 0.4 fold, 0.5 fold, 0.6 fold, 0.7 fold, 0.8 fold, 0.9 fold, 2 folds, 5 folds, 10 folds, 50 folds, 100 folds, 500 folds, 1,000 folds, 5,000 folds, 10,000 fold, 50,000 folds, 100.000 fold, or more higher than the rate of the extracellular vesicle donor cell stimulated by methods other than nanoelectroporation for producing and secreting the extracellular vesicles.

In some cases, the heterologous polynucleotide transfected into the extracellular vesicle donor cell encodes at least one targeting polypeptide described herein. In some cases, the heterologous polynucleotide transfected into the extracellular vesicle donor cell encodes at least one targeting polypeptide comprising an adapter polypeptide described herein. In some instances, the adapter polypeptide comprises an extracellular domain. In some instances, the adapter polypeptide comprises a transmembrane domain. In some cases, the at least one targeting polypeptide comprises a peptide sequence of a heterologous targeting domain that is complexed to the extracellular domain of the adapter polypeptide. In some cases, the heterologous targeting domain is covalently complexed (e.g. fused) to the extracellular domain of the adapter polypeptide.

In some cases, a heterologous polynucleotide transfected into the extracellular vesicle donor cell encodes at least one therapeutic described herein. In some cases, the therapeutic is a therapeutic polynucleotide. In some instances, the therapeutic is a therapeutic polypeptide. In some instances, the extracellular vesicle donor cell transfected with at least one heterologous polynucleotide produces and secretes extracellular vesicles comprising the at least one targeting polypeptide. In some instances, the extracellular vesicle donor cell transfected with at least one heterologous polynucleotide produces and secretes extracellular vesicles comprising the at least one therapeutic. In some instances, the extracellular vesicle donor cell transfected with at least one heterologous polynucleotide produces and secretes extracellular vesicles comprising the at least one targeting polypeptide comprising an adapter polypeptide (e.g., CD47 or genetically-modified CD47) and the heterologous targeting domain that is linked to said adapter polypeptide. In some instances, the extracellular vesicle donor cell transfected with at least one heterologous polynucleotide produces and secretes extracellular vesicles comprising the at least one targeting polypeptide comprising an adapter polypeptide (e.g., CD47 or genetically-modified CD47) and the heterologous targeting domain that is linked to said adapter polypeptide and at least one therapeutic (e.g., mRNA).

### Extracellular Vesicles

Provided herein, in some cases, are compositions comprising extracellular vesicles and methods of producing extracellular vesicles. In some cases, the extracellular vesicles are any membrane-bound particle (e.g., a vesicle with a lipid bilayer). Often, the extracellular vesicles provided herein are secreted by a cell. In some instances, the extracellular vesicles are membrane-bound particles produced in vitro. In some cases, the extracellular vesicles are produced and secreted by an extracellular vesicle donor cell transfected with at least one heterologous polynucleotide. In some instances, the extracellular vesicle is an exosome, a microvesicle, a retrovirus-like particle, an apoptotic body, an apoptosome, an oncosome, an exopher, an enveloped virus, an exomere, or other very large extracellular vesicle such as a large oncosome. In some cases, the extracellular vesicle is an exosome.

In some cases, the extracellular vesicles can have a diameter about 10 nm to about 50,000 nm. In some cases, the extracellular vesicles can have a diameter about 10 nm to about 20 nm, about 10 nm to about 30 nm, about 10 nm to about 50 nm, about 10 nm to about 100 nm, about 10 nm to about 200 nm, about 10 nm to about 500 nm, about 10 nm to about 1,000 nm, about 10 nm to about 2,000 nm, about 10 nm to about 5,000 nm, about 10 nm to about 10,000 nm, about 10 nm to about 50,000 nm, about 20 nm to about 30 nm, about 20 nm to about 50 nm, about 20 nm to about 100 nm, about 20 nm to about 200 nm, about 20 nm to about 500 nm, about 20 nm to about 1,000 nm, about 20 nm to about 2,000 nm, about 20 nm to about 5,000 nm, about 20 nm to about 10,000 nm, about 20 nm to about 50,000 nm, about 30 nm to about 50 nm, about 30 nm to about 100 nm, about 30 nm to about 200 nm, about 30 nm to about 500 nm, about 30 nm to about 1,000 nm, about 30 nm to about 2,000 nm, about 30 nm to about 5,000 nm, about 30 nm to about 10,000 nm, about 30 nm to about 50,000 nm, about 50 nm to about 100 nm, about 50 nm to about 200 nm, about 50 nm to about 500 nm, about 50 nm to about 1,000 nm, about 50 nm to about 2,000 nm, about 50 nm to about 5,000 nm, about 50 nm to about 10,000 nm, about 50 nm to about 50,000 nm, about 100 nm to about 200 nm, about 100 nm to about 500 nm, about 100 nm to about 1,000 nm, about 100 nm to about 2,000 nm, about 100 nm to about 5,000 nm, about 100 nm to about 10,000 nm, about 100 nm to about 50,000 nm, about 200 nm to about 500 nm, about 200 nm to about 1,000 nm, about 200 nm to about 2,000 nm, about 200 nm to about 5,000 nm, about 200 nm to about 10,000 nm, about 200 nm to about 50,000 nm, about 500 nm to about 1,000 nm, about 500 nm to about 2,000 nm, about 500 nm to about 5,000 nm, about 500 nm to about 10,000 nm, about 500 nm to about 50,000 nm, about 1,000 nm to about 2,000 nm, about 1,000 nm to about 5,000 nm, about 1,000 nm to about 10,000 nm, about 1,000 nm to about 50,000 nm, about 2,000 nm to about 5,000 nm, about 2,000 nm to about 10,000 nm, about 2,000 nm to about 50,000 nm, about 5,000 nm to about 10,000 nm, about 5,000 nm to about 50,000 nm, or about 10,000 nm to about 50,000 nm. In some cases, the extracellular vesicles have a diameter about 10 nm, about 20 nm, about 30 nm, about 50 nm, about 100 nm, about 200 nm, about 500 nm, about 1,000 nm, about 2,000 nm, about 5,000 nm, about 10,000 nm, or about 50,000 nm. In some cases, the extracellular vesicles can have a diameter at least about 10 nm, about 20 nm, about 30 nm, about 50 nm, about 100 nm, about 200 nm, about 500 nm, about 1,000 nm, about 2,000 nm, about 5,000 nm, or about 10,000 nm. In some cases, the extracellular vesicles can have a diameter at most about 20 nm, about 30 nm, about 50 nm, about 100 nm, about 200 nm, about 500 nm, about 1,000 nm, about 2,000 nm, about 5,000 nm, about 10,000 nm, or about 50,000 nm.

In some cases, the extracellular vesicle comprises at least one targeting polypeptide. In some cases, the extracellular vesicle comprises at least one targeting polypeptide and at least one therapeutic. In some cases, the at least one targeting polypeptide comprises an adapter polypeptide comprising a transmembrane domain and an extracellular domain. In some cases, the targeting polypeptide comprises a heterologous targeting domain that is linked to the extracellular domain of the adapter polypeptide. In some cases, the adapter polypeptide comprises a peptide sequence that is at least 40%, 50%, 60%, 70%, 80%, 90%, 95%, or 99% identical to a peptide sequence of an extracellular vesicle surface protein. In some cases, the adapter polypeptide comprises a transmembrane domain of any one of the extracellular vesicle surface protein or a fragment thereof described herein. In some cases, the at least one adapter polypeptide comprises a transmembrane domain comprising a peptide sequence that is at least 40%, 50%, 60%, 70%, 80%, 90%, 95%, or 99% identical to a peptide sequence of any one of the extracellular vesicle surface protein described herein. In some cases, the at least one adapter polypeptide comprises an extracellular domain comprising a peptide sequence that is at least 40%, 50%, 60%, 70%, 80%, 90%, 95%, or 99% identical to a peptide sequence of any one of the extracellular vesicle surface protein described herein. In some cases, the targeting polypeptide is a tumor targeting polypeptide comprising a peptide sequence that is at least 40%, 50%, 60%, 70%, 80%, 90%, 95%, or 99% identical to a peptide sequence of an extracellular vesicle surface protein. In some cases, the targeting polypeptide or the tumor targeting polypeptide can be covalently bound to at least one of the targeting domain or tumor targeting domain described herein.

Extracellular vesicle surface proteins are generally proteins that are associated with extracellular vesicles. In some cases, the extracellular vesicle surface protein can be expressed by the extracellular vesicle donor cell and integrated and secreted as part of the extracellular vesicle produced and secreted by the extracellular donor cell. In some instances, the extracellular vesicle surface protein comprises at least one an extracellular domain, which can include the N-terminus, the C-terminus, or both the N and C terminus of the extracellular vesicle surface protein. In some cases, the extracellular vesicle surface protein can be encoded by the at least one heterologous polynucleotide or vector described herein. In some cases, the extracellular vesicle surface protein can be a member of the immunoglobulin superfamily Members of the immunoglobulin superfamily can include antigen receptors, antigen presenting molecules, co-receptors, antigen receptor accessory molecules, co-stimulatory or inhibitory molecules, receptors on natural killer cells, receptors on leukocytes, immunoglobulin-like cell adhesion molecules, cytokine receptors, growth factor receptors, receptor tyrosine kinases, receptor tyrosine phosphatases, immunoglobulin binding receptors, cytoskeletons, or other members. In some cases, the extracellular vesicle surface protein comprising the member of the immunoglobulin superfamily comprises a variable immunoglobulin domain (IgV) or a constant immunoglobulin domain (IgC). In some cases, the extracellular vesicle surface protein comprising the member of the immunoglobulin superfamily comprises an IgV domain. Example of the member of the immunoglobulin superfamily comprising IgV can include cluster of differentiation proteins (e.g. CD2, CD4, CD47, CD80, or CD86), myelin membrane adhesion molecules, junction adhesion molecules (JAM), tyrosine-protein kinase receptors, programmed cell death protein 1 (PD1), or T-cell antigen receptors.

In some cases, the extracellular vesicle surface protein can be modified at the N-terminus, the C-terminus, or both the N and C terminus to comprise the targeting domain described herein. Generally, extracellular vesicle proteins are transmembrane proteins (e.g., proteins that span the membrane of an extracellular vesicle) with (a) an extracellular domain; (b) a membrane spanning domain (e.g. a transmembrane domain); and/or (c) an intracellular domain. Exemplary extracellular vesicle surface protein includes 14-3-3 protein epsilon, 78 kDa glucose-regulated protein, acetylcholinesterase (AChE-S), actin, ADAM10, alkaline phosphatase, alpha-enolase, alpha-synuclein, aminopeptidase N, amyloid beta A4 (APP), annexin 5A, annexin A2, AP-1, ATF3, ATP citrate lyase, ATPase, beta actin (ACTB), beta-amyloid 42, caveolin-1, CD10, CD11a, CD11b, CD11c, CD14, CD142, CD146, CD163, CD24, CD26/DPP4, CD29/ITGB1, CD3, CD37, CD41, CD42a, CD44, CD45, CD47, CD49, CD49d, CD53, CD63, CD64, CD69, CD73, CD81, CD82, CD9, CD90, CD315, PTGFRN, claudin, cofilin-1, complement-binding proteins CD55 and CD59, cytoplasmic 1 (ACTA), cytosolic heat shock protein 90 alpha, cytosolic heat shock protein 90 beta, EBV LMP1, EBV LMP2A, EF-1alpha-1, EF2, EFGR EGFR VIII, EMMPRIN, emmprin/CD147, enolase 1 alpha (ENO1), EPCAM, ERBB2, fatty acid synthase, fetuin-A, flotillin-1, flotillin-2, fructose-bisphosphate aldolase A, glyceraldehyde-3-phosphate dehydrogenase (GAPDH), Glycophorin A, GPC1, GPI-anchored 5′nucleotidase, GTPase, heat shock protein 8 (HSPA8), heat shock proteins (HSP70 and HSP90), heparan sulfate proteoglycans, heparinase, heterotrimeric G proteins, HIV Gag, HIV Nef, HLA-DRA, HLA-G, HSV gB, HTLV-1 Tax, huntingtin, ICAM1, integrin, LAMP1/2, leucine-rich receptor kinase 2, L-lactate dehydrogenase A chain, lysosome-associated membrane glycoprotein 1, lysosome-associated membrane glycoprotein 2, MHC class I, MHC class II, MUC1, multidrug resistance-associated protein, muscle pyruvate kinase (PKM2), N-cadherin, NKCC2, PDCD6IP/Alix, PECAM1, phosphoglycerate kinase, placental prion proteins, prostate-specific antigen (PSA), pyruvate kinase (PKM), Rab-14, Rab-5a, Rab-5b, Rab-5c, Rab-7, Rap 1B, resistin, sonic hedgehog (SHH), surviving, syndecan-1, syndecan-4, syntenin-1, tetraspanins (CD9, transferrin receptor (TFR2)), TSG101, TSPAN8, tumor-associated glycoprotein tetraspanin-8, tyrosine 3 monooxygenase/tryptophan 5-monooxygenase activation protein, TYRP-2, vacuolar-sorting protein 35, zeta polypeptide (YWHAZ), or 14-3-3 protein zeta/delta. In some cases, the naturally occurring extracellular vesicle surface protein can be non-tissue specific or tissue or cell specific. In some cases, the naturally occurring extracellular vesicle surface protein is selected from the group consisting of: CD63, CD81, CD82, CD47, CD315, heterotrimeric G proteins, MHC class I, integrins, transferrin receptor (TFR2), LAMP1/2, heparan sulfate proteoglycans, EMMPRIN, ADAM10, GPI-anchored 5′nucleotidase, CD73, complement-binding proteins CD55 and CD59, and sonic hedgehog (SHH). In some cases, the naturally occurring extracellular vesicle surface protein is selected from the group consisting of: TSPAN8, CD37, CD53, CD9, PECAM1, ERBB2, EPCAM, CD90, CD45, CD41, CD42a, Glycophorin A, CD14, MHC class II, CD3, Acetylcholinesterase/AChE-S, AChE-E, amyloid beta A4/APP, PTGFRN, and multidrug resistance-associated protein.

In some cases, the at least one targeting polypeptide comprises an adapter polypeptide comprising a peptide sequence that is at least 40%, 50%, 60%, 70%, 80%, 90%, 95%, or 99% identical to a peptide sequence of CD47. In some cases, the CD47 comprises a sequence or a fragment thereof of SEQ ID NO: 1. In some cases, the CD47 comprises a transmembrane domain that corresponds to the amino acid positions of 142-162, 177-197, 208-228, 236-256, or 269-289 of SEQ ID NO: 1. In some cases, the CD47 comprises an extracellular domain that corresponds to the amino acid positions of 19-141, 198-207, or 257-268. In some cases, the CD47 comprises an extracellular domain that corresponds to the amino acid positions of 19-141. In some cases, the CD47 comprises an IgV domain that can interact with signal regulatory protein (SIRP) expressed by myeloid cells such as macrophages. Such interaction between the CD47 and the SIRP can inhibit phagocytosis activity of the myeloid cells. In some cases, the IgV domain can be part of the extracellular domain of CD47. In some cases, the adapter polypeptide described herein comprises the peptide sequence of CD47 comprising the IgV domain as part of the extracellular domain.

SEQ ID NO: 1: Human CD47, accession number:
Q08722

MWPLVAALLLGSACCGSAQLLFNKTKSVEFTFCNDTVVIPCFVTNMEAQN
TTEVYVKWKFKGRDIYTFDGALNKSTVPTDFSSAKIEVSQLLKGDASLKM
DKSDAVSHTGNYTCEVTELTREGETIIELKYRVVSWFSPNENILIVIFPI
FAILLFWGQFGIKTLKYRSGGMDEKTIALLVAGLVITVIVIVGAILFVPG
EYSLKNATGLGLIVTSTGILILLHYYVFSTAIGLTSFVIAILVIQVIAYI
LAVVGLSLCIAACIPMHGPLLISGLSILALAQLLGLVYMKFVASNQKTIQ
PPRKAVEEPLNAFKESKGMMNDE

In some cases, the adapter polypeptide comprises an extracellular domain comprising a peptide sequence that is at least 40%, 50%, 60%, 70%, 80%, 90%, 95%, or 99% identical to a peptide sequence of CD47. In some instances, the adapter polypeptide comprises a transmembrane domain comprising a peptide sequence that is at least 40%, 50%, 60%, 70%, 80%, 90%, 95%, or 99% identical to a peptide sequence of CD47. In some cases, a heterologous targeting domain is linked to the extracellular domain of the adapter polypeptide comprising the peptide sequence of CD47. In some cases, a heterologous targeting domain is linked to a N-terminus of the extracellular domain of the adapter polypeptide comprising the peptide sequence of CD47. In some cases, a heterologous targeting domain is linked to a C-terminus of the extracellular domain of the adapter polypeptide comprising the peptide sequence of CD47. In some cases, a heterologous targeting domain is covalently linked to the N-terminus of the extracellular domain of the adapter polypeptide comprising the peptide sequence of CD47. In some cases, a heterologous targeting domain is covalently linked to the C-terminus of the extracellular domain of the adapter polypeptide comprising the peptide sequence of CD47.

In some instances, the extracellular vesicle described herein comprises a plurality of targeting polypeptides comprising plurality of adapter polypeptides, where the adapter polypeptides each can comprise a peptide sequence that is at least 40%, 50%, 60%, 70%, 80%, 90%, 95%, or 99% identical to a peptide sequence of any one of extracellular vesicle surface polypeptide described herein. In some cases, the extracellular vesicle comprises a plurality of targeting polypeptides, where the plurality of the adapter polypeptides are the same. In some cases, the extracellular vesicle comprises a plurality of targeting polypeptides, where the plurality of the adapter polypeptides are different. In some cases, the extracellular vesicle comprises a plurality of targeting polypeptides, where at least one of the plurality of the adapter polypeptides comprises CD47.

In some cases, the extracellular vesicle comprising the at least one targeting polypeptide (or adapter polypeptide) exhibits increased half-life in circulation compared to half-life of an extracellular vesicle without the targeting or adapter polypeptide. In some cases, the extracellular vesicle comprising the at least one targeting polypeptide (or adapter polypeptide) comprising CD47 or a fragment thereof exhibits increased half-life in circulation compared to half-life of an extracellular vesicle without the targeting polypeptide comprising CD47 or a fragment thereof. In some cases, the half-life of the extracellular vesicle comprising the at least one targeting polypeptide comprising CD47 or a fragment thereof is increased by at least 0.1 fold, 0.2 fold, 0.5 fold, 1 fold, 2 fold, 3 fold, 5 fold, 10 fold, 20 fold, 50 fold, 100 fold, 1000 fold, or more compared to half-life of extracellular vesicle without the targeting polypeptide comprising CD47 or a fragment thereof. In some cases, the half-life of the extracellular vesicle comprising the at least one targeting polypeptide comprising CD47 or a fragment thereof is increased by at least 1 minute, 2 minutes, 3 minutes, 4 minutes, 5 minutes, 10 minutes, 15 minutes, 30 minutes, 60 minutes, 90 minutes, 2 hours, 3 hours, 4 hours, 5 hours, 6 hours, 7 hours, 8 hours, 9 hours, 10 hours, 11 hours, 12 hours, 18 hours, 24 hours, 36 hours, 48 hours, 3 days, 4 days, 5 days, 6 days, 7 days, 10 days, 12 days, 14 days, 21 days, 28 days, 30 days, or longer compared to half-life of extracellular vesicle without the targeting polypeptide comprising CD47 or a fragment thereof.

In some cases, the extracellular vesicle comprising the at least one targeting polypeptide (and/or adapter polypeptide) exhibits a half-life in circulation of a mammal (e.g, human, rodent, mouse) of at least 30 seconds, at least 1 minute, at least 2 minutes, at least 3 minutes, at least 5 minutes, or at least 10 minutes. In some cases, the extracellular vesicle comprising the at least one targeting polypeptide (and/or adapter polypeptide) comprising CD47 or a fragment thereof exhibits a half-life in circulation of a mammal (e.g, human, rodent, mouse) of at least 30 seconds, at least 1 minute, at least 2 minutes, at least 3 minutes, at least 5 minutes, or at least 10 minutes. In some cases, the extracellular vesicle comprising the targeting and/or adapter polypeptide exhibits a half-life in the circulation of a mammal of less than 5 hours, less than 2 hours, less than 1 hours, or less than 30 minutes.

In some cases, the extracellular vesicle comprises an adapter polypeptide comprising a modified CD47, where a heterologous targeting domain is attached or complexed to the extracellular domain of the adapter polypeptide. In some cases, the extracellular vesicle comprising the adapter polypeptide comprising the modified CD47 exhibits increased half-life in circulation compared to half-life of an extracellular vesicle without the adapter polypeptide comprising the CD47. In some cases, the half-life of the extracellular vesicle comprising the adapter polypeptide comprising the modified CD47 is increased by 0.1 fold, 0.2 fold, 0.5 fold, 1 fold, 2 folds, 3 folds, 5 folds, 10 folds, 20 folds, 50 folds, 100 folds, 1000 folds, or more compared to half-life of extracellular vesicle without the adapter polypeptide comprising CD47. In some cases, the half-life of the extracellular vesicle comprising the adapter polypeptide comprising the modified CD47 is increased by at least 1 minute, 2 minutes, 3 minutes, 4 minutes, 5 minutes, 10 minutes, 15 minutes, 30 minutes, 60 minutes, 90 minutes, 2 hours, 3 hours, 4 hours, 5 hours, 6 hours, 7 hours, 8 hours, 9 hours, 10 hours, 11 hours, 12 hours, 18 hours, 24 hours, 36 hours, 48 hours, 3 days, 4 days, 5 days, 6 days, 7 days, 10 days, 12 days, 14 days, 21 days, 28 days, 30 days, or longer compared to half-life of extracellular vesicle without the adapter polypeptide comprising CD47. In some cases, a rate of decrease of a number extracellular vesicles comprising the adapter polypeptide comprising the modified CD47 in circulation is decreased by 0.1 fold, 0.2 fold, 0.5 fold, 1 fold, 2 folds, 3 folds, 5 folds, 10 folds, 20 folds, 50 folds, 100 folds, 1000 folds, or more compared to a rate of decrease extracellular vesicle without the adapter polypeptide comprising CD47 in circulation, where the comparison between the extracellular vesicle with or without CD47 is made at a time interval of 1 minute, 2 minutes, 3 minutes, 4 minutes, 5 minutes, 10 minutes, 15 minutes, 30 minutes, 60 minutes, 90 minutes, 2 hours, 3 hours, 4 hours, 5 hours, 6 hours, 7 hours, 8 hours, 9 hours, 10 hours, 11 hours, 12 hours, 18 hours, 24 hours, 36 hours, 48 hours, 3 days, 4 days, 5 days, 6 days, 7 days, 10 days, 12 days, 14 days, 21 days, 28 days, 30 days, or longer.

In some instances, the extracellular vesicle comprising the adapter polypeptide comprising the modified CD47 does not exhibit reduced half-life in circulation compared to half-life of the extracellular vesicle comprising the adapter polypeptide comprising unmodified CD47. In some instances, the extracellular vesicle comprising the adapter polypeptide comprising the modified CD47 does not exhibit reduced half-life in circulation compared to half-life of the extracellular vesicle comprising the adapter polypeptide comprising unmodified CD47.

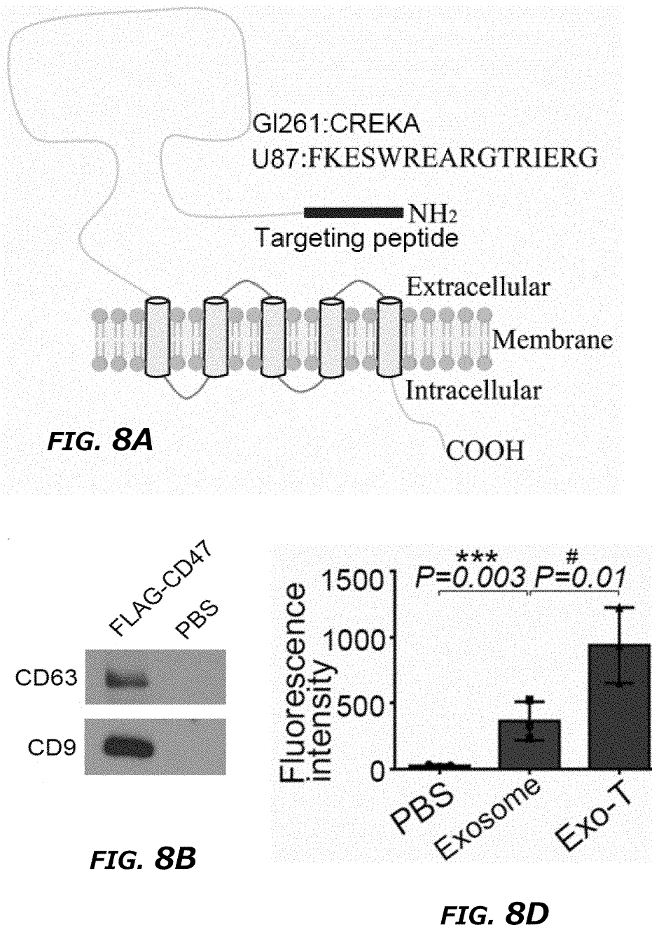

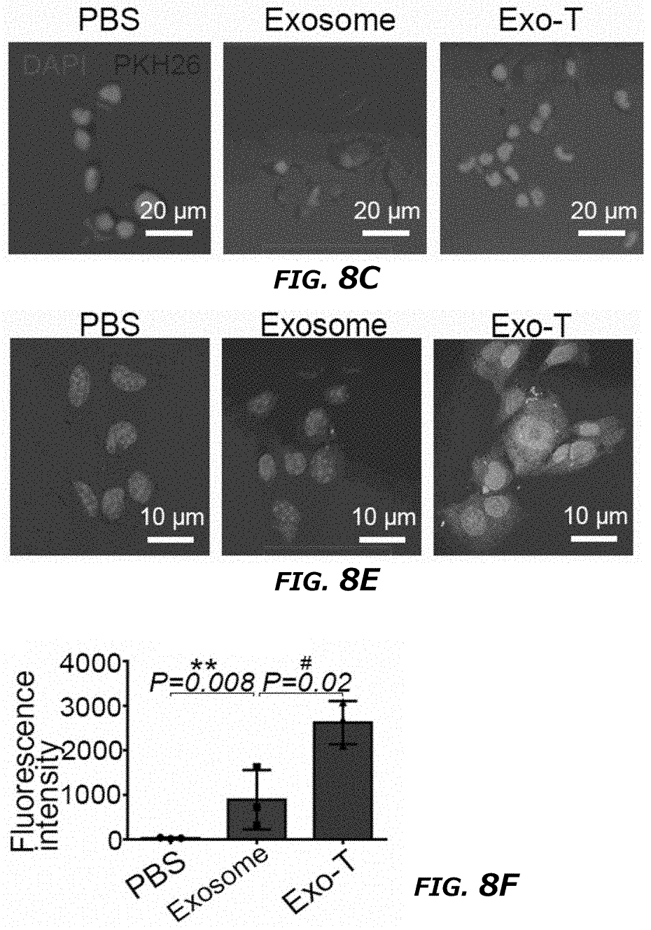

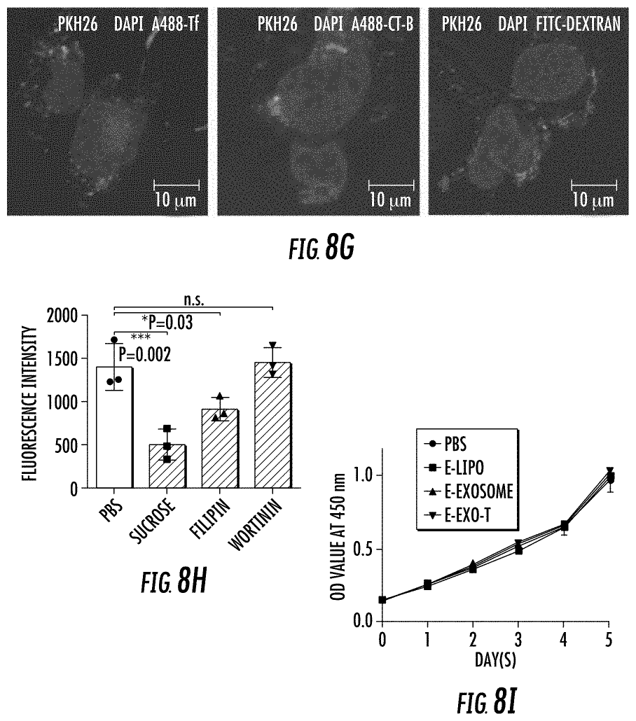

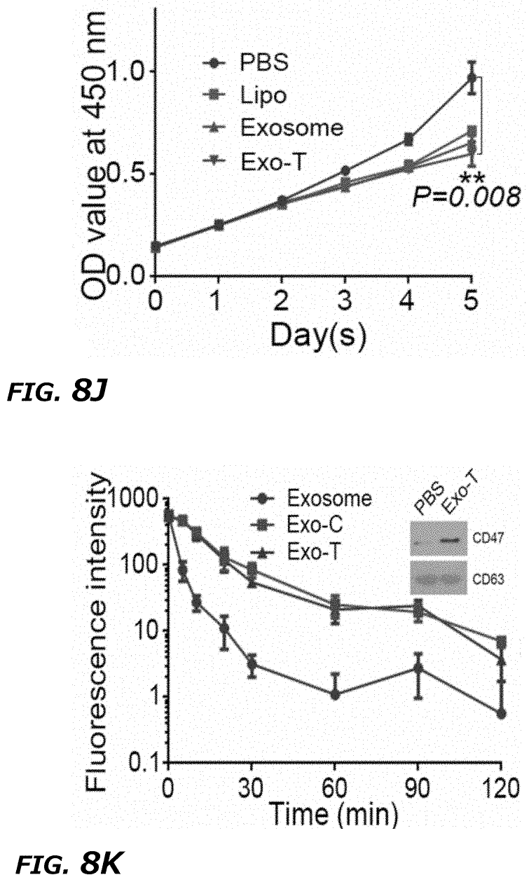

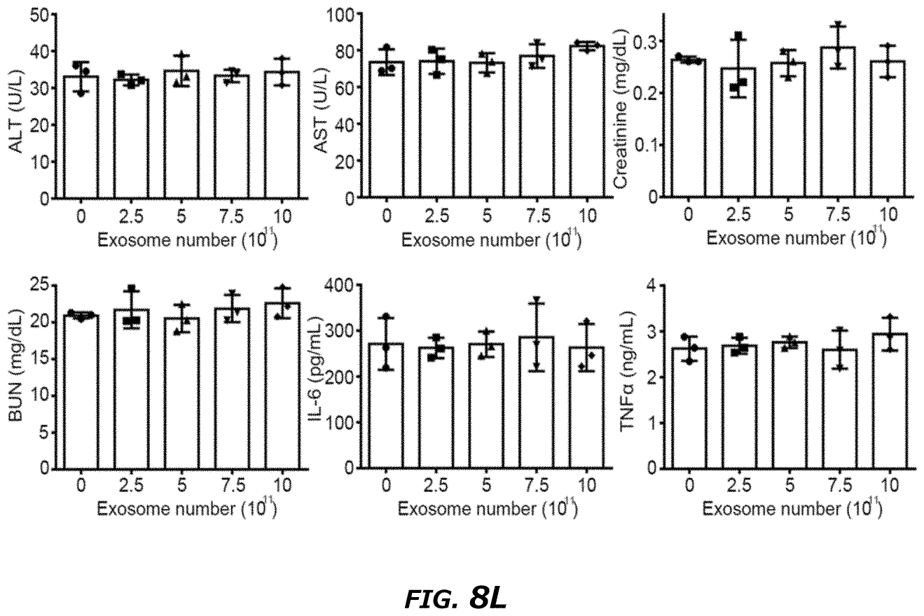

In some cases, the targeting polypeptide comprises a heterologous targeting domain. In some cases, the heterologous targeting domain is attached or complexed to the extracellular domain of the adapter polypeptide. In some cases, the heterologous targeting domain is complex to the N-terminus, C-terminus, or both N and C-terminus of the adapter polypeptide. In some instances, the heterologous targeting domain is covalently fused to the N-terminus, C-terminus, or both N and C-terminus of the adapter polypeptide. FIG. 8A illustrates an example where either a heterologous targeting domain comprising the CDX or the CREKA fused to the N-terminus extracellular domain of the adapter polypeptide comprising CD47. In some cases, the heterologous targeting domain is fused to the adapter polypeptide as part of a fusion polypeptide. In some cases, the fusion polypeptide comprising the heterologous targeting domain fused to the adapter polypeptide is encoded by the at least one heterologous polynucleotide or vector described herein.

In some instances, heterologous targeting domain is a tumor targeting domain, a tissue-targeting domain, a cell-penetrating peptide, a viral membrane protein, or a combination thereof. The heterologous targeting domain can target a cell-surface marker expressed on the surface of a targeted cell. The cell-surface marker can be any macromolecule or protein expressed on the surface of the targeted cell. Non-limiting examples of the cell-surface marker includes Vascular receptor, Fibronectin receptor, A2B5, CD44, CD24, ESA, SSEA1, CD133, CD34, CD19, CD38, CD26, CD166, or CD90.

In some instances, the accumulation of the extracellular vesicle comprising the at least one targeting polypeptide at the targeted cell expressing the cell-surface marker is higher than accumulation of extracellular vesicle without the at least one targeting polypeptide at the same targeted cell expressing the same cell-surface marker. In some instances, the accumulation of the extracellular vesicle comprising the at least one targeting polypeptide at the targeted cell expressing the cell-surface marker is at least 0.1 fold, 0.2 fold, 0.5 fold, 2 fold, 5 fold, 10 fold, 50 fold, 100 fold, 500 fold, 1,000 fold, 5,000 fold, 10,000 fold, or higher compared to the accumulation of extracellular vesicle without the targeting polypeptide at the same targeted cell expressing the same cell-surface marker.

In some instances, the hepatic and splenic accumulation, e.g. accumulation of the extracellular vesicles at non-targeted cells, of the extracellular vesicles comprising the at least one targeting polypeptide at the targeted cell expressing the cell-surface marker is reduced compared to hepatic and splenic accumulation of extracellular vesicles without the at least one targeting polypeptide at the same targeted cell. In some instances, the hepatic and splenic accumulation of the extracellular vesicles comprising the at least one targeting polypeptide is reduced by at least 0.1 fold, 0.2 fold, 0.5 fold, 2 fold, 5 fold, 10 fold, 50 fold, 100 fold, 500 fold, 1,000 fold, 5,000 fold, or 10,000 fold compared to the hepatic and splenic accumulation of extracellular vesicles without the targeting polypeptide.

In some instances, the targeting polypeptide comprises at least one heterologous targeting domain attached or complexed to the extracellular domain of the adapter polypeptide. In some cases, the at least one heterologous targeting domain is a tumor targeting domain, a tissue-targeting domain, a cell-penetrating peptide, a viral membrane protein, or a combination thereof. In some instances, the at least one heterologous targeting domain is the tumor targeting domain, where the tumor targeting domain targets a cancerous cell. In some instances, the at least one heterologous targeting domain is the tumor targeting domain, where the tumor targeting domain targets a non-cancerous lesion cell.

In some cases, the targeting polypeptide comprises at least one, two, three, four, five, or more heterologous targeting domains. In some instances, the at least two heterologous targeting domains can be identical. In some cases, the at least two heterologous targeting domains can be different. The heterologous targeting domain can be complexed to the N-terminus of the adapter polypeptide. In an alternative, the heterologous targeting domain can be complexed to the C-terminus of the adapter polypeptide. In some cases, the complexing between the heterologous targeting domain and the adapter polypeptide can be a covalent complexing. For example, the heterologous targeting domain can be covalently fused to the adapter polypeptide. In some instances, the heterologous targeting domain can be integrated into the adapter polypeptide. In some cases, the heterologous targeting domain is complexed to the adapter polypeptide via a peptide linker. In some cases, the linker peptide comprises 5 to 200 amino acids. In other cases, the linker peptide comprises 5 to 25 amino acids.

In some cases, the targeting polypeptide comprises at least one tumor targeting domain. In some cases, the targeting polypeptide comprises at least two, three, four, five, or more tumor targeting domain. In some instances, the at least two tumor targeting domain are identical. In some cases, the at least two tumor targeting domains are different. In some cases, the tumor targeting domain is fused to an N-terminus of the adapter polypeptide. In some cases, the tumor targeting domain is fused to an C-terminus of the adapter polypeptide. In some cases, the tumor targeting domain can be integrated at any peptide location of the adapter polypeptide. In some instances, the tumor targeting domain comprises at least 3, 4, 5, 6, 7, 8, 9, 10, 15, 20, 25, 30, 40, 50, or 100 amino acids. In some cases, the tumor targeting domain is a CDX (FKESWREARGTRIERG (SEQ ID NO: 2)) peptide. In some cases, the tumor targeting domain is a CREKA (SEQ ID NO: 3) peptide. In some cases, the tumor targeting domain is a CKAAKN (SEQ ID NO: 4) peptide. In some cases, the tumor targeting domain is a ARRPKLD (SEQ ID NO: 5) peptide. Other exemplary tumor targeting domain can include

In some cases, the targeting polypeptide comprises at least one tissue-targeting domain, which targets and directs the extracellular vesicle comprising the targeting polypeptide to a cell of a specific tissue. In some cases, the targeting polypeptide comprises at least two, three, four, five, or more tissue-targeting peptides. In some instances, the at least two tissue-targeting peptides are identical. In some cases, the at least two tissue-targeting peptides are different. In some cases, tissue-targeting peptide is fused to an N-terminus of the adapter polypeptide. In some cases, the tissue-targeting peptide is fused to an C-terminus of the adapter polypeptide. In some cases, the tissue-targeting peptide can be integrated at any peptide location of the adapter polypeptide. In some instances, the tissue-targeting peptide comprises at least 3, 4, 5, 6, 7, 8, 9, 10, 15, 20, 25, 30, 40, 50, or 100 amino acids. Exemplary tissue-targeting domain which targets endothelial or cardiac tissue includes SIGYPLP (SEQ ID NO: 6), LSIPPKA (SEQ ID NO: 7), FQTPPQL (SEQ ID NO: 8), LTPATAI (SEQ ID NO: 9), CNIWGVVLSWIGVFPEC (SEQ ID NO: 10), NTTTH (SEQ ID NO: 11), VHPKQHR(tetramer) (SEQ ID NO: 12), CRKRLDRNCCRTLTVRKC (SEQ ID NO: 13), CLWTVGGGC (SEQ ID NO: 14), QPWLEQAYYSTF (SEQ ID NO: 15), YPHIDSLGHWRR (SEQ ID NO: 16), LLADTTHHRPWT (SEQ ID NO: 17), SAHGTSTGVPWP (SEQ ID NO: 18), VPWMEPAYQRFL (SEQ ID NO: 19), TLPWLEESYWRP (SEQ ID NO: 20, HWRR (SEQ ID NO: 21), CSTSMLKAC (SEQ ID NO: 22), DDTRHWG (SEQ ID NO: 23), CARPAR (SEQ ID NO: 24), CKRAVR (SEQ ID NO: 25), CRSTRANPC (SEQ ID NO: 26), CPKTRRVPC (SEQ ID NO: 27), CSGMARTKC (SEQ ID NO: 28), or CRPPR (SEQ ID NO: 29). Exemplary tissue-targeting domain which targets pancreatic tissue includes CRVASVLPC (SEQ ID NO: 30), SWCEPGWCR (SEQ ID NO: 31), LSGTPERSGQAVKVKLKAIP (SEQ ID NO: 32), CHVLWSTRCCVSNPRWKC (SEQ ID NO: 33), or LSALPRT (SEQ ID NO: 34). Exemplary tissue-targeting domain which targets kidney tissue includes CLPVASC (SEQ ID NO: 35), ELRGD(R/M)AX(W/L) (SEQ ID NO: 36), GV(K/R)GX3(T/S)RDXR (SEQ ID NO: 37), HITSLLSHTTHREP (SEQ ID NO: 38), or ANTPCGPYTHDCPVKR (SEQ ID NO: 39). Exemplary tissue-targeting domain which targets lung tissue includes CGFELETCCGFECVRQCPERC (SEQ ID NO: 40), QPFMQCLCLIYDASCRNVPPIFNDVYWIAF (SEQ ID NO: 41), VNTANST (SEQ ID NO: 42), CTSGTHPRC (SEQ ID NO: 43), or SGEWVIKEARGWKHW-VFYSCCPTTPYLDITYH (SEQ ID NO: 44). Exemplary tissue-targeting domain which targets intestinal tissue includes YSGKWGW (SEQ ID NO: 45), LETTCASLCYPSYQCSYTMPHPPVVPPHPMTYSCQY (SEQ ID NO: 46), YPRLLTP (SEQ ID NO: 47), CSQSHPRHC (SEQ ID NO: 48), CSKSSDYQC (SEQ ID NO: 49), CKSTHPLSC (SEQ ID NO: 50), CTGKSCLRVG (SEQ ID NO: 51), SFKPSGLPAQSL (SEQ ID NO: 52), or CTANSSAQC (SEQ ID NO: 53). Exemplary tissue-targeting domain which targets brain tissue can include CLSSRLDAC (SEQ ID NO:54), GHKAKGPRK (SEQ ID NO: 55), HAIYPRH (SEQ ID NO: 56), THRPPMWSPVWP (SEQ ID NO: 57), HLNILSTLWKYRC (SEQ ID NO: 58), CAGALCY (SEQ ID NO: 59), CLEVSRKNC (SEQ ID NO: 60), RPRTRLHTHRNR(D-aa) (SEQ ID NO: 61), ACTTPHAWLCG (SEQ ID NO: 62), GLAHSFSDFARDFV (SEQ ID NO: 63), GYRPVHNIRGHWAPG (SEQ ID NO: 64), TGNYKALHPHNG (SEQ ID NO: 65), CRTIGPSVC (SEQ ID NO: 66), CTSTSAPYC (SEQ ID NO: 67), CSYTSSTMC (SEQ ID NO: 68), CMPRLRGC (SEQ ID NO: 69), TPSYDTYAAELR (SEQ ID NO: 70), RLSSVDSDLSGC (SEQ ID NO: 71), CAQK (SEQ ID NO: 72), or SGVYKVAYDWQH (SEQ ID NO: 73). Additional exemplary tissue-targeting domain targeting various tissue includes LMLPRAD (SEQ ID NO: 74) (targeting adrenal gland), CSCFRDVCC (SEQ ID NO: 75) (targeting retina), CRDVVSVIC (SEQ ID NO: 76) (targeting retina), CVALCREACGEGC (SEQ ID NO: 77) (targeting skin hypodermal vasculature), GLSGGRS (SEQ ID NO: 78) (targeting uterus), WYRGRL (SEQ ID NO: 79) (targeting cartilage), CPGPEGAGC (SEQ ID NO: 80) (targeting breast vasculature), SMSIARLVSFLEYR (SEQ ID NO: 81) (targeting prostate), GPEDTSRAPENQQKTGC (SEQ ID NO: 82) (targeting skin Langerhans), CKGGRAKDC (SEQ ID NO: 83) (targeting white fat vasculature), CARSKNKDC (SEQ ID NO: 84) (targeting wound or damaged tissue), CHAQGSAEC (SEQ ID NO: 85) (targeting thymus), LEPRWGFGWWLKLSTHTTESRSMV (SEQ ID NO: 86) (targeting ear or cochlea tissue), ACSTEALRHCGGGS (SEQ ID NO: 87) (targeting retinal vessel) or ASSLNIA (SEQ ID NO: 88) (targeting muscle tissue).

In some cases, the targeting polypeptide comprises at least two, three, four, five, or more cell-penetrating peptides. In some cases, the targeting polypeptide comprising the cell-penetrating peptide increases the rate of the extracellular vesicle being fused or endocytosed by the targeted cell. In some instances, the at least two cell-penetrating peptides are identical. In some cases, the at least two cell-penetrating peptides are different. In some cases, the cell-penetrating peptide is fused to an N-terminus of the adapter polypeptide. In some cases, the cell-penetrating peptide is fused to an C-terminus of the adapter polypeptide. In some cases, the cell-penetrating peptide can be integrated at any peptide location of the adapter polypeptide. In some instances, the cell-penetrating peptide comprises at least 3, 4, 5, 6, 7, 8, 9, 10, 15, 20, 25, 30, 40, 50, or 100 amino acids. Non-limiting example of the cell-penetrating peptide includes

(SEQ ID NO: 89)
DSLKSYWYLQKFSWR,
(SEQ ID NO: 90)
DWLKAFYDKVAEKLKEAF,
(SEQ ID NO: 91)
KSKTEYYNAWAVWERNAP,
(SEQ ID NO: 92)
GNGEQREMAVSRLRDCLDRQA,
(SEQ ID NO: 93)
HTPGNSNKWKHLQENKKGRPRR,
(SEQ ID NO: 94)
DWLKAFYDKVAEKLKEAF,
(SEQ ID NO: 95)
R9GPLGLAGE8,
(SEQ ID NO: 96)
Ac-GAFSWGSLWSGIKNFGSTVKNYG,
(SEQ ID NO: 97)
RLRWR,
(SEQ ID NO: 98)
LGQQQPFPPQQPY,
(SEQ ID NO: 99)
ILGKLLSTAAGLLSNL,
(SEQ ID NO: 100)
TFFYGGSRGKRNNFKTEEY,
(SEQ ID NO: 101)
Ac-LRKLRKRLLRX-Bpg-G,
(SEQ ID NO: 102)
Ac-LRKLRKRLLR,
or
(SEQ ID NO: 103)
MVRRFLVTLRIRRACGPPRVRV.

In some cases, the targeting polypeptide comprises at least two, three, four, five, or more viral membrane proteins or fragments thereof. In some cases, the targeting polypeptide comprising the viral membrane protein increases the rate of the extracellular vesicle being fused or endocytosed by the targeted cell. In some instances, the at least two viral membrane proteins are identical. In some cases, the at least two viral membrane proteins are different. In some cases, the viral membrane protein is fused to an N-terminus of the adapter polypeptide. In some cases, the viral membrane protein is fused to an C-terminus of the adapter polypeptide. In some cases, the viral membrane protein can be integrated at any peptide location of the adapter polypeptide. In some instances, the viral membrane protein comprises at least 3, 4, 5, 6, 7, 8, 9, 10, 15, 20, 25, 30, 40, 50, or 100 amino acids. Non-limiting example of the viral membrane protein includes hemagglutinin, glycoprotein 41, envelop protein, VSV G, HSVO1 gB, ebolavirus glycoprotein, or fusion-associated small transmembrane (FAST) protein.

In some cases, the extracellular vesicle described herein comprises at least one therapeutic. In some cases, the at least one therapeutic is within (e.g. encapsulated) the extracellular vesicle. In some cases, the therapeutic is a therapeutic polynucleotide. In some cases, the therapeutic is a therapeutic polypeptide. In some instances, the therapeutic is a therapeutic compound. In some cases, the therapeutic is a cancer drug comprising therapeutic polynucleotide, therapeutic polypeptide, therapeutic compound, or a combination thereof. In some instances, the extracellular vesicle comprises a plurality of therapeutics, where the plurality of therapeutics comprises therapeutic polynucleotide, therapeutic polypeptide, therapeutic compound, or a combination thereof.

In some cases, the extracellular vesicles described herein comprise at least one targeting polypeptide. In some cases, the targeting polypeptide is a tumor targeting polypeptide comprising the tumor targeting domain. In some cases, the accumulation of the extracellular vesicles comprising the tumor targeting polypeptides comprising the tumor targeting domain at the tumor is higher compared to accumulation of extracellular vesicles without the tumor target polypeptides. In some instances, the accumulation of the extracellular vesicles comprising the tumor targeting polypeptides at the tumor is at least 2 fold, 5 fold, 10 fold, 50 fold, 100 fold, 200 fold, 500 fold, 1,000 fold, 5,000 fold, or 10,000 fold higher compared to accumulation of extracellular vesicles lacking the tumor targeting polypeptide. In some instances, the accumulation of the extracellular vesicles comprising the tumor targeting polypeptides at the tumor is at least 100 fold higher compared to accumulation of extracellular vesicles lacking the tumor targeting polypeptide.

In some cases, the tumor targeting polypeptides comprise at least one tumor targeting domain In some cases, the tumor targeting domains can be on an N-terminus of the tumor targeting polypeptides. In some cases, the tumor targeting domains can be on an C-terminus of the tumor targeting polypeptides. In some cases, the tumor targeting domains can at any peptide location of the tumor targeting polypeptides. In some cases, at least two targeting domains can be on the same tumor targeting polypeptides. In some cases, the at least two targeting domains on the same tumor targeting polypeptides can be the same. In some cases, the at least two targeting domains on the same tumor targeting polypeptides can be different. In some instances, the targeting domains comprise at least 3, 4, 5, 6, 7, 8, 9, 10, 15, 20, 25, 30, 40, 50, or 100 amino acids. In some cases, the targeting domains In some cases, the tumor targeting domains can be CDX peptides. In some cases, the tumor targeting domains can be CREKA peptides.

In some cases, the extracellular vesicles comprising the extracellular vesicle surface proteins comprise increased half-life in circulation compared to half-life of extracellular vesicles without the extracellular vesicle surface proteins. In some cases, the half-life of the extracellular vesicles increased by the extracellular vesicle surface proteins is at least 90 minutes, 2 hours, 3 hours, 4 hours, 5 hours, 6 hours, 7 hours, 8 hours, 9 hours, 10 hours, 11 hours, 12 hours, 18 hours, 24 hours, 36 hours, 48 hours, 3 days, 4 days, 5 days, 6 days, 7 days, 10 days, 12 days, 14 days, 21 days, 28 days, 30 days, or longer than half-life of extracellular vesicles lacking the extracellular vesicle surface proteins.

In some cases, the extracellular vesicles comprising the extracellular vesicle surface proteins have decreased toxicity compared to the extracellular vesicles lacking the extracellular vesicle surface proteins. In such cases, often the extracellular vesicle surface proteins specifically bind to a target and do not have significant off-target binding. In some cases, the toxicity comprises toxicity to cells that are not targeted by the tumor targeting polypeptides. In some cases, the extracellular vesicles comprising the extracellular vesicle surface proteins have decreased toxicity that is at least is 1 fold, 2 fold, 3 fold, 4 fold, 5 fold, 6 fold, 7 fold, 8 fold, 9 fold, 10 fold, 20 fold, 30 fold, 50 fold, 100 fold, or more decreased compared to the extracellular vesicles lacking the extracellular vesicle surface proteins. In some cases, the decreased toxicity of the extracellular vesicles comprising the extracellular vesicle surface proteins is at least 10%, 20%, 30%, 40%, 50%, 60%, 70%, 80%, 90%, 100%, 200%, 300%, 400%, 500%, or more decreased compared to the extracellular vesicles lacking the extracellular vesicle surface proteins.

In some cases, the extracellular vesicles (e.g., exosomes) are tolerated by the subject following administration of the extracellular vesicles. For example, in some cases, the extracellular vesicles do not induce an immune response, or are not immunogenic.

### Therapeutic Polynucleotides

Described herein, in some cases, are extracellular vesicles comprising at least one therapeutic polynucleotide. In some instances, the at least one therapeutic polynucleotide is encoded by the at least one heterologous polynucleotide or vector transfected into the extracellular vesicle donor cell. In some cases, the at least one therapeutic polynucleotide comprises a peptide sequence that can be translated into a therapeutic polypeptide by the cell targeted and bound by the targeting polypeptide described herein.

In some cases, the extracellular vesicles comprise at least one therapeutic polynucleotide. In some cases, each extracellular vesicle comprises at least 1, 2, 3, 4, 5, 6, 7, 8, 9, 10, 50, 100, 500, 1,000, 5,000, 10,000, 50,000, 100,000, 500,000, 1,000,000 or more copies of the therapeutic polynucleotides. In some cases, each extracellular vesicle comprises at least 1, 2, 3, 4, 5, 6, 7, 8, 9, 10, 50, 100, 500, 1,000, 5,000, 10,000, 50,000, 100,000, 500,000, 1,000,000 or more copies of the therapeutic mRNA described herein. In some instances, the extracellular vesicles comprise at least two therapeutic polynucleotides. In some instances, the extracellular vesicles comprise at least two therapeutic polynucleotides, where the at least two therapeutic polynucleotides are different. In some cases, the at least two different therapeutic polynucleotides encapsulated by the extracellular vesicles comprise different ratio. For example, the ratio between the first and the second of the two different therapeutic polynucleotides can be 1:1,000,000, 1:500,000, 1:100,000, 1:50,000, 1:10,000, 1:5,000, 1:1,000, 1:500, 1:100, 1:50, 1:10, 1:5, 1:4, 1:3, 1:2, or 1:1. In some instances, the extracellular vesicles comprise at least two, three, four, five, six, seven, right, nine, ten or more therapeutic polynucleotides encapsulated in the same extracellular vesicle. In some cases, the extracellular vesicles can be exosomes.

In some cases, the therapeutic polynucleotides comprise mRNA, rRNA, SRP RNA, tRNA, tmRNA, snRNA, snoRNA, gRNA, aRNA, crRNA, lncRNA, miRNA, ncRNA, piRNA, siRNA, and shRNA. In some cases, the therapeutic polynucleotides comprise mRNA. In some cases, the mRNA is fully intact or substantially intact. In some cases, the mRNA encodes a portion of the protein. In some cases, the mRNA comprises at least 50, 100, 200, 500, 1,000, 5,000, 10,000, 50,000, 100,000, 500,000, or 1,000,000 of RNA nucleotides. In some instances, therapeutic polynucleotides comprise DNA. In some instances, therapeutic polynucleotides comprise DNA such as vectors that encode therapeutic polypeptide or RNA therapeutic. The therapeutic polynucleotide can encode therapeutic polypeptide including but not limited to: a tumor suppressor protein, peptide, a wild type protein counterparts of a mutant protein, a DNA repair protein, a proteolytic enzyme, proteinaceous toxin, a protein that can inhibit the activity of an intracellular protein, a protein that can activate the activity of an intracellular protein, or any protein whose loss of function needs to be reconstituted. Examples of the therapeutic polypeptide that can be encoded by the therapeutic polynucleotide (e.g. messenger RNA therapeutic) includes 123F2, Abcb4, Abcc1, Abcg2, Actb, Ada, Ahr, Akt, Akt1, Akt2, Akt3, Amhr2, Anxa7, Apc, Ar, Atm, Axin2, B2m, Bard1, Bc1211, Becn1, Bhlha15, Bin1, Blm, Braf, Brca1, Brca2, Brca3, Braf, Brcata, Brinp3, Brip1, Bub1b, Bwscr1a, Cadm3, Casc1, Casp3, Casp7, Casp8, Cav1, Ccam, Ccnd1, Ccr4, Ccs1, Cd28, Cdc25a, Cd95, Cdh1, Cdkn1a, Cdkn1b, Cdkn2a, Cdkn2b, Cdkn2c, Cftr, Chek1, Chek2, Crcs1, Crcs10, Crcs11, Crcs2, Crcs3, Crcs4, Crcs5, Crcs6, Crcs7, Crcs8, Crcs9, Ctnnb1, Cts1, Cyp1a1, Cyp2a6, Cyp2b2, Cyld, Dcc, Dkcl, Dicer1, Dmtf1, Dnmt1, Dpc4, E2f1, Eaf2, Eef1a1, Egfr, Egfr4, Erbb2, Erbb4, Ercc2, Ercc6, Ercc8, Errfi1, Esr1, Etv4, Fas1g, Fbxo10, Fcc, Fgfr3, Fntb, Foxm1, Foxn1, Fus1, Fzd6, Fzd7, Fzr1, Gadd45a, Gast, Gnai2, Gpc1, Gpr124, Gpr87, Gprc5a, Gprc5d, Grb2, Gstm1, Gstm5, Gstp1, Gstt1, H19, H2afx, Hck, Lims1, Hdac, Hexa, Hic1, Hin1, Hmmr, Hnpcc8, Hprt, Hras, Htatip2, I11b, I1110, I12, I16, I18rb Inha, Itgav, Jun, Jak3, Kit, Klf4, Kras, Kras2, Kras2b, Lig1, Lig4, Lkb1, Lmo7, Lncr1, Lncr2, Lncr3, Lncr4, Ltbp4, Luca1, Luca2, Lyz2, Lzts1, Mad111, Mad211, Madr2/Jv18, Mapk14, Mcc, Mcm4, Men1, Men2, Met, Mgat5, Mif, M1h1, M1h3, Mmac1, Mmp8, Mnt, Mpo, Msh2, Msh3, Msh6, Msmb, Mthfr, Mts1, Mutyh, Myh11, Nat2, Nbn, Ncoa3, Neil1, Nf1, Nf2, Nfe211, Nhej1, Nkx2-1, Nkx2-9, Nkx3-1, Npr12, Nqo1, Nras, Nudt1, Ogg1, Oxgr1, p16, p19, p21, p27, p27mt, p57, p14ARF, Pa1b2, Park2, Pggt1b, Pgr, Pi3k, Pik3ca, Piwil2, P16, Pla2g2a, Plg, P1k3, Pms1, Pms2, Pold1, Pole, Ppard, Pparg, Ppfia2, Ppm1d, Prdm2, Prdx1, Prkar1a, Ptch, PTEN, Prom1, Psca, Ptch1, Ptfla, Ptger2, Ptpn13, Ptprj, Rara, Rad51, Rassf1, Rb, Rb1, Rb1cc1, Rb12, Recg14, Ret, Rgs5, Rhoc, Rint1, Robo1, Rp138, S100a4, SCGB1A1, Skp2, Smad2, Smad3, Smad4, Smarcb1, Smo, Snx25, Spata13, Srpx, Ssic1, Sstr2, Sstr5, Stat3, St5, St7, St14, Stk11, Suds3, Tap1, Tbx21, Terc, Tnf, Tp53, Tp73, Trpm5, Tsc2, Tsc1, Vh1, Wrn, Wt1, Wt2, Xrcc1, Xrcc5, Xrcc6, or Zac1. In some cases, the therapeutic polynucleotide described herein can encode PTEN. In some cases, the therapeutic polypeptide encoded from the therapeutic polynucleotide described herein can be PTEN.

In some instances, a copy number of the therapeutic polynucleotide (e.g. RNA therapeutic, mRNA therapeutic) encapsulated in the extracellular vesicles is at least 1, at least 2, at least 3, at least 4, at least 5, at least 6, at least 7, at least 8, at least 9, at least 10, at least 25, at least 50, at least 100, at least 1,000, at least 10,000, at least 100,000, or more copies of the therapeutic polynucleotide per extracellular vesicle. In some instances, a copy number of the therapeutic polynucleotide (e.g. RNA therapeutic, mRNA therapeutic) encapsulated in each extracellular vesicle or exosome described herein is at least 1, at least 2, at least 3, at least 4, at least 5, at least 6, at least 7, at least 8, at least 9, at least 10, at least 25, at least 50, at least 100, at least 1,000, at least 10,000, at least 100,000, or more copies.

In some instances, a copy number of the therapeutic polynucleotide (e.g. RNA therapeutic or messenger RNA therapeutic) encapsulated in the extracellular vesicles produced from extracellular vesicle donor cell transfected by microchannel electroporating or nanochannel electroporating is increased compared to a copy number of the therapeutic polynucleotide encapsulated in the extracellular vesicles produced from extracellular vesicle donor cell transfected by other methods of transfection (e.g. conventional bulk electroporation, gene gun, lipofectamine transfection, etc) by at least 0.1 fold, 0.2 fold, 0.5 fold, 2 fold, 5 fold, 10 fold, 50 fold, 100 fold, 500 fold, 1,000 fold, 5,000 fold, 10,000 fold, or more. In some instances, a copy number of the therapeutic polynucleotide (e.g. RNA therapeutic or messenger RNA therapeutic) encapsulated in the extracellular vesicles produced from microchannel electroporated or nanochannel electroporated extracellular vesicle donor is increased by at least 0.1 fold, 0.2 fold, 0.5 fold, 2 fold, 5 fold, 10 fold, 50 fold, 100 fold, 500 fold, 1,000 fold, 5,000 fold, 10,000 fold, or more compared to a copy number of the therapeutic polynucleotide encapsulated in the extracellular vesicles produced by directly introducing the therapeutic polynucleotide into the extracellular vesicles (e.g. directly transfecting the therapeutic polynucleotide into the extracellular vesicles).

In some instances, the therapeutic polynucleotide (e.g. RNA therapeutic or messenger RNA therapeutic) encapsulated in the extracellular vesicles produced from extracellular vesicle donor cell transfected by microchannel electroporating or nanochannel electroporating is fully or substantially intact, where at least 30%, 40%, 50%, 60%, 70%, 80%, 90%, 95%, 99%, or more of the copies of the encapsulated therapeutic polynucleotide is fully intact or substantially intact. In some cases, a percentage of the fully intact or substantially intact therapeutic polynucleotide (e.g. RNA therapeutic or messenger RNA therapeutic) encapsulated in the extracellular vesicles produced from extracellular vesicle donor cell transfected by microchannel electroporating or nanochannel electroporating is increased compared to a percentage of the fully intact or substantially intact therapeutic polynucleotide (e.g. RNA therapeutic or messenger RNA therapeutic) encapsulated in the extracellular vesicles produced from extracellular vesicle donor cell transfected by other methods of transfection (e.g. conventional bulk electroporation, gene gun, lipofectamine transfection, etc). In some cases, the number of fully intact or substantially intact therapeutic polynucleotide (e.g. RNA therapeutic or messenger RNA therapeutic) encapsulated in the extracellular vesicles produced from extracellular vesicle donor cell transfected by microchannel electroporating or nanochannel electroporating is increased by at least 0.1 fold, 0.2 fold, 0.5 fold, 2 fold, 5 fold, 10 fold, 50 fold, 100 fold, 500 fold, 1,000 fold, 5,000 fold, 10,000 fold, or more compared to the number of the fully intact or substantially intact therapeutic polynucleotide encapsulated in the extracellular vesicles produced from the extracellular vesicle donor cell transfected by other methods of transfection (e.g. conventional bulk electroporation, gene gun, lipofectamine transfection, etc). In some cases, the number of the fully intact or substantially intact therapeutic polynucleotide (e.g. RNA therapeutic or messenger RNA therapeutic) encapsulated in the extracellular vesicles produced from extracellular vesicle donor cell transfected by microchannel electroporating or nanochannel electroporating is increased by at least 0.1 fold, 0.2 fold, 0.5 fold, 2 fold, 5 fold, 10 fold, 50 fold, 100 fold, 500 fold, 1,000 fold, 5,000 fold, 10,000 fold, or more compared to the number of fully intact or substantially intact therapeutic polynucleotide encapsulated in the extracellular vesicles produced from introducing the therapeutic polynucleotide directly into the extracellular vesicles (e.g. directly transfecting the therapeutic polynucleotide into the extracellular vesicles).

In some cases, the therapeutic polynucleotides comprise at least one modified nucleic acid or nucleic acid analog. Exemplary modified nucleic acids include, but are not limited to, uracil-5-yl, hypoxanthin-9-yl (I), 2-aminoadenin-9-yl, 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifiuoromethyl and other 5-substituted uracils and cytosines, 7-methylguanine and 7-methyladenine, 8-azaguanine and 8-azaadenine, 7-deazaguanine and 7-deazaadenine and 3-deazaguanine and 3-deazaadenine. Certain modified nucleic acids, such as 5-substituted pyrimidines, 6-azapyrimidines and N-2 substituted purines, N-6 substituted purines, O-6 substituted purines, 2-aminopropyladenine, 5-propynyluracil, 5-propynylcytosine, 5-methylcytosine, those that increase the stability of duplex formation, universal nucleic acids, hydrophobic nucleic acids, promiscuous nucleic acids, size-expanded nucleic acids, fluorinated nucleic acids, 5-substituted pyrimidines, 6-azapyrimidines and N-2, N-6 and O-6 substituted purines, including 2-aminopropyladenine, 5-propynyluracil and 5-propynylcytosine. 5-methylcytosine (5-me-C), 5-hydroxymethyl cytosine, xanthine, hypoxanthine, 2-aminoadenine, 6-methyl, other alkyl derivatives of adenine and guanine, 2-propyl and other alkyl derivatives of adenine and guanine, 2-thiouracil, 2-thiothymine and 2-thiocytosine, 5-halouracil, 5-halocytosine, 5-propynyl (—C≡C—CH₃) uracil, 5-propynyl cytosine, other alkynyl derivatives of pyrimidine nucleic acids, 6-azo uracil, 6-azo cytosine, 6-azo thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl, other 5-substituted uracils and cytosines, 7-methylguanine, 7-methyladenine, 2-F-adenine, 2-amino-adenine, 8-azaguanine, 8-azaadenine, 7-deazaguanine, 7-deazaadenine, 3-deazaguanine, 3-deazaadenine, tricyclic pyrimidines, phenoxazine cytidine([5,4-b][1,4]benzoxazin-2(3H)-one), phenothiazine cytidine (1H-pyrimido[5,4-b][1,4[benzothiazin-2(3H)-one), G-clamps, phenoxazine cytidine (e.g. 9-(2-aminoethoxy)-H-pyrimido[5,4-b][1,4[benzoxazin-2(3H)-one), carbazole cytidine (2H-pyrimido[4,5-b[indol-2-one), pyridoindole cytidine (H-pyrido[3′,2′:4,5[pyrrolo[2,3-d]pyrimidin-2-one), those in which the purine or pyrimidine base is replaced with other heterocycles, 7-deaza-adenine, 7-deazaguanosine, 2-aminopyridine, 2-pyridone, azacytosine, 5-bromocytosine, bromouracil, 5-chlorocytosine, chlorinated cytosine, cyclocytosine, cytosine arabinoside, 5-fluorocytosine, fluoropyrimidine, fluorouracil, 5,6-dihydrocytosine, 5-iodocytosine, hydroxyurea, iodouracil, 5-nitrocytosine, 5-bromouracil, 5-chlorouracil, 5-fluorouracil, and 5-iodouracil, 2-amino-adenine, 6-thio-guanine, 2-thio-thymine, 4-thio-thymine, 5-propynyl-uracil, 4-thio-uracil, N4-ethylcytosine, 7-deazaguanine, 7-deaza-8-azaguanine, 5-hydroxycytosine, 2′-deoxyuridine, 2-amino-2′-deoxyadenosine Modified nucleic acids comprising various heterocyclic bases and various sugar moieties (and sugar analogs) are available in the art, and the nucleic acids in some cases include one or several heterocyclic bases other than the principal five base components of naturally-occurring nucleic acids. For example, the heterocyclic base includes, in some cases, uracil-5-yl, cytosin-5-yl, adenin-7-yl, adenin-8-yl, guanin-7-yl, guanin-8-yl, 4-aminopyrrolo [2.3-d] pyrimidin-5-yl, 2-amino-4-oxopyrolo [2, 3-d] pyrimidin-5-yl, 2-amino-4-oxopyrrolo [2.3-d] pyrimidin-3-yl groups, where the purines are attached to the sugar moiety of the nucleic acid via the 9-position, the pyrimidines via the 1-position, the pyrrolopyrimidines via the 7-position and the pyrazolopyrimidines via the 1-position.

In some cases, nucleotide analogs are also modified at the phosphate moiety. Modified phosphate moieties include, but are not limited to, those with modification at the linkage between two nucleotides and contains, for example, a phosphorothioate, chiral phosphorothioate, phosphorodithioate, phosphotriester, aminoalkylphosphotriester, methyl and other alkyl phosphonates including 3′-alkylene phosphonate and chiral phosphonates, phosphinates, phosphoramidates including 3′-amino phosphoramidate and amino alkylphosphoramidates, thionophosphoramidates, thionoalkylphosphonates, thionoalkylphosphotriesters, and boranophosphates. It is understood that these phosphate or modified phosphate linkage between two nucleotides are through a 3′-5′ linkage or a 2′-5′ linkage, and the linkage contains inverted polarity such as 3′-5′ to 5′-3′ or 2′-5′ to 5′-2′. Various salts, mixed salts and free acid forms are also included.

In some cases, modified nucleic acids include 2′,3′-dideoxy-2′,3′-didehydro-nucleosides 5′-substituted DNA and RNA derivatives or 5′-substituted monomers made as the monophosphate with modified bases.

In some cases, modified nucleic acids include modifications at the 5′-position and the 2′-position of the sugar ring (, such as 5′-CH₂-substituted 2′-O-protected nucleosides. In some cases, modified nucleic acids include amide linked nucleoside dimers have been prepared for incorporation into oligonucleotides wherein the 3′ linked nucleoside in the dimer (5′ to 3′) comprises a 2′-OCH₃ and a 5′-(S)—CH₃. Modified nucleic acids can include 2′-substituted 5′-CH₂ (or O) modified nucleosides. Modified nucleic acids can include 5′-methylenephosphonate DNA and RNA monomers, and dimers. Modified nucleic acids can include 5′-phosphonate monomers having a 2′-substitution and other modified 5′-phosphonate monomers. Modified nucleic acids can include 5′-modified methylenephosphonate monomers. Modified nucleic acids can include analogs of 5′ or 6′-phosphonate ribonucleosides comprising a hydroxyl group at the 5′ and/or 6′-position. Modified nucleic acids can include 5′-phosphonate deoxyribonucleoside monomers and dimers having a 5′-phosphate group. Modified nucleic acids can include nucleosides having a 6′-phosphonate group wherein the 5′ or/and 6′-position is unsubstituted or substituted with a thio-tert-butyl group (SC(CH₃)₃) (and analogs thereof); a methyleneamino group (CH₂NH₂) (and analogs thereof) or a cyano group (CN) (and analogs thereof).

In some cases, modified nucleic acids also include modifications of the sugar moiety. In some cases, nucleic acids contain one or more nucleosides wherein the sugar group has been modified. Such sugar modified nucleosides may impart enhanced nuclease stability, increased binding affinity, or some other beneficial biological property. In certain cases, nucleic acids comprise a chemically modified ribofuranose ring moiety. Examples of chemically modified ribofuranose rings include, without limitation, addition of substituent groups (including 5′ and/or 2′ substituent groups; bridging of two ring atoms to form bicyclic nucleic acids (BNA); replacement of the ribosyl ring oxygen atom with S, N(R), or C(R₁)(R₂) (R═H, C₁-C₁₂ alkyl or a protecting group); and combinations thereof.

In some instances, a modified nucleic acid comprises modified sugars or sugar analogs. Thus, in addition to ribose and deoxyribose, the sugar moiety can be pentose, deoxypentose, hexose, deoxyhexose, glucose, arabinose, xylose, lyxose, or a sugar “analog” cyclopentyl group. The sugar can be in a pyranosyl or furanosyl form. The sugar moiety may be the furanoside of ribose, deoxyribose, arabinose or 2′-O-alkylribose, and the sugar can be attached to the respective heterocyclic bases either in [alpha] or [beta] anomeric configuration. Sugar modifications include, but are not limited to, 2′-alkoxy-RNA analogs, 2′-amino-RNA analogs, 2′-fluoro-DNA, and 2′-alkoxy- or amino-RNA/DNA chimeras. For example, a sugar modification may include 2′-O-methyl-uridine or 2′-O-methyl-cytidine. Sugar modifications include 2′-O-alkyl-substituted deoxyribonucleosides and 2′-O-ethyleneglycol like ribonucleosides. The preparation of these sugars or sugar analogs and the respective “nucleosides” wherein such sugars or analogs are attached to a heterocyclic base (nucleic acid base) is known. Sugar modifications may also be made and combined with other modifications.

Modifications to the sugar moiety include natural modifications of the ribose and deoxy ribose as well as modified modifications. Sugar modifications include, but are not limited to, the following modifications at the 2′ position: OH; F; O-, S-, or N-alkyl; O-, S-, or N-alkenyl; O-, S- or N-alkynyl; or O-alkyl-O-alkyl, wherein the alkyl, alkenyl and alkynyl may be substituted or unsubstituted C₁ to C₁₀, alkyl or C₂ to C₁₀ alkenyl and alkynyl. 2′ sugar modifications also include but are not limited to —O[(CH₂)ₙO]ₘ CH₃, —O(CH₂)ₙOCH₃, —O(CH₂)ₙNH₂, —O(CH₂)ₙCH₃, —O(CH₂)ₙONH₂, and —O(CH₂)ₙON[(CH₂)n CH₃)]₂, where n and m are from 1 to about 10.

Other modifications at the 2′ position include but are not limited to: C₁ to C₁₀ lower alkyl, substituted lower alkyl, alkaryl, aralkyl, O-alkaryl, O-aralkyl, SH, SCH₃, OCN, Cl, Br, CN, CF₃, OCF₃, SOCH₃, SO₂ CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. Similar modifications may also be made at other positions on the sugar, particularly the 3′ position of the sugar on the 3′ terminal nucleotide or in 2′-5′ linked oligonucleotides and the 5′ position of the 5′ terminal nucleotide. Modified sugars also include those that contain modifications at the bridging ring oxygen, such as CH₂ and S. Nucleotide sugar analogs may also have sugar mimetics such as cyclobutyl moieties in place of the pentofuranosyl sugar. There are numerous United States patents that teach the preparation of such modified sugar structures and which detail and describe a range of base modifications, such as U.S. Pat. Nos.

Examples of nucleic acids having modified sugar moieties include, without limitation, nucleic acids comprising 5′-vinyl, 5′-methyl (R or S), 4′-S, 2′-F, 2′-OCH₃, and 2′-O(CH₂)₂OCH₃ substituent groups. The substituent at the 2′ position can also be selected from allyl, amino, azido, thio, O-allyl, O—(C₁-C₁₀ alkyl), OCF₃, O(CH₂)₂SCH₃, O(CH₂)₂—O—N(Rₘ)(Rₙ), and O—CH₂—C(═O)—N(Rₘ)(Rₙ), where each Rₘ and Rₙ is, independently, H or substituted or unsubstituted C₁-C₁₀ alkyl.

In certain cases, nucleic acids described herein include one or more bicyclic nucleic acids. In certain such cases, the bicyclic nucleic acid comprises a bridge between the 4′ and the 2′ ribosyl ring atoms. In certain cases, nucleic acids provided herein include one or more bicyclic nucleic acids wherein the bridge comprises a 4′ to 2′ bicyclic nucleic acid. Examples of such 4′ to 2′ bicyclic nucleic acids include, but are not limited to, one of the formulae: 4′-(CH₂)—O-2′ (LNA); 4′-(CH₂)—S-2′; 4′-(CH₂)₂—O-2′ (ENA); 4′-CH(CH₃)—O-2′ and 4′-CH(CH₂OCH₃)—O-2′, and analogs thereof; 4′-C(CH₃)(CH₃)—O-2′ and analogs thereof.

In certain cases, nucleic acids comprise linked nucleic acids. Nucleic acids can be linked together using any inter nucleic acid linkage. The two main classes of inter nucleic acid linking groups are defined by the presence or absence of a phosphorus atom. Representative phosphorus containing inter nucleic acid linkages include, but are not limited to, phosphodiesters, phosphotriesters, methylphosphonates, phosphoramidate, and phosphorothioates (P═S). Representative non-phosphorus containing inter nucleic acid linking groups include, but are not limited to, methylenemethylimino (—CH₂—N(CH₃)—O—CH₂—), thiodiester (—O—C(O)—S—), thionocarbamate (—O—C(O)(NH)—S—); siloxane (—O—Si(H)₂—O—); and N,N*-dimethylhydrazine (—CH₂—N(CH₃)—N(CH₃)). In certain aspects, inter nucleic acids linkages having a chiral atom can be prepared as a racemic mixture, as separate enantiomers, e.g., alkylphosphonates and phosphorothioates. Modified nucleic acids can contain a single modification. Modified nucleic acids can contain multiple modifications within one of the moieties or between different moieties.

Backbone phosphate modifications to nucleic acid include, but are not limited to, methyl phosphonate, phosphorothioate, phosphoramidate (bridging or non-bridging), phosphotriester, phosphorodithioate, phosphodithioate, and boranophosphate, and may be used in any combination. Other non-phosphate linkages may also be used.

In some cases, backbone modifications (e.g., methylphosphonate, phosphorothioate, phosphoroamidate and phosphorodithioate internucleotide linkages) can confer immunomodulatory activity on the modified nucleic acid and/or enhance their stability in vivo.

In some instances, a phosphorous derivative (or modified phosphate group) is attached to the sugar or sugar analog moiety in and can be a monophosphate, diphosphate, triphosphate, alkylphosphonate, phosphorothioate, phosphorodithioate, phosphoramidate or the like.

In some cases, backbone modification comprises replacing the phosphodiester linkage with an alternative moiety such as an anionic, neutral or cationic group. Examples of such modifications include: anionic internucleoside linkage; N3′ to P5′ phosphoramidate modification; boranophosphate DNA; prooligonucleotides; neutral internucleoside linkages such as methylphosphonates; amide linked DNA; methylene(methylimino) linkages; formacetal and thioformacetal linkages; backbones containing sulfonyl groups; morpholino oligos; peptide nucleic acids (PNA); and positively charged deoxyribonucleic guanidine (DNG) oligos (Micklefield, 2001, Current Medicinal Chemistry 8: 1157-1179). A modified nucleic acid may comprise a chimeric or mixed backbone comprising one or more modifications, e.g. a combination of phosphate linkages such as a combination of phosphodiester and phosphorothioate linkages.

Substitutes for the phosphate include, for example, short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH₂ component parts. It is also understood in a nucleotide substitute that both the sugar and the phosphate moieties of the nucleotide can be replaced, by for example an amide type linkage (aminoethylglycine) (PNA). U.S. Pat. Nos. 5,539,082; 5,714,331; and 5,719,262 teach how to make and use PNA molecules, each of which is herein incorporated by reference. See also Nielsen et al., Science, 1991, 254, 1497-1500. It is also possible to link other types of molecules (conjugates) to nucleotides or nucleotide analogs to enhance for example, cellular uptake. Conjugates can be chemically linked to the nucleotide or nucleotide analogs. Such conjugates include but are not limited to lipid moieties such as a cholesterol moiety, cholic acid, a thioether, e.g., hexyl-S-tritylthiol, a thiocholesterol, an aliphatic chain, e.g., dodecandiol or undecyl residues (, a phospholipid, e.g., di-hexadecyl-rac-glycerol or triethylammonium l-di-O-hexadecyl-rac-glycero-S—H-phosphonate, a polyamine or a polyethylene glycol chain, or adamantane acetic acid, a palmityl moiety, or an octadecylamine or hexylamino-carbonyl-oxycholesterol moiety.

In some cases, the at least one modified nucleotide or nucleotide analogue described herein can be resistant toward nucleases such as for example ribonuclease such as RNase H, deoxyribonuclease such as DNase, or exonuclease such as 5′-3′ exonuclease and 3′-5′ exonuclease when compared to natural nucleic acid molecules. In some instances, the at least one modified nucleotide or nucleotide analogue comprises 2′-O-methyl, 2′-O-methoxyethyl (2′-O-MOE), 2′-O-aminopropyl, 2′-deoxy, T-deoxy-2′-fluoro, 2′-O-aminopropyl (2′-O-AP), 2′-O-dimethylaminoethyl (2′-O-DMAOE), 2′-O-dimethylaminopropyl (2′-O-DMAP), T-O-dimethylaminoethyloxyethyl (2′-O-DMAEOE), or 2′-O—N-methylacetamido (2′-O-NMA) modified, LNA, ENA, PNA, HNA, morpholino, methylphosphonate nucleotides, thiolphosphonate nucleotides, 2′-fluoro N3-P5′-phosphoramidites, or combinations thereof are resistant toward nucleases such as for example ribonuclease such as RNase H, deoxyribunuclease such as DNase, or exonuclease such as 5′-3′ exonuclease and 3′-5′ exonuclease. In some instances, 2′-O-methyl modified nucleic acid molecule is nuclease resistance (e.g., RNase H, DNase, 5′-3′ exonuclease or 3′-5′ exonuclease resistance). In some instances, 2′O-methoxyethyl (2′-O-MOE) modified nucleic acid molecule is nuclease resistance (e.g., RNase H, DNase, 5′-3′ exonuclease or 3′-5′ exonuclease resistance). In some instances, 2′-O-aminopropyl modified nucleic acid molecule is nuclease resistance (e.g., RNase H, DNase, 5′-3′ exonuclease or 3′-5′ exonuclease resistance). In some instances, 2′-deoxy modified nucleic acid molecule is nuclease resistance (e.g., RNase H, DNase, 5′-3′ exonuclease or 3′-5′ exonuclease resistance). In some instances, T-deoxy-2′-fluoro modified nucleic acid molecule is nuclease resistance (e.g., RNase H, DNase, 5′-3′ exonuclease or 3′-5′ exonuclease resistance). In some instances, 2′-O-aminopropyl (2′-O-AP) modified nucleic acid molecule is nuclease resistance (e.g., RNase H, DNase, 5′-3′ exonuclease or 3′-5′ exonuclease resistance). In some instances, 2′-O-dimethylaminoethyl (2′-O-DMAOE) modified nucleic acid molecule is nuclease resistance (e.g., RNase H, DNase, 5′-3′ exonuclease or 3′-5′ exonuclease resistance). In some instances, 2′-O-dimethylaminopropyl (2′-O-DMAP) modified nucleic acid molecule is nuclease resistance (e.g., RNase H, DNase, 5′-3′ exonuclease or 3′-5′ exonuclease resistance). In some instances, 2′-O-dimethylaminoethyloxyethyl (2′-O-DMAEOE) modified nucleic acid molecule is nuclease resistance (e.g., RNase H, DNase, 5′-3′ exonuclease or 3′-5′ exonuclease resistance). In some instances, 2′-O—N-methylacetamido (2′-O-NMA) modified nucleic acid molecule is nuclease resistance (e.g., RNase H, DNase, 5′-3′ exonuclease or 3′-5′ exonuclease resistance). In some instances, LNA modified nucleic acid molecule is nuclease resistance (e.g., RNase H, DNase, 5′-3′ exonuclease or 3′-5′ exonuclease resistance). In some instances, ENA modified nucleic acid molecule is nuclease resistance (e.g., RNase H, DNase, 5′-3′ exonuclease or 3′-5′ exonuclease resistance). In some instances, HNA modified nucleic acid molecule is nuclease resistance (e.g., RNase H, DNase, 5′-3′ exonuclease or 3′-5′ exonuclease resistance). In some instances, morpholinos is nuclease resistance (e.g., RNase H, DNase, 5′-3′ exonuclease or 3′-5′ exonuclease resistance). In some instances, PNA modified nucleic acid molecule is resistant to nucleases (e.g., RNase H, DNase, 5′-3′ exonuclease or 3′-5′ exonuclease resistance). In some instances, methylphosphonate nucleotides modified nucleic acid molecule is nuclease resistance (e.g., RNase H, DNase, 5′-3′ exonuclease or 3′-5′ exonuclease resistance). In some instances, thiolphosphonate nucleotides modified nucleic acid molecule is nuclease resistance (e.g., RNase H, DNase, 5′-3′ exonuclease or 3′-5′ exonuclease resistance). In some instances, nucleic acid molecule comprising 2′-fluoro N3-P5′-phosphoramidites is nuclease resistance (e.g., RNase H, DNase, 5′-3′ exonuclease or 3′-5′ exonuclease resistance). In some instances, the 5′ conjugates described herein inhibit 5′-3′ exonucleolytic cleavage. In some instances, the 3′ conjugates described herein inhibit 3′-5′ exonucleolytic cleavage.

In additional cases, the modified nucleotide or nucleotide analogue described herein is modified to increase its stability. In some embodiment, the nucleic acid molecule is RNA (e.g., mRNA). In some instances, the mRNA can be modified by one or more of the modifications to increase its stability. In some cases, the mRNA can be modified at the 2′ hydroxyl position, such as by 2′-O-methyl, 2′-O-methoxyethyl (2′-O-MOE), 2′-O-aminopropyl, 2′-deoxy, T-deoxy-2′-fluoro, 2′-O-aminopropyl (2′-O-AP), 2′-O-dimethylaminoethyl (2′-O-DMAOE), 2′-O-dimethylaminopropyl (2′-O-DMAP), T-O-dimethylaminoethyloxyethyl (2′-O-DMAEOE), or 2′-O—N-methylacetamido (2′-O-NMA) modification or by a locked or bridged ribose conformation (e.g., LNA or ENA). In some cases, the at least one modified nucleotide or nucleotide analogue is modified by 2′-O-methyl and/or 2′-O-methoxyethyl ribose. In some cases, the at least one modified nucleotide or nucleotide analogue also includes morpholinos, PNAs, HNA, methylphosphonate nucleotides, thiolphosphonate nucleotides, and/or 2′-fluoro N3-P5′-phosphoramidites to increase its stability. In some instances, the at least one modified nucleotide or nucleotide analogue is a chirally pure (or stereo pure) nucleic acid molecule. In some instances, the chirally pure (or stereo pure) nucleic acid molecule is modified to increase its stability.

In some instances, the extracellular vesicle described herein comprises at least one of any one of the therapeutic polypeptides described herein. In some cases, the at least one therapeutic polypeptide is encoded by the at least one heterologous polynucleotide or vector transfected into the extracellular vesicle donor cell.

In some cases, the therapeutic polynucleotides can be translated by the extracellular vesicle donor cells to obtain at least one therapeutic polypeptide. In some cases, the therapeutic polypeptides encoded by the therapeutic polynucleotides can be encapsulated by the extracellular vesicles produced and secreted by the extracellular vesicle donor cells. In some cases, the extracellular vesicles can encapsulate both therapeutic polynucleotides and therapeutic polypeptides encoded by the nanoelectroporated vectors. In some cases, the extracellular vesicles can be exosomes.

In some instances, the extracellular vesicle described herein can comprise at least one therapeutic compound. In some cases, the at least one therapeutic compound is complexed or anchored by any one of the extracellular vesicle surface proteins described herein. In some cases, the at least one therapeutic compound is within the extracellular vesicle. Exemplary therapeutic compounds for use in the compositions and methods described herein include therapeutic compounds which treat breast cancer, ovarian cancer, lung cancer (including non-small cell lung cancer and small cell lung cancer), pancreatic cancer, brain cancer (including brain tumors such as glioblastoma multiforme and anaplastic astrocytoma), bladder cancer, Kaposi's sarcoma, lymphoma, acute lymphocytic leukemia, and cervical cancer. Exemplary therapeutic compounds for use in the compositions and methods described herein include therapeutic compounds which are nucleoside analogs, alkylating agents, intercalating agents, and tubulin-targeting drugs. In some embodiments, the therapeutic compound for use in the compositions and methods described herein is selected from the group consisting of Gemcitabine Hydrochloride, Temozolomide, Doxorubicin, and Paclitaxel.

Treatment with Extracellular Vesicles

Described herein are methods of treating a disease in a subject by administering a therapeutic effective amount of the composition or pharmaceutical composition comprising the extracellular vesicle described herein. In some cases, the extracellular vesicle comprises the at least one targeting polypeptide and at least one therapeutic described herein. In some cases, the targeting polypeptide comprises a heterologous targeting domain comprising a tumor targeting domain, a tissue-targeting domain, a cell-penetrating peptide, a viral membrane protein, or any combination or fragment thereof. In some instances, the targeting domain respectively binds to a cell-surface marker associated with a diseased cell, where upon binding to the diseased cell the extracellular vesicle delivers the at least one therapeutic to the diseased cell. In some cases, the diseased cell is a cancer cell. In some cases, the diseased cell is a non-cancerous lesion cell. In some instances, the diseased cell is a tumor cell. In some instances, the at least one therapeutic comprises a therapeutic polynucleotide, a therapeutic polypeptide, a therapeutic compound, a cancer drug, or a combination thereof.

In some cases, targeted cell uptake of the therapeutic delivered by the extracellular vesicle comprising the at least one targeting polypeptide is increased by at least 0.1 fold, 0.2 fold, 0.5 fold, 2 fold, 5 fold, 10 fold, 50 fold, 100 fold, 500 fold, 1,000 fold, 5,000 fold, 10,000 fold, or higher compared to targeted cell uptake of the therapeutic delivered by the an extracellular vesicle without the targeting polypeptide. In some instances, the targeted cell with the increased uptake of the therapeutic delivered by the extracellular vesicle comprising the at least one targeting polypeptide is a cancerous cell, a non-cancerous lesion cell, a cell as part of a tumor, or a cell as part of a tissue.

In some cases, described herein are methods of treating a disease with the extracellular vesicle comprising targeting polypeptide and therapeutic polynucleotide described in this instant disclosure. In some cases, described herein are methods of treating a tumor with the extracellular vesicle comprising targeting polypeptide and therapeutic polynucleotide. In some cases, the methods of treating a tumor with the extracellular vesicle described herein results in inhibition of tumor growth. For example, in some cases, tumor grown may be inhibited by at least 20%, at least 30%, at least 40% or more. In some cases, the methods of treating a tumor with the extracellular vesicle described herein results in decreasing of tumor growth (e.g. death of tumor cells resulting in decreasing of the size or elimination of the tumor). In some cases, the methods of treating the tumor comprise delivering a therapeutic polynucleotide, a therapeutic polypeptide, a therapeutic compound, a cancer drug, or a combination thereof by the extracellular vesicle to the tumor cells. Non-limiting examples of the tumor cells that can be treated by the a therapeutic polynucleotide, a therapeutic polypeptide, a therapeutic compound, a cancer drug, or a combination thereof delivered by the extracellular vesicle include cells of Acanthoma, Acinic cell carcinoma, Acoustic neuroma, Acral lentiginous melanoma, Acrospiroma, Acute eosinophilic leukemia, Acute lymphoblastic leukemia, Acute megakaryoblastic leukemia, Acute monocytic leukemia, Acute myeloblastic leukemia with maturation, Acute myeloid dendritic cell leukemia, Acute myeloid leukemia, Acute promyelocytic leukemia, Adamantinoma, Adenocarcinoma, Adenoid cystic carcinoma, Adenoma, Adenomatoid odontogenic tumor, Adrenocortical carcinoma, Adult T-cell leukemia, Aggressive NK-cell leukemia, AIDS-Related Cancers, AIDS-related lymphoma, Alveolar soft part sarcoma, Ameloblastic fibroma, Anal cancer, Anaplastic large cell lymphoma, Anaplastic thyroid cancer, Angioimmunoblastic T-cell lymphoma, Angiomyolipoma, Angiosarcoma, Appendix cancer, Astrocytoma, Atypical teratoid rhabdoid tumor, Basal cell carcinoma, Basal-like carcinoma, B-cell leukemia, B-cell lymphoma, Bellini duct carcinoma, Biliary tract cancer, Bladder cancer, Blastoma, Bone Cancer, Bone tumor, Brain Stem Glioma, Brain Tumor, Breast Cancer, Brenner tumor, Bronchial Tumor, Bronchioloalveolar carcinoma, Brown tumor, Burkitt's lymphoma, Cancer of Unknown Primary Site, Carcinoid Tumor, Carcinoma, Carcinoma in situ, Carcinoma of the penis, Carcinoma of Unknown Primary Site, Carcinosarcoma, Castleman's Disease, Central Nervous System Embryonal Tumor, Cerebellar Astrocytoma, Cerebral Astrocytoma, Cervical Cancer, Cholangiocarcinoma, Chondroma, Chondrosarcoma, Chordoma, Choriocarcinoma, Choroid plexus papilloma, Chronic Lymphocytic Leukemia, Chronic monocytic leukemia, Chronic myelogenous leukemia, Chronic Myeloproliferative Disorder, Chronic neutrophilic leukemia, Clear-cell tumor, Colon Cancer, Colorectal cancer, Craniopharyngioma, Cutaneous T-cell lymphoma, Degos disease, Dermatofibrosarcoma protuberans, Dermoid cyst, Desmoplastic small round cell tumor, Diffuse large B cell lymphoma, Dysembryoplastic neuroepithelial tumor, Embryonal carcinoma, Endodermal sinus tumor, Endometrial cancer, Endometrial Uterine Cancer, Endometrioid tumor, Enteropathy-associated T-cell lymphoma, Ependymoblastoma, Ependymoma, Epithelioid sarcoma, Erythroleukemia, Esophageal cancer, Esthesioneuroblastoma, Ewing Family of Tumor, Ewing Family Sarcoma, Ewing's sarcoma, Extracranial Germ Cell Tumor, Extragonadal Germ Cell Tumor, Extrahepatic Bile Duct Cancer, Extramammary Paget's disease, Fallopian tube cancer, Fetus in fetu, Fibroma, Fibrosarcoma, Follicular lymphoma, Follicular thyroid cancer, Gallbladder Cancer, Gallbladder cancer, Ganglioglioma, Ganglioneuroma, Gastric Cancer, Gastric lymphoma, Gastrointestinal cancer, Gastrointestinal Carcinoid Tumor, Gastrointestinal Stromal Tumor, Gastrointestinal stromal tumor, Germ cell tumor, Germinoma, Gestational choriocarcinoma, Gestational Trophoblastic Tumor, Giant cell tumor of bone, Glioblastoma multiforme, Glioma, Gliomatosis cerebri, Glomus tumor, Glucagonoma, Gonadoblastoma, Granulosa cell tumor, Hairy Cell Leukemia, Hairy cell leukemia, Head and Neck Cancer, Head and neck cancer, Heart cancer, Hemangioblastoma, Hemangiopericytoma, Hemangiosarcoma, Hematological malignancy, Hepatocellular carcinoma, Hepatosplenic T-cell lymphoma, Hereditary breast-ovarian cancer syndrome, Hodgkin Lymphoma, Hodgkin's lymphoma, Hypopharyngeal Cancer, Hypothalamic Glioma, Inflammatory breast cancer, Intraocular Melanoma, Islet cell carcinoma, Islet Cell Tumor, Juvenile myelomonocytic leukemia, Kaposi Sarcoma, Kaposi's sarcoma, Kidney Cancer, Klatskin tumor, Krukenberg tumor, Laryngeal Cancer, Laryngeal cancer, Lentigo maligna melanoma, Leukemia, Leukemia, Lip and Oral Cavity Cancer, Liposarcoma, Lung cancer, Luteoma, Lymphangioma, Lymphangiosarcoma, Lymphoepithelioma, Lymphoid leukemia, Lymphoma, Macroglobulinemia, Malignant Fibrous Histiocytoma, Malignant fibrous histiocytoma, Malignant Fibrous Histiocytoma of Bone, Malignant Glioma, Malignant Mesothelioma, Malignant peripheral nerve sheath tumor, Malignant rhabdoid tumor, Malignant triton tumor, MALT lymphoma, Mantle cell lymphoma, Mast cell leukemia, Mediastinal germ cell tumor, Mediastinal tumor, Medullary thyroid cancer, Medulloblastoma, Medulloblastoma, Medulloepithelioma, Melanoma, Melanoma, Meningioma, Merkel Cell Carcinoma, Mesothelioma, Mesothelioma, Metastatic Squamous Neck Cancer with Occult Primary, Metastatic urothelial carcinoma, Mixed Mullerian tumor, Monocytic leukemia, Mouth Cancer, Mucinous tumor, Multiple Endocrine Neoplasia Syndrome, Multiple Myeloma, Multiple myeloma, Mycosis Fungoides, Mycosis fungoides, Myelodysplastic Disease, Myelodysplastic Syndromes, Myeloid leukemia, Myeloid sarcoma, Myeloproliferative Disease, Myxoma, Nasal Cavity Cancer, Nasopharyngeal Cancer, Nasopharyngeal carcinoma, Neoplasm, Neurinoma, Neuroblastoma, Neuroblastoma, Neurofibroma, Neuroma, Nodular melanoma, Non-Hodgkin Lymphoma, Non-Hodgkin lymphoma, Nonmelanoma Skin Cancer, Non-Small Cell Lung Cancer, Ocular oncology, Oligoastrocytoma, Oligodendroglioma, Oncocytoma, Optic nerve sheath meningioma, Oral Cancer, Oral cancer, Oropharyngeal Cancer, Osteosarcoma, Osteosarcoma, Ovarian Cancer, Ovarian cancer, Ovarian Epithelial Cancer, Ovarian Germ Cell Tumor, Ovarian Low Malignant Potential Tumor, Paget's disease of the breast, Pancoast tumor, Pancreatic Cancer, Pancreatic cancer, Papillary thyroid cancer, Papillomatosis, Paraganglioma, Paranasal Sinus Cancer, Parathyroid Cancer, Penile Cancer, Perivascular epithelioid cell tumor, Pharyngeal Cancer, Pheochromocytoma, Pineal Parenchymal Tumor of Intermediate Differentiation, Pineoblastoma, Pituicytoma, Pituitary adenoma, Pituitary tumor, Plasma Cell Neoplasm, Pleuropulmonary blastoma, Polyembryoma, Precursor T-lymphoblastic lymphoma, Primary central nervous system lymphoma, Primary effusion lymphoma, Primary Hepatocellular Cancer, Primary Liver Cancer, Primary peritoneal cancer, Primitive neuroectodermal tumor, Prostate cancer, Pseudomyxoma peritonei, Rectal Cancer, Renal cell carcinoma, Respiratory Tract Carcinoma Involving the NUT Gene on Chromosome 15, Retinoblastoma, Rhabdomyoma, Rhabdomyosarcoma, Richter's transformation, Sacrococcygeal teratoma, Salivary Gland Cancer, Sarcoma, Schwannomatosis, Sebaceous gland carcinoma, Secondary neoplasm, Seminoma, Serous tumor, Sertoli-Leydig cell tumor, Sex cord-stromal tumor, Sezary Syndrome, Signet ring cell carcinoma, Skin Cancer, Small blue round cell tumor, Small cell carcinoma, Small Cell Lung Cancer, Small cell lymphoma, Small intestine cancer, Soft tissue sarcoma, Somatostatinoma, Soot wart, Spinal Cord Tumor, Spinal tumor, Splenic marginal zone lymphoma, Squamous cell carcinoma, Stomach cancer, Superficial spreading melanoma, Supratentorial Primitive Neuroectodermal Tumor, Surface epithelial-stromal tumor, Synovial sarcoma, T-cell acute lymphoblastic leukemia, T-cell large granular lymphocyte leukemia, T-cell leukemia, T-cell lymphoma, T-cell prolymphocytic leukemia, Teratoma, Terminal lymphatic cancer, Testicular cancer, Thecoma, Throat Cancer, Thymic Carcinoma, Thymoma, Thyroid cancer, Transitional Cell Cancer of Renal Pelvis and Ureter, Transitional cell carcinoma, Urachal cancer, Urethral cancer, Urogenital neoplasm, Uterine sarcoma, Uveal melanoma, Vaginal Cancer, Verner Morrison syndrome, Verrucous carcinoma, Visual Pathway Glioma, Vulvar Cancer, Waldenstrom's macroglobulinemia, Warthin's tumor, Wilms' tumor, and combinations thereof. In some cases, the cancer cell targeted by the extracellular vesicles represents a subpopulation within a cancer cell population, such as a cancer stem cell.

In some cases, described herein are methods of treating a muscle disease by administering the extracellular vesicle comprising targeting polypeptide and therapeutic polynucleotide described in this instant disclosure to the subject with the muscle disease. In some cases, described herein are methods of treating a muscular dystrophy in the subject with the extracellular vesicle comprising muscle cell targeting polypeptide and therapeutic polynucleotide. In some cases, the muscular dystrophy is selected from the group consisting of: Duchenne muscular dystrophy, Becker muscular dystrophy, facioscapulohumeral muscular dystrophy, congenital muscular dystrophy, and myotonic dystrophy. In some cases, the therapeutic polynucleotide delivered to the muscle cells comprises mRNA encoding full length or truncated protein. In some cases, the therapeutic polynucleotide delivered to the muscle cells comprise anti-sense oligonucleotides that induce skipping of exon of a protein.

In some cases, described herein are methods of treating an ophthalmological disease by administering the extracellular vesicle comprising targeting polypeptide and therapeutic polynucleotide described in this instant disclosure to the subject. In some cases, the described herein are methods of treating an ophthalmological disease in the subject with the extracellular vesicle comprising ophthalmological cell targeting polypeptide and therapeutic polynucleotide. In some instances, the ophthalmological disease is a retinal disease. In some cases, the retinal disease is retinitis pigmentosa. In some instances, the retinal disease is Leber's congenital amaurosis. In some instances, described herein are methods of treating retinal diseases with therapeutic polynucleotide delivered to retinal cells by the extracellular vesicle described in this instant disclosure.

In some cases, described herein methods of treating a disease by administering the extracellular vesicle comprising targeting polypeptide and therapeutic polynucleotide to a subject in need thereof. In some cases, the extracellular vesicle comprising targeting polypeptide and therapeutic polynucleotide is administered daily, every day, every alternate day, five days a week, once a week, every other week, two weeks per month, three weeks per month, once a month, twice a month, three times per month, or more. The extracellular vesicle comprising targeting polypeptide and therapeutic polynucleotide can be administered for at least 1 month, 2 months, 3 months, 4 months, 5 months, 6 months, 7 months, 8 months, 9 months, 10 months, 11 months, 12 months, 18 months, 2 years, 3 years, or more.

In the case wherein the subject's status improves, the dose of the extracellular vesicle comprising targeting polypeptide and therapeutic polynucleotide being administered can be temporarily reduced or temporarily suspended for a certain length of time (a “drug holiday”). In some instances, the length of the drug holiday varies between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, or 365 days. The dose reduction during a drug holiday can be from 10%-100%, including, by way of example only, 10%, 15%, 20%, 25%, 30%, 35%, 40%, 45%, 50%, 55%, 60%, 65%, 70%, 75%, 80%, 85%, 90%, 95%, or 100%.

In some cases, an effective amount of the extracellular vesicle comprising targeting polypeptide and therapeutic polynucleotide can be administered to a subject in need thereof once per week, once every two weeks, once every three weeks, once every 4 weeks, once every 5 weeks, once every 6 weeks, once every 7 weeks, once every 8 weeks, once every 9 weeks, once every 10 weeks, once every 11 weeks, once every 12 weeks, once every 13 weeks, once every 14 weeks, once every 15 weeks, once every 16 weeks, once every 17 weeks, once every 18 weeks, once every 19 weeks, once every 20 weeks, once every 21 weeks, once every 22 weeks, once every 23 weeks, once every 24 weeks, once every 25 weeks, once every 26 weeks, once every 27 weeks, or once every 28 weeks.

Once improvement of the subject's disease or condition associated with the disease have occurred, a maintenance dose of extracellular vesicles is administered if necessary. Subsequently, the dosage or the frequency of administration, or both, can be reduced, as a function of the symptoms, to a level at which the improved disease, disorder or condition is retained.

In some cases, the amount of the extracellular vesicle comprising targeting polypeptide and therapeutic polynucleotide that correspond to such an amount varies depending upon factors such as the severity of the disease, the identity (e.g., weight) of the subject or host in need of treatment, but nevertheless is routinely determined in a manner known in the art according to the particular circumstances surrounding the case, including, e.g., the specific extracellular vesicle being administered, the route of administration, and the subject or host being treated. In some instances, the desired dose is conveniently presented in a single dose or as divided doses administered simultaneously (or over a short period of time) or at appropriate intervals, for example as two, three, four or more sub-doses per day.

In some cases, the dosage can be at least partially determined by occurrence or severity of grade 3 or grade 4 adverse events in the subject. Non-limiting examples of adverse events include hypothermia; shock; bradycardia; ventricular extrasystoles; myocardial ischemia; syncope; hemorrhage; atrial arrhythmia; phlebitis; atrioventricular (AV) block second degree; endocarditis; pericardial effusion; peripheral gangrene; thrombosis; coronary artery disorder; stomatitis; nausea and vomiting; liver function tests abnormal; gastrointestinal hemorrhage; hematemesis; bloody diarrhea; gastrointestinal disorder; intestinal perforation; pancreatitis; anemia; leukopenia; leukocytosis; hypocalcemia; alkaline phosphatase increase; blood urea nitrogen (BUN) increase; hyperuricemia; non-protein nitrogen (NPN) increase; respiratory acidosis; somnolence; agitation; neuropathy; paranoid reaction; convulsion; grand mal convulsion; delirium; asthma, lung edema; hyperventilation; hypoxia; hemoptysis; hypoventilation; pneumothorax; mydriasis; pupillary disorder; kidney function abnormal; kidney failure; acute tubular necrosis; duodenal ulceration; bowel necrosis; myocarditis; supraventricular tachycardia; permanent or transient blindness secondary to optic neuritis; transient ischemic attacks; meningitis; cerebral edema; pericarditis; allergic interstitial nephritis; tracheo-esophageal fistula; malignant hyperthermia; cardiac arrest; myocardial infarction; pulmonary emboli; stroke; liver or renal failure; severe depression leading to suicide; pulmonary edema; respiratory arrest; respiratory failure; leukopenia, thrombocytopenia, increased alanine aminotransferase (ALT), anorexia, arthralgia, back pain, chills, diarrhea, dyslipidemia, fatigue, fever, flu-like symptoms, hypoalbuminemia, increased lipase, injection site reaction, myalgia, nausea, night sweats, pruritis, rash, erythematous rash, maculopapular rash, transaminitis, vomiting, and weakness.

The foregoing ranges are merely suggestive, as the number of variables in regard to an individual treatment regime is large, and considerable excursions from these recommended values are not uncommon. Such dosages are altered depending on a number of variables, not limited to the activity of the compound used, the disease or condition to be treated, the mode of administration, the requirements of the individual subject, the severity of the disease or condition being treated, and the judgment of the practitioner.

In some cases, toxicity and therapeutic efficacy of such therapeutic regimens are determined by standard pharmaceutical procedures in cell cultures or experimental animals, including, but not limited to, the determination of the LD50 (the dose lethal to 50% of the population) and the ED50 (the dose therapeutically effective in 50% of the population). The dose ratio between the toxic and therapeutic effects is the therapeutic index and it is expressed as the ratio between LD50 and ED50. Compounds exhibiting high therapeutic indices are preferred. The data obtained from cell culture assays and animal studies are used in formulating a range of dosage for use in human. The dosage of such compounds lies preferably within a range of circulating concentrations that include the ED50 with minimal toxicity. The dosage varies within this range depending upon the dosage form employed and the route of administration utilized.

### Production of Extracellular Vesicles

Described herein, in some cases, are methods and systems of producing the extracellular vesicles comprising the targeting polypeptide the therapeutic polypeptide, the therapeutic compound, the cancer drug, or a combination thereof.

In some cases, the method comprises introducing at least one heterologous polynucleotide into an extracellular vesicle donor cell. In some cases, the at least one heterologous polynucleotide is a vector. In some instances, the at least one heterologous polynucleotide introduced into the extracellular vesicle donor cells encodes at least one targeting polypeptide described herein. In some cases, the at least one heterologous polynucleotide encodes at least one heterologous targeting domain. In some instances, the at least one heterologous polynucleotide comprises at least therapeutic polynucleotide described herein. In some instances, the at least one heterologous polynucleotide encodes at least one therapeutic polynucleotide described herein. In some instances, the at least one heterologous polynucleotide encodes at least one therapeutic polypeptide described herein.

In some instances, at least two heterologous polynucleotides are introduced into an extracellular donor cell, where a first heterologous polynucleotide comprising a first vector encoding at least one targeting polypeptide or tumor targeting polypeptide. In some cases, a second heterologous polynucleotide introduced into the extracellular vesicle donor cell comprises a second vector encoding the at least one therapeutic polynucleotide or the at least one therapeutic polypeptide.

In some cases, the heterologous polynucleotide can be introduced into the cell via the use of expression vectors. In the context of an expression vector, the vector can be readily introduced into the cell described herein by any method in the art. For example, the expression vector can be transferred into the cell by biological, chemical, or physical methods. In some cases, the extracellular vesicle donor cell can be any type of cell described herein. In some cases, the extracellular donor cell can be nucleated cell.

Biological methods for introducing the heterologous polynucleotide of interest into the cell can include the use of DNA or RNA vectors. Viral vectors, and especially retroviral vectors, have become the most widely used method for inserting genes into non-human mammalian cells. Other viral vectors, in some cases, are derived from lentivirus, poxviruses, herpes simplex virus I, adenoviruses and adeno-associated viruses, and the like. Exemplary viral vectors include retroviral vectors, adenoviral vectors, adeno-associated viral vectors (AAVs), pox vectors, parvoviral vectors, baculovirus vectors, measles viral vectors, or herpes simplex virus vectors (HSVs). In some instances, the retroviral vectors include gamma-retroviral vectors such as vectors derived from the Moloney Murine Keukemia Virus (MoMLV, MMLV, MuLV, or MLV) or the Murine Steam cell Virus (MSCV) genome. In some instances, the retroviral vectors also include lentiviral vectors such as those derived from the human immunodeficiency virus (HIV) genome. In some instances, AAV vectors include AAV1, AAV2, AAV4, AAV5, AAV6, AAV7, AAV8, or AAV9 serotype. In some instances, viral vector is a chimeric viral vector, comprising viral portions from two or more viruses. In additional instances, the viral vector is a recombinant viral vector.

Chemical methods for introducing the heterologous polynucleotide into the cell can include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle). Other methods of state-of-the-art targeted delivery of nucleic acids are available, such as delivery of polynucleotides with targeted nanoparticles or other suitable sub-micron sized delivery system.

In the case where a non-viral delivery system is utilized, an exemplary delivery vehicle is a liposome. The use of lipid formulations is contemplated for the introduction of the nucleic acids into a host cell (in vitro, ex vivo or in vivo). In another aspect, the nucleic acid is associated with a lipid. The nucleic acid associated with a lipid, in some cases, is encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution. For example, in some cases, they are present in a bilayer structure, as micelles, or with a “collapsed” structure. Alternately, they are simply be interspersed in a solution, possibly forming aggregates that are not uniform in size or shape. Lipids are fatty substances which are, in some cases, naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes.

Lipids suitable for use are obtained from commercial sources. For example, in some cases, dimyristyl phosphatidylcholine (“DMPC”) is obtained from Sigma, St. Louis, Mo.; in some cases, dicetyl phosphate (“DCP”) is obtained from K & K Laboratories (Plainview, N.Y.); cholesterol (“Choi”), in some cases, is obtained from Calbiochem-Behring; dimyristyl phosphatidylglycerol (“DMPG”) and other lipids are often obtained from Avanti Polar Lipids, Inc. (Birmingham, Ala.). Stock solutions of lipids in chloroform or chloroform/methanol are often stored at about −20° C. Chloroform is used as the only solvent since it is more readily evaporated than methanol. “Liposome” is a generic term encompassing a variety of single and multilamellar lipid vehicles formed by the generation of enclosed lipid bilayers or aggregates. Liposomes are often characterized as having vesicular structures with a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers. However, compositions that have different structures in solution than the normal vesicular structure are also encompassed. For example, the lipids, in some cases, assume a micellar structure or merely exist as nonuniform aggregates of lipid molecules. Also contemplated are lipofectamine-nucleic acid complexes.

Physical methods for introducing the heterologous polynucleotide into the cell can include calcium phosphate precipitation, lipofection, particle bombardment, microinjection, gene gun, electroporation, micro-needle array, nano-needle array, sonication, or chemical permeation. Electroporation includes microfluidics electroporation, microchannel electroporation, or nanochannel electroporation. In certain cases, the extracellular vesicle donor cell is transfected with the at least one heterologous polynucleotide by microchannel electroporation or nanochannel electroporation. In some instances, the microchannel electroporation or the nanochannel electroporation comprises use of micropore patterned silicon wafers, nanopore patterned silicon wafers, track etch membranes, ceramic micropore membranes, ceramic nanopore membranes, other porous materials, or a combination thereof. In some instances, the at least one heterologous polynucleotide or the at least one vector is nanoelectroporated into the extracellular vesicle donor cell via a nanochannel located on a biochip.

In some cases, extracellular vesicle donor cells can be grown and attached on a surface of a substrate. In some cases, the substrate comprises a biochip. In some cases, the surface of the substrate comprise metallic material. In some cases, the substrates comprise metallic material. Non-limiting examples of metallic material include aluminum (Al), indium tin oxide (ITO, In₂O₃:SnO2), chromium (Cr), gallium arsenide (GaAs), gold (Au), molybdenum (Mo), organic residues and photoresist, platinum (Pt), silicon (Si), silicon dioxide (SiO₂), silicon on insulator (SOI), silicon nitride (Si₃N₄) tantalum (Ta), titanium (Ti), titanium nitride (TiN), tungsten (W). In some cases, the metallic material can be treated or etched to create an array or channels. In some cases, the metallic surface can be treated or etched with phosphoric acid (H₃PO₄), acetic acid, nitric acid (HNO₃), water (H₂O), hydrochloric acid (HCl), (HNO₃), ceric ammonium nitrate ((NH₄)₂Ce(NO₃)₆, citric acid (C₆H₈O₇), hydrogen peroxide (H₂O₂), aqua regia, iodine solution, sulfuric acid (H₂SO₄), hydrofluoric acid (HF), potassium hydroxide (KOH), ethylenediamine pyrocatechol (EDP), tetramethylammonium hydroxide (TMAH), buffered oxide, ammonium fluoride (NH₄F), SCl, Cl₂, CCl₄, SiCl₄, BCl₃, SiCl₄, BCl₃, CCl₂F₂, CF₄, O₂, CF₄, SF₆, NF₃, CHF₃, or a combination thereof.

In some cases, the metallic surface can be treated with a gas or plasma to increase hydrophilicity. In some cases, the metallic surface can be treated with a gas or plasma to increase hydrophobicity. Exemplary gas or plasma for increasing hydrophilicity or hydrophobicity of the metallic surface include oxygen, nitrogen, ammonia, argon, chlorine, fluorine, bromine, iodine, astatine, hydrogen, or a combination thereof.

In some cases, the extracellular vesicle donor cells can be grown and attached to a surface of a substrate made of polymers such as polypropylene, polyethylene, polystyrene, ABS, polyamide, polyethylene copolymer, epoxy, polyester, polyvinylchloride, phenolic, polytetrafluoroethylene, polyethylene copolymer, fluorinated ethylene propylene, polyvinylidene, silicone, natural rubber, latex, polyurethane, styrene butadiene rubber, fluorocarbon copolymer elastomer, polyethylene terephthalate, polycarbonate, polyamide, polyaramid, polyaryl ether ketone, polyacetal, polyphenylene oxide, PBT, polysulfone, polyethersulfone, polyarylsulfone, polyphenylene sulfide, polytetrafluoroethylene, beryllium oxide etc. In some cases, the surface made of polymers can be semi-permeable. In some embodiment, pore size of the semi-permeable polymer surface can be between about 0.01 μm to about 10 μm. In some embodiment, pore size of the semi-permeable polymer surface can be between about 0.01 μm to about 0.03 μm, about 0.01 μm to about 0.05 μm, about 0.01 μm to about 0.1 μm, about 0.01 μm to about 0.2 μm, about 0.01 μm to about 0.3 μm, about 0.01 μm to about 0.4 μm, about 0.01 μm to about 0.5 μm, about 0.01 μm to about 1 μm, about 0.01 μm to about 3 μm, about 0.01 μm to about 5 μm, about 0.01 μm to about 10 μm, about 0.03 μm to about 0.05 μm, about 0.03 μm to about 0.1 μm, about 0.03 μm to about 0.2 μm, about 0.03 μm to about 0.3 μm, about 0.03 μm to about 0.4 μm, about 0.03 μm to about 0.5 μm, about 0.03 μm to about 1 μm, about 0.03 μm to about 3 μm, about 0.03 μm to about 5 μm, about 0.03 μm to about 10 μm, about 0.05 μm to about 0.1 μm, about 0.05 μm to about 0.2 μm, about 0.05 μm to about 0.3 μm, about 0.05 μm to about 0.4 μm, about 0.05 μm to about 0.5 μm, about 0.05 μm to about 1 μm, about 0.05 μm to about 3 μm, about 0.05 μm to about 5 μm, about 0.05 μm to about 10 μm, about 0.1 μm to about 0.2 μm, about 0.1 μm to about 0.3 μm, about 0.1 μm to about 0.4 μm, about 0.1 μm to about 0.5 μm, about 0.1 μm to about 1 μm, about 0.1 μm to about 3 μm, about 0.1 μm to about 5 μm, about 0.1 μm to about 10 μm, about 0.2 μm to about 0.3 μm, about 0.2 μm to about 0.4 μm, about 0.2 μm to about 0.5 μm, about 0.2 μm to about 1 μm, about 0.2 μm to about 3 μm, about 0.2 μm to about 5 μm, about 0.2 μm to about 10 μm, about 0.3 μm to about 0.4 μm, about 0.3 μm to about 0.5 μm, about 0.3 μm to about 1 μm, about 0.3 μm to about 3 μm, about 0.3 μm to about 5 μm, about 0.3 μm to about 10 μm, about 0.4 μm to about 0.5 μm, about 0.4 μm to about 1 μm, about 0.4 μm to about 3 μm, about 0.4 μm to about 5 μm, about 0.4 μm to about 10 μm, about 0.5 μm to about 1 μm, about 0.5 μm to about 3 μm, about 0.5 μm to about 5 μm, about 0.5 μm to about 10 μm, about 1 μm to about 3 μm, about 1 μm to about 5 μm, about 1 μm to about 10 μm, about 3 μm to about 5 μm, about 3 μm to about 10 μm, or about 5 μm to about 10 μm. In some embodiment, pore size of the semi-permeable polymer surface can be between about 0.01 μm, about 0.03 μm, about 0.05 μm, about 0.1 μm, about 0.2 μm, about 0.3 μm, about 0.4 μm, about 0.5 μm, about 1 μm, about 3 μm, about 5 μm, or about 10 μm. In some embodiment, pore size of the semi-permeable polymer surface can be between at least about 0.01 μm, about 0.03 μm, about 0.05 μm, about 0.1 μm, about 0.2 μm, about 0.3 μm, about 0.4 μm, about 0.5 μm, about 1 μm, about 3 μm, or about 5 μm. In some embodiment, pore size of the semi-permeable polymer surface can be between at most about 0.03 μm, about 0.05 μm, about 0.1 μm, about 0.2 μm, about 0.3 μm, about 0.4 μm, about 0.5 μm, about 1 μm, about 3 μm, about 5 μm, or about 10 μm.

In some cases, the surface of the polymer can be treated with a gas or plasma to increase hydrophilicity. In some cases, the surface of the polymer can be treated with a gas or plasma to increase hydrophobicity. Exemplary gas or plasma for increasing hydrophilicity or hydrophobicity of the metallic surface include oxygen, nitrogen, ammonia, argon, chlorine, fluorine, bromine, iodine, astatine, hydrogen, or a combination thereof.

### Nanoelectroporation

In some cases, the extracellular vesicle donor cells grown or attached to a metallic or polymer surface can be nanoelectroporated by nanoelectroporation systems as described herein. In some cases, the extracellular vesicle donor cells to be nanoelectroporated by nanoelectroporation systems described herein can be grown or attached to the metallic or polymer surface such as the biochip described herein. In some cases, the extracellular vesicle donor cells to be nanoelectroporated by nanoelectroporation systems described herein can be grown or attached to the metallic or polymer surface such as the biochip described herein in a monolayer. In some cases, the systems comprise a fluidic chamber with an upper boundary and a lower boundary. The placement of the substrate with the extracellular vesicle donor cells in the fluid chamber create an upper chamber and a lower chamber. In some cases, the systems further comprise at least one nanochannel. In some cases, the nanochannels can be embedded within the substrate.

In some cases, the extracellular vesicle donor cells grown or attached to a metallic or polymer surface and nanoelectroporated with the heterologous polynucleotide described herein can result in high-throughput production of extracellular vesicles (e.g. exosomes). In some cases, such high-throughput production of exosomes can involve use of a plurality (e.g., greater than 1, greater than 2, greater than 3, greater than 5, greater than 10, or additional numbers) biochips (e.g., CNP biochips). In some cases, the CNP biochip comprises a width that is at least 1 cm, 2 cm, 3 cm, 4 cm, 5 cm, 6 cm, 7 cm, 8 cm, 9 cm, 10 cm, 15 cm, 20 cm, 25 cm, 30 cm, 35 cm, 40 cm, 45 cm, 50 cm, 100 cm, or more cm. In some cases, the CNP biochip comprises a length that is at least 1 cm, 2 cm, 3 cm, 4 cm, 5 cm, 6 cm, 7 cm, 8 cm, 9 cm, 10 cm, 15 cm, 20 cm, 25 cm, 30 cm, 35 cm, 40 cm, 45 cm, 50 cm, 100 cm, or more cm. In some cases, the biochip comprises a dimension of 1 cm×1 cm. In some cases, the biochip can comprise exemplary dimensions of 1 cm×2 cm, 1 cm×3 cm, 1 cm×5 cm, 1 cm×10 cm, 2 cm×1 cm, 2 cm×2 cm, 2 cm×3 cm, 2 cm×5 cm, 2 cm×10 cm, 3 cm×1 cm, 3 cm×2 cm, 3 cm×3 cm, 3 cm×5 cm, 3 cm×10 cm, 5 cm×1 cm, 5 cm×2 cm, 5 cm×3 cm, 5 cm×5 cm, 5 cm×10 cm, 10 cm×1 cm, 10 cm×2 cm, 10 cm×3 cm, 10 cm×5 cm, or 10 cm×10 cm.

In some cases, the nanoelectroporation of the extracellular vesicle donor cells can comprise a cycle comprising nanochannel electroporation (CNP) followed by collecting the extracellular vesicles produced and secreted by the nonelectroporated extracellular vesicle donor cells for 1 hour, 2 hours, 3 hours, 4 hours, 5 hours, 6 hours, 12 hours, 24 hours, 36 hours, 48 hours, 3 days, 4 days, 5 days, 6 days, 1 week, or longer period of time for collecting the extracellular vesicles. In some cases, the extracellular vesicle donor cells can produce and secrete extracellular vesicles for 1 cycle, 2 cycles, 3 cycles, 4 cycles, 5 cycles, 6 cycles, 7 cycles, 8 cycles, 9 cycles, 10 cycles, or more cycles of CNP.

In some cases, the secreted extracellular vesicles are biocompatible, measure 40-150 nm in diameter, and may intrinsically express transmembrane and membrane-anchored proteins. The presence of these proteins may prolong blood circulation, may promote tissue-directed delivery and may facilitate cellular uptake of encapsulated exosomal contents. In some cases, the methods provided herein may involve use of nanoelectroporation without formation of significant aggregates. In some instances, the heterologous polynucleotide or vector introduced into the extracellular vesicle donor cell does not encode a peptide sequence that is incorporated into the extracellular vesicles and binds to target mRNA.

In some cases, the nanochannels comprise the pores of the semi-permeable polymer substrate. In some embodiment, the nanochannels comprise a height from about 0.01 μm to about 500 μm. In some embodiment, the nanochannels comprise a height from about 0.01 μm to about 0.05 μm, about 0.01 μm to about 0.1 μm, about 0.01 μm to about 0.5 μm, about 0.01 μm to about 1 μm, about 0.01 μm to about 2 μm, about 0.01 μm to about 5 μm, about 0.01 μm to about 10 μm, about 0.01 μm to about 20 μm, about 0.01 μm to about 50 μm, about 0.01 μm to about 100 μm, about 0.01 μm to about 500 μm, about 0.05 μm to about 0.1 μm, about 0.05 μm to about 0.5 μm, about 0.05 μm to about 1 μm, about 0.05 μm to about 2 μm, about 0.05 μm to about 5 μm, about 0.05 μm to about 10 μm, about 0.05 μm to about 20 μm, about 0.05 μm to about 50 μm, about 0.05 μm to about 100 μm, about 0.05 μm to about 500 μm, about 0.1 μm to about 0.5 μm, about 0.1 μm to about 1 μm, about 0.1 μm to about 2 μm, about 0.1 μm to about 5 μm, about 0.1 μm to about 10 μm, about 0.1 μm to about 20 μm, about 0.1 μm to about 50 μm, about 0.1 μm to about 100 μm, about 0.1 μm to about 500 μm, about 0.5 μm to about 1 μm, about 0.5 μm to about 2 μm, about 0.5 μm to about 5 μm, about 0.5 μm to about 10 μm, about 0.5 μm to about 20 μm, about 0.5 μm to about 50 μm, about 0.5 μm to about 100 μm, about 0.5 μm to about 500 μm, about 1 μm to about 2 μm, about 1 μm to about 5 μm, about 1 μm to about 10 μm, about 1 μm to about 20 μm, about 1 μm to about 50 μm, about 1 μm to about 100 μm, about 1 μm to about 500 μm, about 2 μm to about 5 μm, about 2 μm to about 10 μm, about 2 μm to about 20 μm, about 2 μm to about 50 μm, about 2 μm to about 100 μm, about 2 μm to about 500 μm, about 5 μm to about 10 μm, about 5 μm to about 20 μm, about 5 μm to about 50 μm, about 5 μm to about 100 μm, about 5 μm to about 500 μm, about 10 μm to about 20 μm, about 10 μm to about 50 μm, about 10 μm to about 100 μm, about 10 μm to about 500 μm, about 20 μm to about 50 μm, about 20 μm to about 100 μm, about 20 μm to about 500 μm, about 50 μm to about 100 μm, about 50 μm to about 500 μm, or about 100 μm to about 500 μm. In some embodiment, the nanochannels comprise a height from about 0.01 μm, about 0.05 μm, about 0.1 μm, about 0.5 μm, about 1 μm, about 2 μm, about 5 μm, about 10 μm, about 20 μm, about 50 μm, about 100 μm, or about 500 μm. In some embodiment, the nanochannels comprise a height from at least about 0.01 μm, about 0.05 μm, about 0.1 μm, about 0.5 μm, about 1 μm, about 2 μm, about 5 μm, about 10 μm, about 20 μm, about 50 μm, or about 100 μm. In some embodiment, the nanochannels comprise a height from at most about 0.05 μm, about 0.1 μm, about 0.5 μm, about 1 μm, about 2 μm, about 5 μm, about 10 μm, about 20 μm, about 50 μm, about 100 μm, or about 500 μm. In some cases, the heights of the nanochannels can be the same. In some cases, the heights of the nanochannels can be different. In some cases, the heights of the nanochannels should be great enough to accelerate the molecules being nanoelectroporated in the high electric field zone (e.g., inside the nanochannel), but also small enough to enable large molecules being nanoelectroporated to squeeze through in a brief electric pulse.

In some embodiment, the nanochannels comprise a diameter from about 0.01 nm to about 10,000 nm. In some embodiment, the nanochannels comprise a diameter from about 0.01 nm to about 0.1 nm, about 0.01 nm to about 0.5 nm, about 0.01 nm to about 1 nm, about 0.01 nm to about 5 nm, about 0.01 nm to about 10 nm, about 0.01 nm to about 50 nm, about 0.01 nm to about 100 nm, about 0.01 nm to about 500 nm, about 0.01 nm to about 1,000 nm, about 0.01 nm to about 5,000 nm, about 0.01 nm to about 10,000 nm, about 0.1 nm to about 0.5 nm, about 0.1 nm to about 1 nm, about 0.1 nm to about 5 nm, about 0.1 nm to about 10 nm, about 0.1 nm to about 50 nm, about 0.1 nm to about 100 nm, about 0.1 nm to about 500 nm, about 0.1 nm to about 1,000 nm, about 0.1 nm to about 5,000 nm, about 0.1 nm to about 10,000 nm, about 0.5 nm to about 1 nm, about 0.5 nm to about 5 nm, about 0.5 nm to about 10 nm, about 0.5 nm to about 50 nm, about 0.5 nm to about 100 nm, about 0.5 nm to about 500 nm, about 0.5 nm to about 1,000 nm, about 0.5 nm to about 5,000 nm, about 0.5 nm to about 10,000 nm, about 1 nm to about 5 nm, about 1 nm to about 10 nm, about 1 nm to about 50 nm, about 1 nm to about 100 nm, about 1 nm to about 500 nm, about 1 nm to about 1,000 nm, about 1 nm to about 5,000 nm, about 1 nm to about 10,000 nm, about 5 nm to about 10 nm, about 5 nm to about 50 nm, about 5 nm to about 100 nm, about 5 nm to about 500 nm, about 5 nm to about 1,000 nm, about 5 nm to about 5,000 nm, about 5 nm to about 10,000 nm, about 10 nm to about 50 nm, about 10 nm to about 100 nm, about 10 nm to about 500 nm, about 10 nm to about 1,000 nm, about 10 nm to about 5,000 nm, about 10 nm to about 10,000 nm, about 50 nm to about 100 nm, about 50 nm to about 500 nm, about 50 nm to about 1,000 nm, about 50 nm to about 5,000 nm, about 50 nm to about 10,000 nm, about 100 nm to about 500 nm, about 100 nm to about 1,000 nm, about 100 nm to about 5,000 nm, about 100 nm to about 10,000 nm, about 500 nm to about 1,000 nm, about 500 nm to about 5,000 nm, about 500 nm to about 10,000 nm, about 1,000 nm to about 5,000 nm, about 1,000 nm to about 10,000 nm, or about 5,000 nm to about 10,000 nm. In some embodiment, the nanochannels comprise a diameter from about 0.01 nm, about 0.1 nm, about 0.5 nm, about 1 nm, about 5 nm, about 10 nm, about 50 nm, about 100 nm, about 500 nm, about 1,000 nm, about 5,000 nm, or about 10,000 nm. In some embodiment, the nanochannels comprise a diameter from at least about 0.01 nm, about 0.1 nm, about 0.5 nm, about 1 nm, about 5 nm, about 10 nm, about 50 nm, about 100 nm, about 500 nm, about 1,000 nm, or about 5,000 nm. In some embodiment, the nanochannels comprise a diameter from at most about 0.1 nm, about 0.5 nm, about 1 nm, about 5 nm, about 10 nm, about 50 nm, about 100 nm, about 500 nm, about 1,000 nm, about 5,000 nm, or about 10,000 nm. In some cases, the diameters of the nanochannels can be the same. In some cases, the diameters of the nanochannels can be different.

In some cases, the nanochannels can be arranged into a nanochannel array. In some cases, the nanochannels can be arranged into a nanochannel array with spacing between the nanochannels. In some instances, the spacing between the nanochannels can be from about 0.01 μm to about 5,000 μm. In some instances, the spacing between the nanochannels can be from about 0.01 μm to about 0.05 μm, about 0.01 μm to about 0.1 μm, about 0.01 μm to about 0.5 μm, about 0.01 μm to about 1 μm, about 0.01 μm to about 5 μm, about 0.01 μm to about 10 μm, about 0.01 μm to about 50 μm, about 0.01 μm to about 100 μm, about 0.01 μm to about 500 μm, about 0.01 μm to about 1,000 μm, about 0.01 μm to about 5,000 μm, about 0.05 μm to about 0.1 μm, about 0.05 μm to about 0.5 μm, about 0.05 μm to about 1 μm, about 0.05 μm to about 5 μm, about 0.05 μm to about 10 μm, about 0.05 μm to about 50 μm, about 0.05 μm to about 100 μm, about 0.05 μm to about 500 μm, about 0.05 μm to about 1,000 μm, about 0.05 μm to about 5,000 μm, about 0.1 μm to about 0.5 μm, about 0.1 μm to about 1 μm, about 0.1 μm to about 5 μm, about 0.1 μm to about 10 μm, about 0.1 μm to about 50 μm, about 0.1 μm to about 100 μm, about 0.1 μm to about 500 μm, about 0.1 μm to about 1,000 μm, about 0.1 μm to about 5,000 μm, about 0.5 μm to about 1 μm, about 0.5 μm to about 5 μm, about 0.5 μm to about 10 μm, about 0.5 μm to about 50 μm, about 0.5 μm to about 100 μm, about 0.5 μm to about 500 μm, about 0.5 μm to about 1,000 μm, about 0.5 μm to about 5,000 μm, about 1 μm to about 5 μm, about 1 μm to about 10 μm, about 1 μm to about 50 μm, about 1 μm to about 100 μm, about 1 μm to about 500 μm, about 1 μm to about 1,000 μm, about 1 μm to about 5,000 μm, about 5 μm to about 10 μm, about 5 μm to about 50 μm, about 5 μm to about 100 μm, about 5 μm to about 500 μm, about 5 μm to about 1,000 μm, about 5 μm to about 5,000 μm, about 10 μm to about 50 μm, about 10 μm to about 100 μm, about 10 μm to about 500 μm, about 10 μm to about 1,000 μm, about 10 μm to about 5,000 μm, about 50 μm to about 100 μm, about 50 μm to about 500 μm, about 50 μm to about 1,000 μm, about 50 μm to about 5,000 μm, about 100 μm to about 500 μm, about 100 μm to about 1,000 μm, about 100 μm to about 5,000 μm, about 500 μm to about 1,000 μm, about 500 μm to about 5,000 μm, or about 1,000 μm to about 5,000 μm. In some instances, the spacing between the nanochannels can be from about 0.01 μm, about 0.05 μm, about 0.1 μm, about 0.5 μm, about 1 μm, about 5 μm, about 10 μm, about 50 μm, about 100 μm, about 500 μm, about 1,000 μm, or about 5,000 μm. In some instances, the spacing between the nanochannels can be from at least about 0.01 μm, about 0.05 μm, about 0.1 μm, about 0.5 μm, about 1 μm, about 5 μm, about 10 μm, about 50 μm, about 100 μm, about 500 μm, or about 1,000 μm. In some instances, the spacing between the nanochannels can be from at most about 0.05 μm, about 0.1 μm, about 0.5 μm, about 1 μm, about 5 μm, about 10 μm, about 50 μm, about 100 μm, about 500 μm, about 1,000 μm, or about 5,000 μm.

In some cases, the nanoelectroporating systems comprise upper and lower electrode layers for generating an electric field within the fluidic chamber. In some cases, the electric field generated by the electrodes for nanoelectroporation comprises a voltage that is between about 10 V to about 500 V. In some cases, the electric field generated by the electrodes for nanoelectroporation comprises a voltage that is between about 10 V to about 25 V, about 10 V to about 50 V, about 10 V to about 100 V, about 10 V to about 125 V, about 10 V to about 150 V, about 10 V to about 175 V, about 10 V to about 200 V, about 10 V to about 225 V, about 10 V to about 250 V, about 10 V to about 300 V, about 10 V to about 500 V, about 25 V to about 50 V, about 25 V to about 100 V, about 25 V to about 125 V, about 25 V to about 150 V, about 25 V to about 175 V, about 25 V to about 200 V, about 25 V to about 225 V, about 25 V to about 250 V, about 25 V to about 300 V, about 25 V to about 500 V, about 50 V to about 100 V, about 50 V to about 125 V, about 50 V to about 150 V, about 50 V to about 175 V, about 50 V to about 200 V, about 50 V to about 225 V, about 50 V to about 250 V, about 50 V to about 300 V, about 50 V to about 500 V, about 100 V to about 125 V, about 100 V to about 150 V, about 100 V to about 175 V, about 100 V to about 200 V, about 100 V to about 225 V, about 100 V to about 250 V, about 100 V to about 300 V, about 100 V to about 500 V, about 125 V to about 150 V, about 125 V to about 175 V, about 125 V to about 200 V, about 125 V to about 225 V, about 125 V to about 250 V, about 125 V to about 300 V, about 125 V to about 500 V, about 150 V to about 175 V, about 150 V to about 200 V, about 150 V to about 225 V, about 150 V to about 250 V, about 150 V to about 300 V, about 150 V to about 500 V, about 175 V to about 200 V, about 175 V to about 225 V, about 175 V to about 250 V, about 175 V to about 300 V, about 175 V to about 500 V, about 200 V to about 225 V, about 200 V to about 250 V, about 200 V to about 300 V, about 200 V to about 500 V, about 225 V to about 250 V, about 225 V to about 300 V, about 225 V to about 500 V, about 250 V to about 300 V, about 250 V to about 500 V, or about 300 V to about 500 V. In some cases, the electric field generated by the electrodes for nanoelectroporation comprises a voltage that is between about 10 V, about 25 V, about 50 V, about 100 V, about 125 V, about 150 V, about 175 V, about 200 V, about 225 V, about 250 V, about 300 V, or about 500 V. In some cases, the electric field generated by the electrodes for nanoelectroporation comprises a voltage that is between at least about 10 V, about 25 V, about 50 V, about 100 V, about 125 V, about 150 V, about 175 V, about 200 V, about 225 V, about 250 V, or about 300 V. In some cases, the electric field generated by the electrodes for nanoelectroporation comprises a voltage that is between at most about 25 V, about 50 V, about 100 V, about 125 V, about 150 V, about 175 V, about 200 V, about 225 V, about 250 V, about 300 V, or about 500 V.

In some cases, the electric field generated by the electrodes for nanoelectroporation comprises an electric field strength from about 0.1 volt/mm to about 50,000 volt/mm In some cases, the electric field generated by the electrodes for nanoelectroporation comprises an electric field strength from about 0.1 volt/mm to about 0.5 volt/mm, about 0.1 volt/mm to about 1 volt/mm, about 0.1 volt/mm to about 5 volt/mm, about 0.1 volt/mm to about 10 volt/mm, about 0.1 volt/mm to about 50 volt/mm, about 0.1 volt/mm to about 100 volt/mm, about 0.1 volt/mm to about 500 volt/mm, about 0.1 volt/mm to about 1,000 volt/mm, about 0.1 volt/mm to about 5,000 volt/mm, about 0.1 volt/mm to about 10,000 volt/mm, about 0.1 volt/mm to about 50,000 volt/mm, about 0.5 volt/mm to about 1 volt/mm, about 0.5 volt/mm to about 5 volt/mm, about 0.5 volt/mm to about 10 volt/mm, about 0.5 volt/mm to about 50 volt/mm, about 0.5 volt/mm to about 100 volt/mm, about 0.5 volt/mm to about 500 volt/mm, about 0.5 volt/mm to about 1,000 volt/mm, about 0.5 volt/mm to about 5,000 volt/mm, about 0.5 volt/mm to about 10,000 volt/mm, about 0.5 volt/mm to about 50,000 volt/mm, about 1 volt/mm to about 5 volt/mm, about 1 volt/mm to about 10 volt/mm, about 1 volt/mm to about 50 volt/mm, about 1 volt/mm to about 100 volt/mm, about 1 volt/mm to about 500 volt/mm, about 1 volt/mm to about 1,000 volt/mm, about 1 volt/mm to about 5,000 volt/mm, about 1 volt/mm to about 10,000 volt/mm, about 1 volt/mm to about 50,000 volt/mm, about 5 volt/mm to about 10 volt/mm, about 5 volt/mm to about 50 volt/mm, about 5 volt/mm to about 100 volt/mm, about 5 volt/mm to about 500 volt/mm, about 5 volt/mm to about 1,000 volt/mm, about 5 volt/mm to about 5,000 volt/mm, about 5 volt/mm to about 10,000 volt/mm, about 5 volt/mm to about 50,000 volt/mm, about 10 volt/mm to about 50 volt/mm, about 10 volt/mm to about 100 volt/mm, about 10 volt/mm to about 500 volt/mm, about 10 volt/mm to about 1,000 volt/mm, about 10 volt/mm to about 5,000 volt/mm, about 10 volt/mm to about 10,000 volt/mm, about 10 volt/mm to about 50,000 volt/mm, about 50 volt/mm to about 100 volt/mm, about 50 volt/mm to about 500 volt/mm, about 50 volt/mm to about 1,000 volt/mm, about 50 volt/mm to about 5,000 volt/mm, about 50 volt/mm to about 10,000 volt/mm, about 50 volt/mm to about 50,000 volt/mm, about 100 volt/mm to about 500 volt/mm, about 100 volt/mm to about 1,000 volt/mm, about 100 volt/mm to about 5,000 volt/mm, about 100 volt/mm to about 10,000 volt/mm, about 100 volt/mm to about 50,000 volt/mm, about 500 volt/mm to about 1,000 volt/mm, about 500 volt/mm to about 5,000 volt/mm, about 500 volt/mm to about 10,000 volt/mm, about 500 volt/mm to about 50,000 volt/mm, about 1,000 volt/mm to about 5,000 volt/mm, about 1,000 volt/mm to about 10,000 volt/mm, about 1,000 volt/mm to about 50,000 volt/mm, about 5,000 volt/mm to about 10,000 volt/mm, about 5,000 volt/mm to about 50,000 volt/mm, or about 10,000 volt/mm to about 50,000 volt/mm In some cases, the electric field generated by the electrodes for nanoelectroporation comprises an electric field strength from about 0.1 volt/mm, about 0.5 volt/mm, about 1 volt/mm, about 5 volt/mm, about 10 volt/mm, about 50 volt/mm, about 100 volt/mm, about 500 volt/mm, about 1,000 volt/mm, about 5,000 volt/mm, about 10,000 volt/mm, or about 50,000 volt/mm In some cases, the electric field generated by the electrodes for nanoelectroporation comprises an electric field strength from at least about 0.1 volt/mm, about 0.5 volt/mm, about 1 volt/mm, about 5 volt/mm, about 10 volt/mm, about 50 volt/mm, about 100 volt/mm, about 500 volt/mm, about 1,000 volt/mm, about 5,000 volt/mm, or about 10,000 volt/mm In some cases, the electric field generated by the electrodes for nanoelectroporation comprises an electric field strength from at most about 0.5 volt/mm, about 1 volt/mm, about 5 volt/mm, about 10 volt/mm, about 50 volt/mm, about 100 volt/mm, about 500 volt/mm, about 1,000 volt/mm, about 5,000 volt/mm, about 10,000 volt/mm, or about 50,000 volt/mm.

In some instances, the electric field generated by the electrodes for nanoelectroporation comprises a plurality of pulses with pulse duration from about 0.01 millisecond/pulse to about 5,000 millisecond/pulse. In some instances, the electric field generated by the electrodes for nanoelectroporation comprises a plurality of pulses with pulse duration from about 0.01 millisecond/pulse to about 0.05 millisecond/pulse, about 0.01 millisecond/pulse to about 0.1 millisecond/pulse, about 0.01 millisecond/pulse to about 0.5 millisecond/pulse, about 0.01 millisecond/pulse to about 1 millisecond/pulse, about 0.01 millisecond/pulse to about 5 millisecond/pulse, about 0.01 millisecond/pulse to about 10 millisecond/pulse, about 0.01 millisecond/pulse to about 50 millisecond/pulse, about 0.01 millisecond/pulse to about 100 millisecond/pulse, about 0.01 millisecond/pulse to about 500 millisecond/pulse, about 0.01 millisecond/pulse to about 1,000 millisecond/pulse, about 0.01 millisecond/pulse to about 5,000 millisecond/pulse, about 0.05 millisecond/pulse to about 0.1 millisecond/pulse, about 0.05 millisecond/pulse to about 0.5 millisecond/pulse, about 0.05 millisecond/pulse to about 1 millisecond/pulse, about 0.05 millisecond/pulse to about 5 millisecond/pulse, about 0.05 millisecond/pulse to about 10 millisecond/pulse, about 0.05 millisecond/pulse to about 50 millisecond/pulse, about 0.05 millisecond/pulse to about 100 millisecond/pulse, about 0.05 millisecond/pulse to about 500 millisecond/pulse, about 0.05 millisecond/pulse to about 1,000 millisecond/pulse, about 0.05 millisecond/pulse to about 5,000 millisecond/pulse, about 0.1 millisecond/pulse to about 0.5 millisecond/pulse, about 0.1 millisecond/pulse to about 1 millisecond/pulse, about 0.1 millisecond/pulse to about 5 millisecond/pulse, about 0.1 millisecond/pulse to about 10 millisecond/pulse, about 0.1 millisecond/pulse to about 50 millisecond/pulse, about 0.1 millisecond/pulse to about 100 millisecond/pulse, about 0.1 millisecond/pulse to about 500 millisecond/pulse, about 0.1 millisecond/pulse to about 1,000 millisecond/pulse, about 0.1 millisecond/pulse to about 5,000 millisecond/pulse, about 0.5 millisecond/pulse to about 1 millisecond/pulse, about 0.5 millisecond/pulse to about 5 millisecond/pulse, about 0.5 millisecond/pulse to about 10 millisecond/pulse, about 0.5 millisecond/pulse to about 50 millisecond/pulse, about 0.5 millisecond/pulse to about 100 millisecond/pulse, about 0.5 millisecond/pulse to about 500 millisecond/pulse, about 0.5 millisecond/pulse to about 1,000 millisecond/pulse, about 0.5 millisecond/pulse to about 5,000 millisecond/pulse, about 1 millisecond/pulse to about 5 millisecond/pulse, about 1 millisecond/pulse to about 10 millisecond/pulse, about 1 millisecond/pulse to about 50 millisecond/pulse, about 1 millisecond/pulse to about 100 millisecond/pulse, about 1 millisecond/pulse to about 500 millisecond/pulse, about 1 millisecond/pulse to about 1,000 millisecond/pulse, about 1 millisecond/pulse to about 5,000 millisecond/pulse, about 5 millisecond/pulse to about 10 millisecond/pulse, about 5 millisecond/pulse to about 50 millisecond/pulse, about 5 millisecond/pulse to about 100 millisecond/pulse, about 5 millisecond/pulse to about 500 millisecond/pulse, about 5 millisecond/pulse to about 1,000 millisecond/pulse, about 5 millisecond/pulse to about 5,000 millisecond/pulse, about 10 millisecond/pulse to about 50 millisecond/pulse, about 10 millisecond/pulse to about 100 millisecond/pulse, about 10 millisecond/pulse to about 500 millisecond/pulse, about 10 millisecond/pulse to about 1,000 millisecond/pulse, about 10 millisecond/pulse to about 5,000 millisecond/pulse, about 50 millisecond/pulse to about 100 millisecond/pulse, about 50 millisecond/pulse to about 500 millisecond/pulse, about 50 millisecond/pulse to about 1,000 millisecond/pulse, about 50 millisecond/pulse to about 5,000 millisecond/pulse, about 100 millisecond/pulse to about 500 millisecond/pulse, about 100 millisecond/pulse to about 1,000 millisecond/pulse, about 100 millisecond/pulse to about 5,000 millisecond/pulse, about 500 millisecond/pulse to about 1,000 millisecond/pulse, about 500 millisecond/pulse to about 5,000 millisecond/pulse, or about 1,000 millisecond/pulse to about 5,000 millisecond/pulse. In some instances, the electric field generated by the electrodes for nanoelectroporation comprises a plurality of pulses with pulse duration from about 0.01 millisecond/pulse, about 0.05 millisecond/pulse, about 0.1 millisecond/pulse, about 0.5 millisecond/pulse, about 1 millisecond/pulse, about 5 millisecond/pulse, about 10 millisecond/pulse, about 50 millisecond/pulse, about 100 millisecond/pulse, about 500 millisecond/pulse, about 1,000 millisecond/pulse, or about 5,000 millisecond/pulse. In some instances, the electric field generated by the electrodes for nanoelectroporation comprises a plurality of pulses with pulse duration from at least about 0.01 millisecond/pulse, about 0.05 millisecond/pulse, about 0.1 millisecond/pulse, about 0.5 millisecond/pulse, about 1 millisecond/pulse, about 5 millisecond/pulse, about 10 millisecond/pulse, about 50 millisecond/pulse, about 100 millisecond/pulse, about 500 millisecond/pulse, or about 1,000 millisecond/pulse. In some instances, the electric field generated by the electrodes for nanoelectroporation comprises a plurality of pulses with pulse duration from at most about 0.05 millisecond/pulse, about 0.1 millisecond/pulse, about 0.5 millisecond/pulse, about 1 millisecond/pulse, about 5 millisecond/pulse, about 10 millisecond/pulse, about 50 millisecond/pulse, about 100 millisecond/pulse, about 500 millisecond/pulse, about 1,000 millisecond/pulse, or about 5,000 millisecond/pulse. In some cases, the nanoelectroporation comprises 1 pulse, 2 pulses, 3 pulses, 4 pulses, 5 pulses, 6 pulses, 7 pulses, 8 pulses, 9 pulses, 10 pulses, 11 pulses, 12 pulses, 13 pulses, 14 pulses, 15 pulses, 16 pulses, 17 pulses, 18 pulses, 19 pulses, 20 pulses or more.

In some cases, the extracellular vesicles produced and secreted by the extracellular vesicle donor cells are collected and purified from a cell culture medium by centrifugation or ultracentrifugation, which may allow the extracellular vesicles to be purified from other cellular debris or molecules based on the density of the extracellular vesicles.

In some cases, the methods and systems of producing the extracellular vesicles comprising the targeting polypeptides and/or the therapeutic polynucleotides comprise loading the nanochannels with the plurality of vectors to be nonelectroporated into the cells. In some cases, molecules other than vectors (e.g. proteins, biomolecules, compounds, etc) can be loaded into the nanochannels to be nanoelectroporated into the cells. In some cases, the electric field generated by the upper and the lower electrodes accelerate the vectors into the cells. In some cases, the electric field generated for nanoelectroporation creates pores in the cells of the membrane to allow the nanoelectroporation of the vectors. In some cases, the pores in the membrane of the extracellular vesicle donor cells can be formed at a focal point, e.g. exit of the nanochannel where the electric field directly contacts the cell membrane.

In some cases, a nanoelectroporated extracellular vesicle donor cell can produce and secrete at least 10%, 50%, 1 fold, 5 fold, 10 fold, 50 fold, 100 fold, 500 fold, 1000 fold, 5000 fold, or more extracellular vesicles than an extracellular vesicle donor cell transfected by non-nanoelectroporation (e.g. conventional bulk electroporation, gene gun, lipofectamine transfection, etc.) In some cases, an nanoelectroporated extracellular vesicle donor cell can produce and secrete a number of extracellular vesicles that is increased by at least 10%, 50%, 1 fold, 5 fold, 10 fold, 50 fold, 100 fold, 500 fold, 1000 fold, 5000 fold, or more compared to a number of extracellular vesicles produced and secreted by an extracellular vesicle donor cell stimulated by non-nanoelectroporation (e.g. conventional bulk electroporation, gene gun, lipofectamine transfection, global cellular stress response, starvation, hypoxia, and heat treatment, etc.)

In some cases, the extracellular vesicle donor cell can produce and secrete more extracellular vesicles, when the extracellular vesicle donor cell is cultured and nanoelectroporated at an increased temperature. For example, the extracellular vesicle donor cell is cultured and nanoelectroporated at 37° C. produces and secretes more extracellular vesicles than the extracellular vesicle donor cell cultured and nanoelectroporated at 4° C. In some cases, the extracellular vesicle donor cell produces and secretes at least 10%, 50%, 1 fold, 5 fold, 10 fold, 50 fold, 100 fold, 1000 fold, or more extracellular vesicles for each 1° C. increased over 4° C. during the culturing and nanoelectroporating of the extracellular vesicle donor cell.

In some cases, the extracellular vesicle donor cell can produce and secrete more extracellular vesicles, when the extracellular vesicle donor cell is cultured in a buffer comprising Ca²⁺. For example, after nanoelectroporation an extracellular vesicle donor cell cultured in a buffer comprising 500 nM Ca²⁺ produces and secretes more extracellular vesicles compared to if the extracellular vesicle donor cell is cultured in a buffer comprising no Ca²⁺ after nanoelectroporation. In some cases, the extracellular vesicle donor cell produces and secretes at least 10%, 50%, 1 fold, 5 fold, 10 fold, 50 fold, 100 fold, 1000 fold, or more extracellular vesicles when cultured in a buffer comprising increased concentration of Ca²⁺ after nanoelectroporation compared to if the extracellular vesicle donor cell is cultured in a buffer comprising no Ca2+ after nanoelectroporation. Example of the increased concentration of Ca²⁺ in the buffer includes 10 nM, 50 nM, 100 nM, 150 nM, 200 nM, 250 nM, 300 nM, 400 nM, 500 nM, 600 nM, 7000 nM, 800 nM, 900 nM, 1000 nM, 1100 nM, 1200 nM, 1300 nM, 1400 nM, 1500 nM, 2000 nM, 2500 nM, 3000 nM, 5000 nM, 10000 nM, or higher concentration of Ca²⁺.

In some cases, the extracellular vesicle donor cell can produce and secrete more extracellular vesicles, when the extracellular vesicle donor cell is transfected with the at least one heterologous polynucleotide comprising a vector encoding 6-kbp Achaete-Scute Complex Like-1 (Ascl1), 7-kbp Pou Domain Class 3 Transcription factor 2 (Pou3f2 or Brn2), and 9-kbp Myelin Transcription Factor 1 Like (Myt1l). By transfecting the extracellular vesicle donor cell with the 6-kbp Achaete-Scute Complex Like-1 (Ascl1), 7-kbp Pou Domain Class 3 Transcription factor 2 (Pou3f2 or Brn2), and 9-kbp Myelin Transcription Factor 1 Like (Myt1l), the number of extracellular vesicles produced and secreted by the extracellular vesicle donor cell stimulated by nanoelectroporation can be increased by at least 10%, 50%, 1 fold, 5 fold, 10 fold, 50 fold, 100 fold, 1000 fold, or more folds compared to the number of extracellular vesicles produced and secreted by nanoelectroporating the extracellular vesicle donor cell without being transfected with the 6-kbp Achaete-Scute Complex Like-1 (Ascl1), 7-kbp Pou Domain Class 3 Transcription factor 2 (Pou3f2 or Brn2), and 9-kbp Myelin Transcription Factor 1 Like (Myt1l).

In some instances, extracellular vesicles produced and secreted by nanoelectroporated extracellular vesicle donor cells comprise at least 50%, 1 fold, 2 fold, 5 fold, 100 fold, 500 fold, 1000 fold, or more therapeutic polynucleotides compared to extracellular vesicles produced and secreted by extracellular vesicle donor cells transfected by non-nanoelectroporation. In some cases, the therapeutic polynucleotides encapsulated by the extracellular vesicles produced and secreted by nanoelectroporated extracellular vesicle donor cells are at least 10%, 20%, 30%, 40%, 50%, 60%, 70%, 80%, 90%, 95%, 99%, or more likely to be intact for encoding therapeutic polypeptides than therapeutic polynucleotides encapsulated by the extracellular vesicles produced and secreted by extracellular vesicle donor cells transfected by non-nanoelectroporation.

In some cases, a microchannel-electroporated or nanochannel-electroporated extracellular vesicle donor cell produces and secretes an increased percentage of extracellular vesicles comprising at least one copy of the therapeutic polynucleotide compared to a percentage of extracellular vesicles comprising at least one copy of the therapeutic polynucleotide produced and secreted by an extracellular vesicle donor cell transfected by other methods of transfection (e.g. conventional bulk electroporation, gene gun, lipofectamine transfection, etc). In some cases, the percentage of extracellular vesicles comprising at least one copy of the therapeutic polynucleotide produced and secreted by microchannel electroporated or nanochannel electroporated extracellular vesicle donor cell is increased by at least 0.1 fold, 0.2 fold, 0.5 fold, 2 fold, 5 fold, 10 fold, 50 fold, 100 fold, 500 fold, 1,000 fold, 5,000 fold, 10,000 fold, or more compared to the percentage of extracellular vesicles comprising at least one copy of the therapeutic polynucleotide produced and secreted by extracellular vesicle donor cell transfected by other methods of transfection. In some cases, the percentage of extracellular vesicles comprising at least one copy of the therapeutic polynucleotide can be determined by measuring the number of extracellular vesicles comprising the at least one copy of the therapeutic polynucleotide produced and secreted by extracellular vesicle donor cells over a span of 1 minute, 10 minutes, 30 minutes, 1 hour, 2 hours, 3 hours, 4 hours, 5 hours, 6 hours, 12 hours, 24 hours, 36 hours, 48 hours, 72 hours, 4 days, 5 days, 6 days, 1 week, 2 weeks, 3 weeks, 1 month, 2 moths, or a longer span of time. In some cases, microchannel electroporated or nanochannel electroporated extracellular vesicle donor cell produces and secretes an increased number of extracellular vesicles comprising at least one copy of the therapeutic polynucleotide (e.g., therapeutic mRNA, therapeutic miRNA) compared to a number of extracellular vesicles comprising at least one copy of the therapeutic polynucleotide produced and secreted by extracellular vesicle donor cell transfected by other methods of transfection (e.g. conventional bulk electroporation, gene gun, lipofectamine transfection, etc). In some cases, the microchannel electroporated or nanochannel electroporated extracellular vesicle donor cell produces and secretes an increased number of extracellular vesicles comprising at least one copy of the therapeutic polynucleotide is increased by at least 0.1 fold, 0.2 fold, 0.5 fold, 2 fold, 5 fold, 10 fold, 50 fold, 100 fold, 500 fold, 1,000 fold, 5,000 fold, 10,000 fold, or more compared to extracellular vesicles produced and secreted by extracellular vesicle donor cell transfected by other methods of transfection (e.g. conventional bulk electroporation, gene gun, lipofectamine transfection, etc). In some cases, microchannel electroporated or nanochannel electroporated extracellular vesicle donor cell produces and secretes an increased number of extracellular vesicles, where at least 1 out of 500 extracellular vesicles, at least 1 out of 200 extracellular vesicles, at least 1 out of 100 extracellular vesicles, at least 1 out of 50 extracellular vesicles, at least 1 out of 25 extracellular vesicles, or at least 1 out of 10 extracellular vesicles comprise at least 1 copy of therapeutic polynucleotide (e.g., therapeutic mRNA, therapeutic miRNA).

### Pharmaceutical Compositions

In some cases, the extracellular vesicles can be formulated into pharmaceutical composition. In some cases, the pharmaceutical composition comprising the extracellular vesicles or exosomes can be administered to a subject by multiple administration routes, including but not limited to, parenteral, oral, buccal, rectal, sublingual, or transdermal administration routes. In some cases, parenteral administration comprises intravenous, subcutaneous, intramuscular, intracerebral, intranasal, intra-arterial, intra-articular, intradermal, intravitreal, intraosseous infusion, intraperitoneal, or intrathecal administration. In some instances, the pharmaceutical composition is formulated for local administration. In other instances, the pharmaceutical composition is formulated for systemic administration. In some cases, the pharmaceutical composition and formulations described herein are administered to a subject by intravenous, subcutaneous, and intramuscular administration. In some cases, the pharmaceutical composition and formulations described herein are administered to a subject by intravenous administration. In some cases, the pharmaceutical composition and formulations described herein are administered to a subject by administration. In some cases, the pharmaceutical composition and formulations described herein are administered to a subject by intramuscular administration.

### Kits/Article of Manufacture

Disclosed herein, in certain aspects, are kits and articles of manufacture for use with one or more methods and compositions described herein. Also described herein are systems of manufacturing the extracellular vesicles or exosomes. In some cases, the systems comprise methods to nanoelectroporate extracellular vesicle donor cells to stimulate the production of extracellular vesicles or exosomes comprising the targeting polypeptides and the therapeutic polynucleotides.

In some cases, the kits can include a carrier, package, or container that is compartmentalized to receive one or more containers such as vials, tubes, and the like, each of the container(s) comprising one of the separate elements to be used in the methods described herein. Suitable containers include, for example, bottles, vials, syringes, and test tubes. In some cases, the containers can be formed from a variety of materials such as glass or plastic. kit typically includes labels listing contents and/or instructions for use, and package inserts with instructions for use. A set of instructions will also typically be included.

In one embodiment, a label is on or associated with the container. In one embodiment, a label is on a container when letters, numbers or other characters forming the label are attached, molded or etched into the container itself, a label is associated with a container when it is present within a receptacle or carrier that also holds the container, e.g., as a package insert. In one embodiment, a label is used to indicate that the contents are to be used for a specific therapeutic application. The label also indicates directions for use of the contents, such as in the methods described herein.

In certain cases, the extracellular vesicles comprising the targeting polypeptides and the therapeutic polynucleotides can be presented in a pack or dispenser device which contains one or more unit dosage forms containing a compound provided herein. The pack, for example, contains metal or plastic foil, such as a blister pack. In one embodiment, the pack or dispenser device is accompanied by instructions for administration. In one embodiment, the pack or dispenser is also accompanied with a notice associated with the container in form prescribed by a governmental agency regulating the manufacture, use, or sale of pharmaceuticals, which notice is reflective of approval by the agency of the form of the drug for human or veterinary administration. Such notice, for example, is the labeling approved by the U.S. Food and Drug Administration for drugs, or the approved product insert. In one embodiment, the extracellular vesicles comprising the tumor targeting polypeptides and the therapeutic polynucleotides containing a compound provided herein formulated in a compatible pharmaceutical carrier are also prepared, placed in an appropriate container, and labeled for treatment of an indicated condition.

In some cases, the kits comprise articles of manufacture that are useful for developing adoptive therapies and methods of treatment described herein. In some cases, kits comprise at least one extracellular vesicle comprising the targeting polypeptides and the therapeutic polynucleotides or components to manufacture the at least one extracellular vesicles comprising the tumor targeting polypeptides and the therapeutic polynucleotides. In some cases, kits comprise at least one exosome comprising the targeting polypeptides and the therapeutic polynucleotides or components to manufacture the at least one exosome comprising the targeting polypeptides and the therapeutic polynucleotides.

### EXAMPLES

The following illustrative examples are representative of embodiments of the compositions, systems, and methods described herein and are not meant to be limiting in any way.

### Example 1. Quantification of Cellular Nanoporation (CNP) Generated Extracellular Vesicles

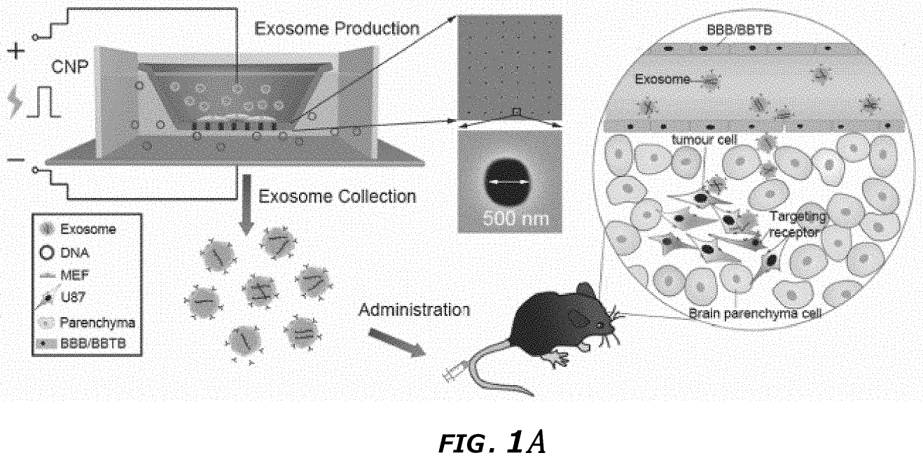

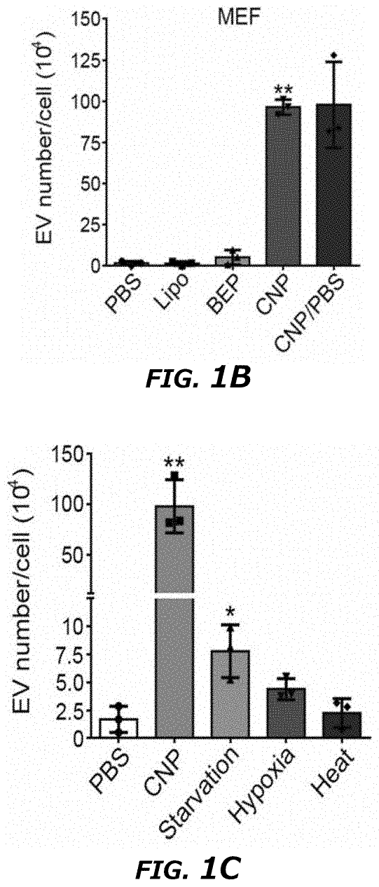

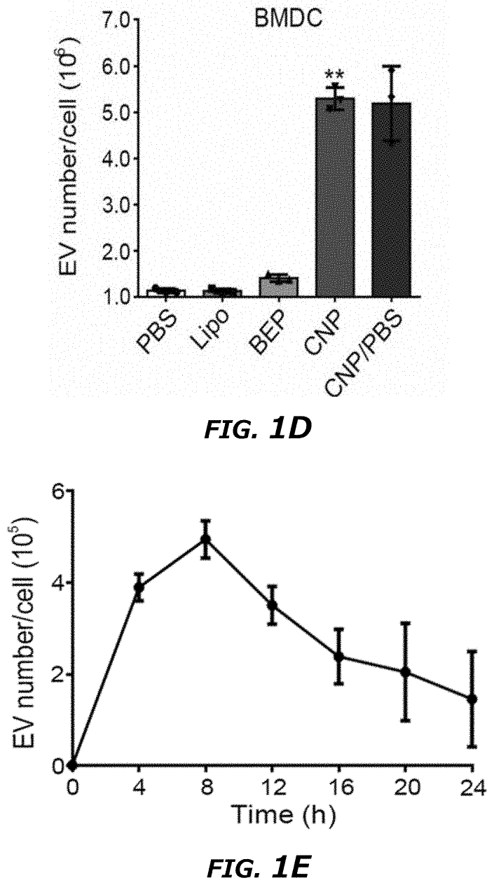

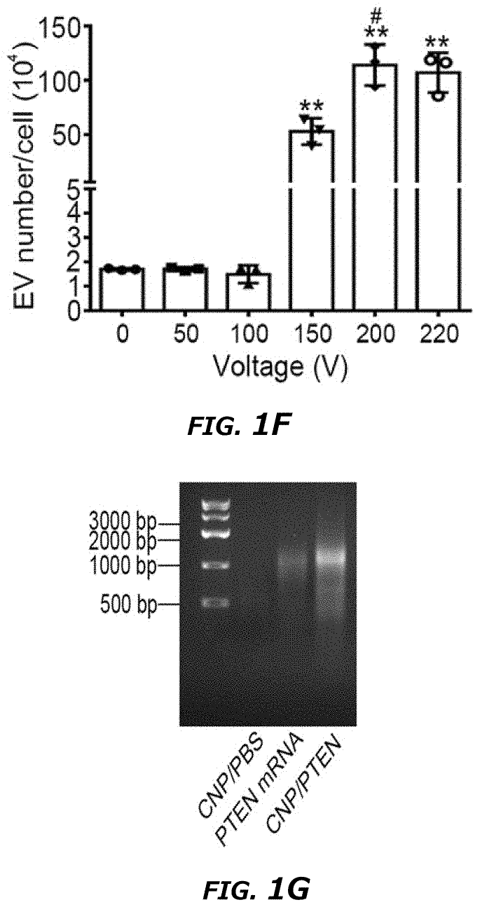

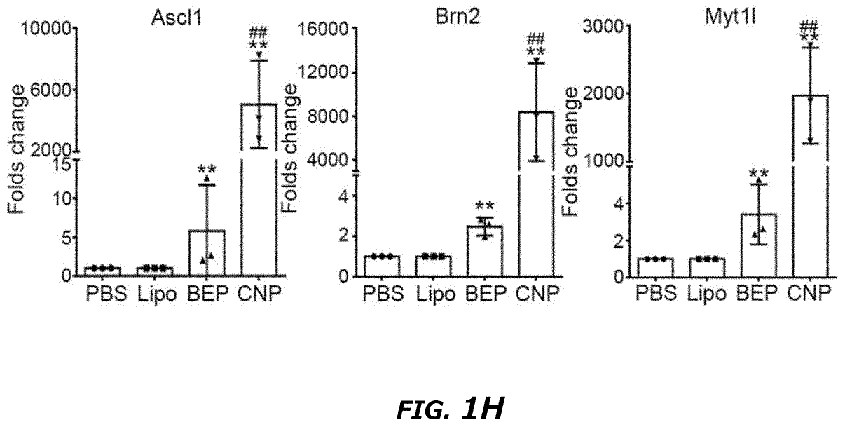

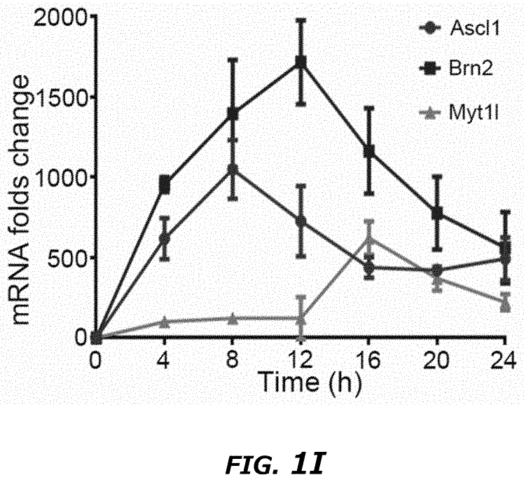

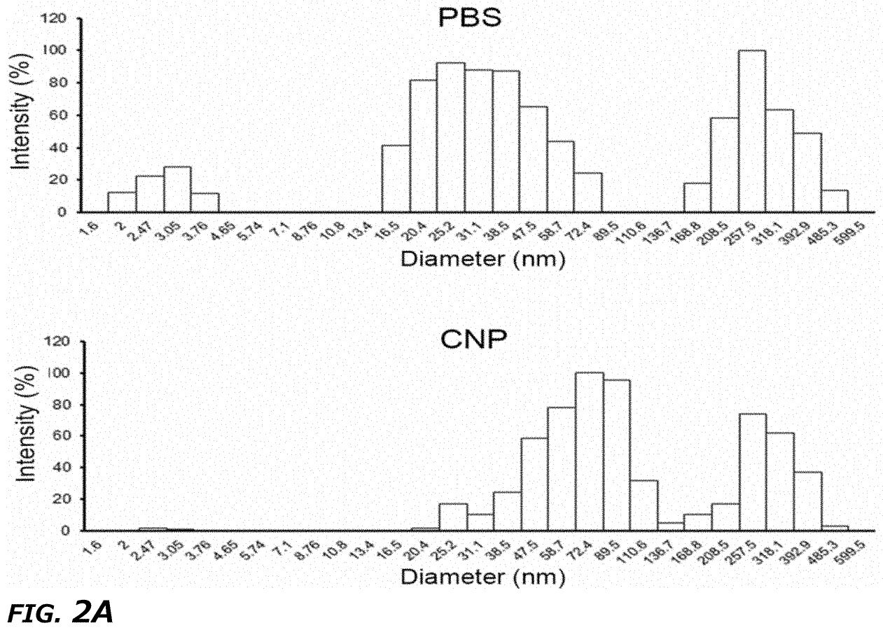

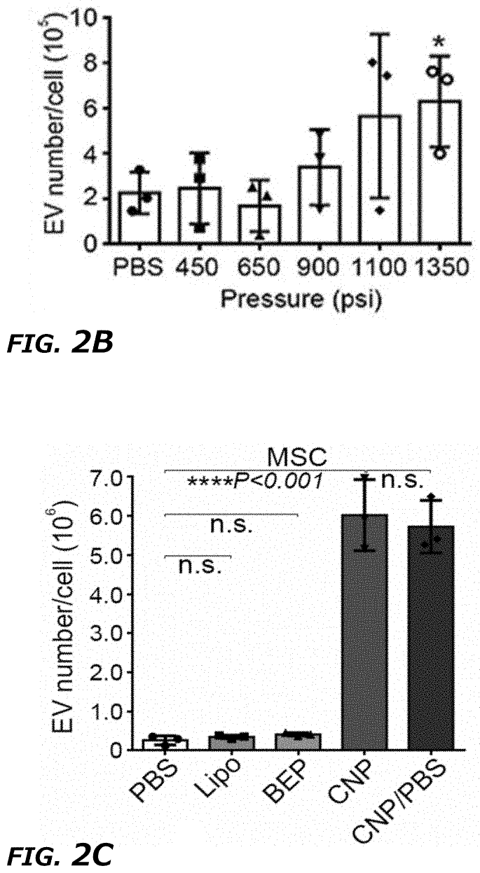

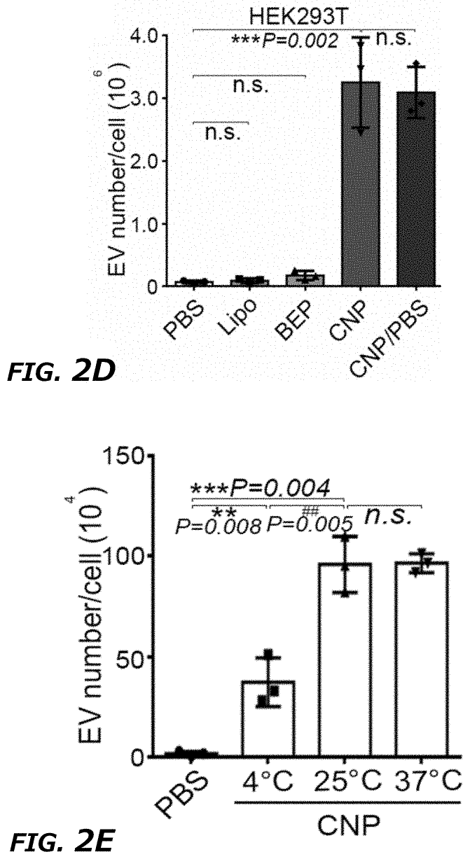

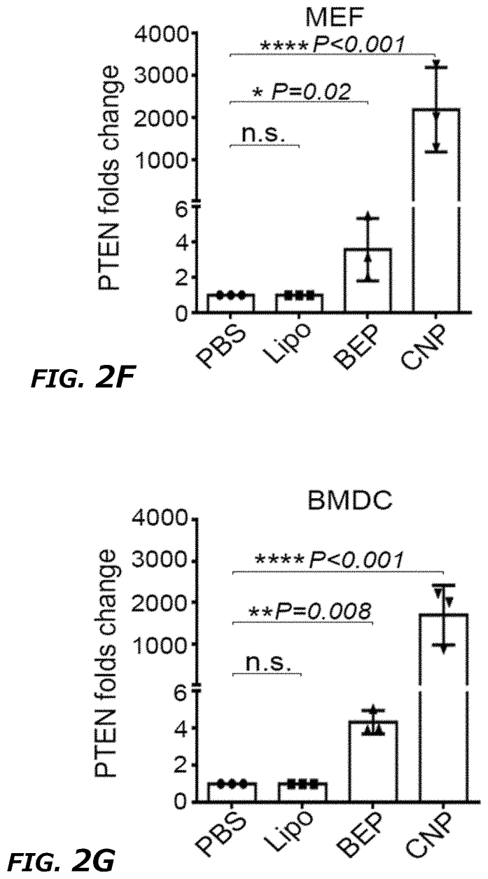

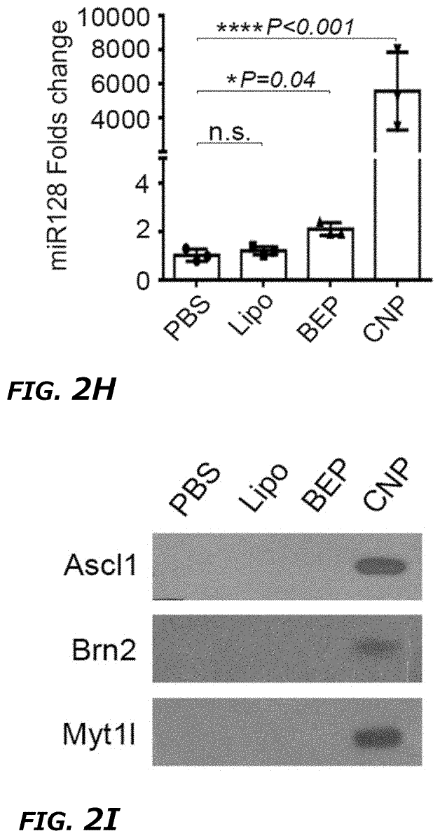

A cellular nanoporation (CNP) biochip, CNP system, and CNP method to stimulate cells to produce and release exosomes containing nucleotide sequences of interest including mRNA, microRNA and shRNA are developed and described herein. The system and method allowed a monolayer of source cells such as mouse embryonic fibroblasts (MEFs) and dendritic cells (DCs) to be cultured over the chip surface, which contained an array of nanochannels (FIG. 1A). The nanochannels (˜500 nm in diameter) enabled the passage of transient electrical pulses to shuttle DNA plasmids from buffer into the attached cells (FIG. 1A). Addition of 6-kbp Achaete-Scute Complex Like-1 (Ascl1, at times referred to herein as “A”), 7-kbp Pou Domain Class 3 Transcription factor 2 (Pou3f2 or Brn2, at times referred to herein as “B”) and 9-kbp Myelin Transcription Factor 1 Like (Myt1l, at times referred to herein as “M”) plasmids in buffer, resulted in a CNP yield of >50-fold increase in secreted extracellular vehicles (EVs) with similar vesicle size distribution as compared to other conventional techniques (FIG. 1B and FIG. 2A-B). Similarly, EV-production methods that relied on global cellular stress responses such as starvation, hypoxia, and heat treatment only resulted in moderate EV release (FIG. 1C). By comparison, the CNP-induced EV secretion was highly robust and could be applied to different cell sources types and transfection vectors (FIG. 1D and FIG. 2C-D). Kinetic analyses further showed that EV release peaked at 8 h after CNP-induction, with continued secretion noted over 24 h (FIG. 1E). The extent of EV secretion could be controlled by adjusting the voltage across the nanochannels, where a higher number of released EVs was observed when the voltage was increased from 100 to 150 V, until a plateau was reached at 200 V (FIG. 1F). Ambient temperature was another variable that could influence CNP triggered EV secretion, as cells prepared at 37° C. released more EVs as compared to 4° C. (FIG. 2E). To assess the internal nucleic acid content of released EVs, agarose gel analysis was performed with RNAs collected from EVs after source cells underwent CNP with PTEN plasmids. A higher number of intact PTEN mRNAs were packed within the EVs when compared to the CNP/PBS group (FIG. 1G), with a 55.5±9.2% by weight of total large RNA comprised of intact PTEN mRNA in CNP/PTEN plasmid-induced EVs. Quantitative reverse transcription polymerase chain reaction (qtr.-PCR) further confirmed that with CNP, a 103-fold increase was observed in mRNAs or miRNA complementary to the plasmid DNAs within the EVs relative to BEP or Lipo techniques (FIG. 1H and FIG. 2F-H). Additionally, the complementary mRNAs extracted from CNP generated EVs maintained their ability to encode polypeptides for protein synthesis (FIG. 2I). When multiple plasmid DNAs were used in CNP, the levels of complementary mRNAs exhibited a gradual increase with the largest transcript, Myt1l, taking the longest time (16 h) to reach the peak concentration (FIG. 1I), likely due to a longer time required for transcription of lengthy nucleic acid sequences.

### Example 2. Extracellular Vesicular mRNA Loading

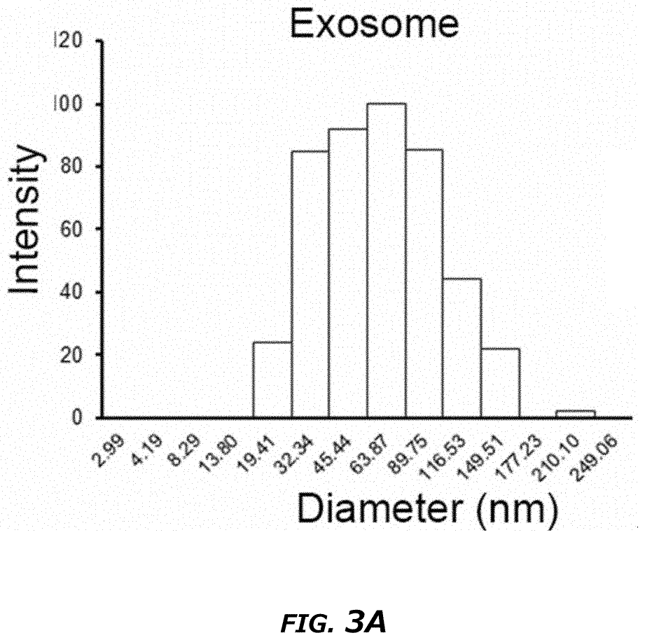

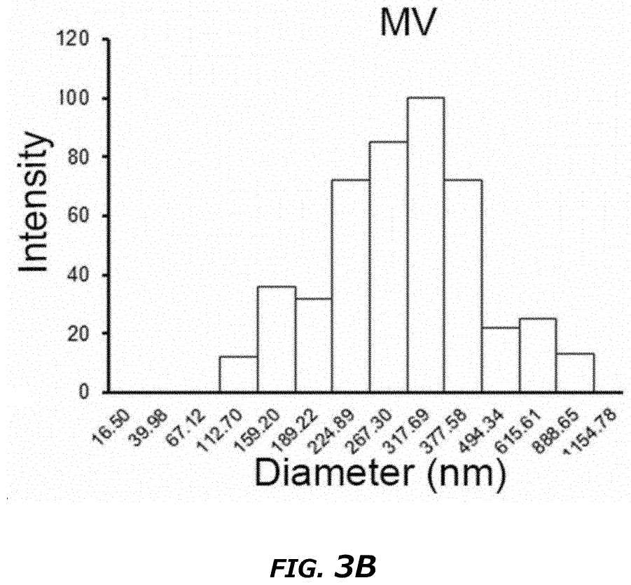

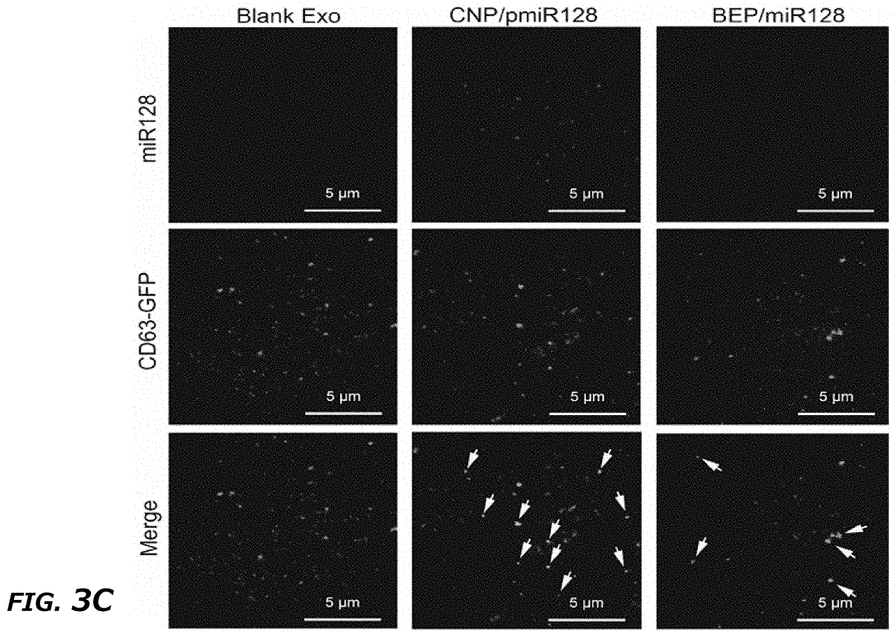

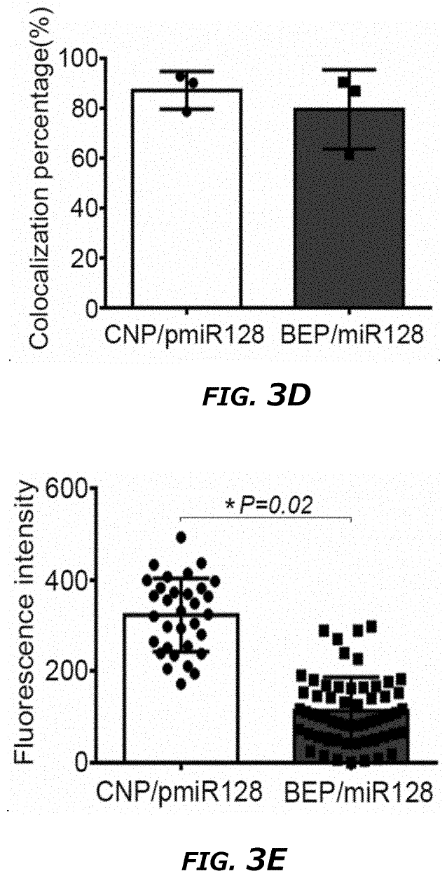

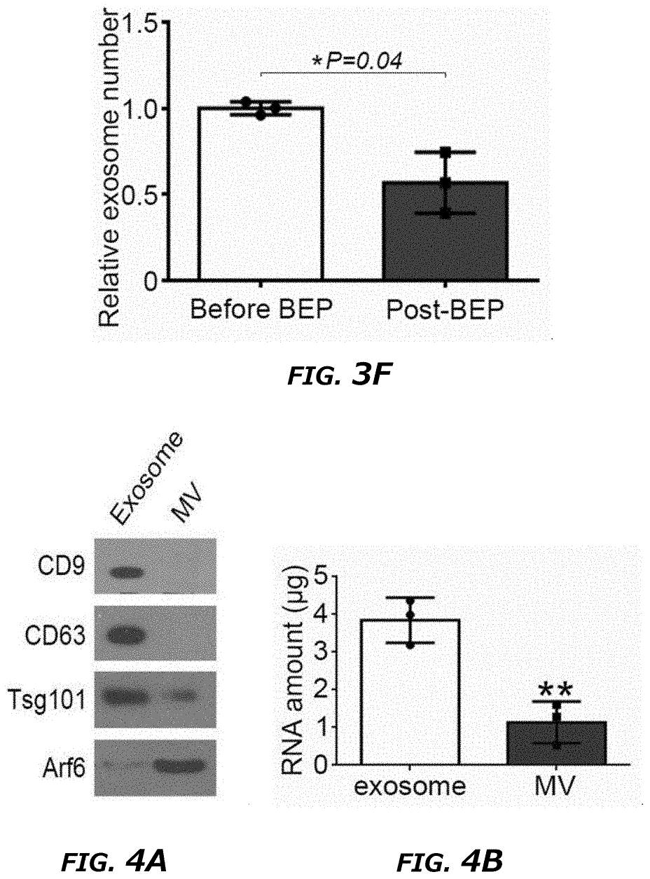

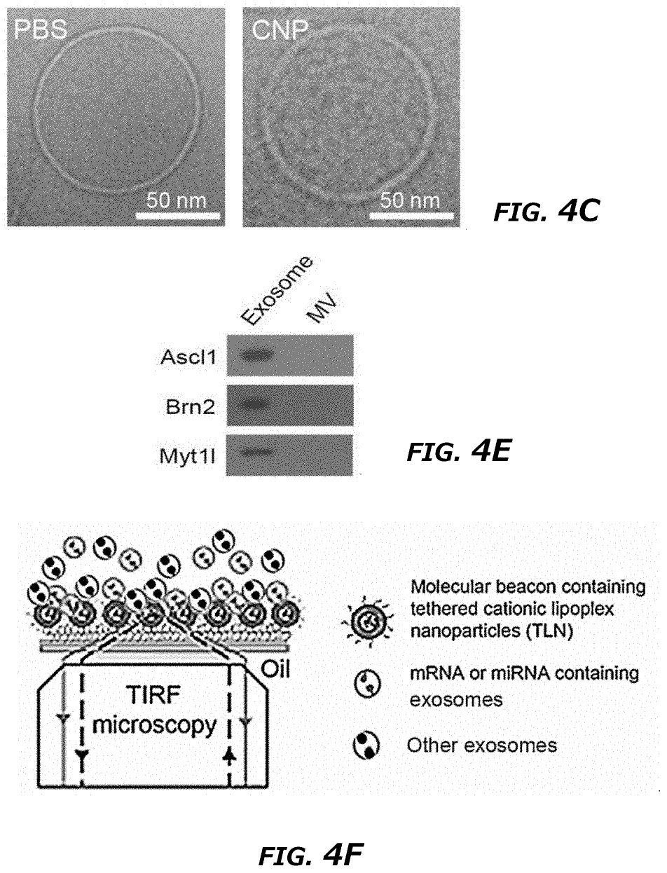

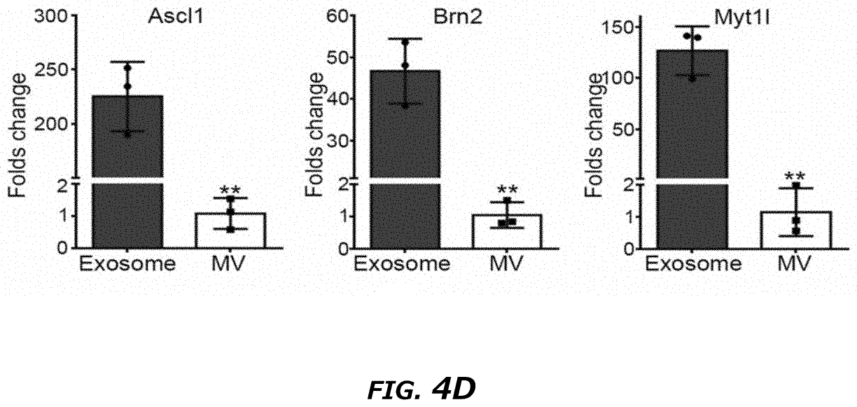

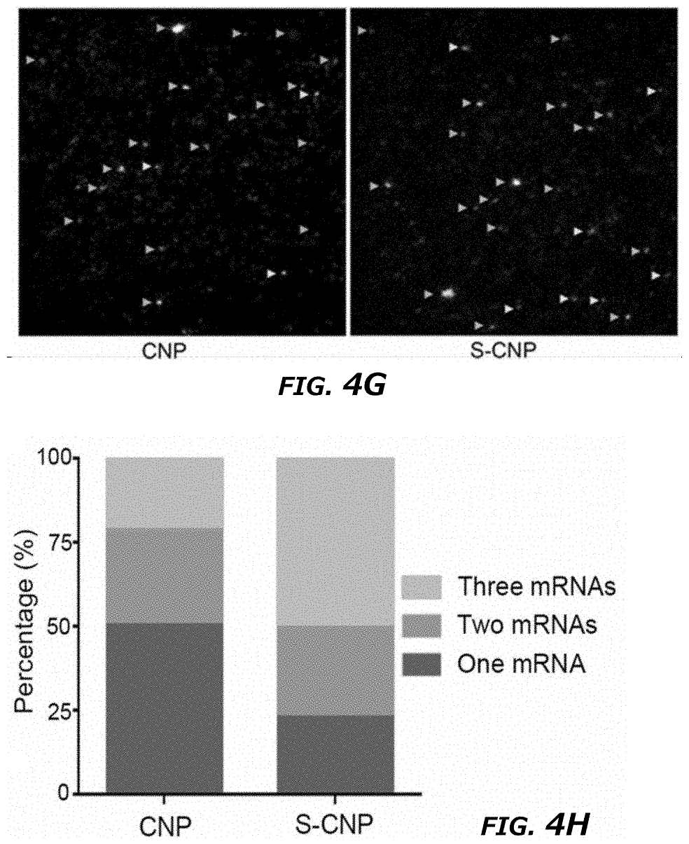

To better understand the distribution of the transcribed mRNA in different EV subgroups, exosomes were first separated from microvesicles (MV) by standard multi-step ultracentrifugation (FIG. 3A-B). Exosome markers (CD9, CD63, and Tsg101) and MV marker (Arf6) were detected by Western blot in exosomes and MVs (FIG. 4A). The majority (>75%) of the total EV RNA from 108 CNP transfected MEFs were within exosomes rather than in MVs (FIG. 4B), and the average weight ratio of large RNA/protein for exosomes was 1 μg/20 This was in contrast to non-detectable large RNA from MEF cells without CNP from the same number of exosomes. CryoTEM images in FIG. 4C further revealed that exosome generated by CNP with plasmid DNA contained many nucleic acids, while those from untreated MEFs were empty. qRT-PCR measurements further confirmed that majority of the transcribed mRNAs were also inside exosomes rather than in MVs (FIG. 4D), and that they maintained the ability to encode polypeptides for protein synthesis (FIG. 4E).

To further quantify the potential variability in terms of mRNA loading within individual exosomes, a tethered lipoplex nanoparticle (TLN) assay was utilized (FIG. 4F), where cationic lipoplex nanoparticles containing specific molecular beacon (MB) were tethered onto a glass coverslip, and the negatively charged exosomes were captured individually by nanoparticles using electrostatic interactions. The hybridization of mRNA inside the exosome with the MB inside the nanoparticles produced a fluorescence signal, which was captured by total internal refraction fluorescence (TIRF) microscopy to quantify the mRNA content. It was discovered that following CNP with 3 distinct DNA plasmids (A, B and M), approximately 50% of the exosomes contained only one transcript, 25% contained two mRNAs, and 25% of the exosomes included all three mRNA sequences (FIG. 4G-H). This multiplexed loading was improved by using a sequential CNP (S-CNP) technique (FIG. 4G-H), where different plasmids were delivered separately according to their peak-time (FIG. M. Therefore, S-CNP greatly improved multiple mRNA loadings into individual exosomes by more than 50%.

### Example 3. Comparison of CNP and BEP for Therapeutic Exosomes

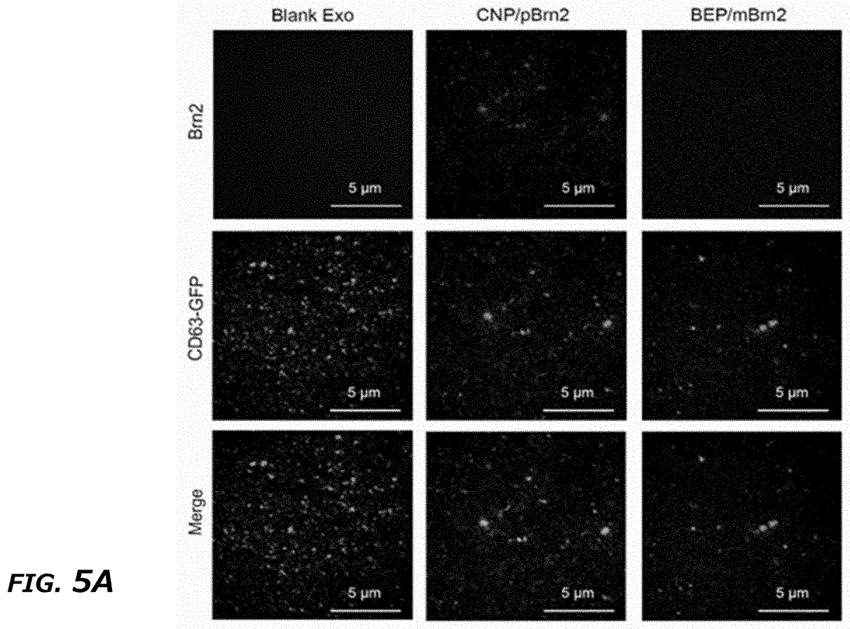

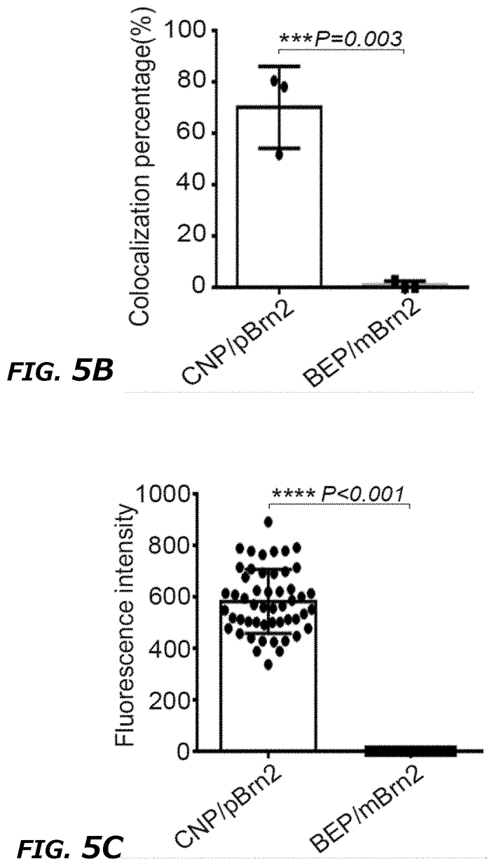

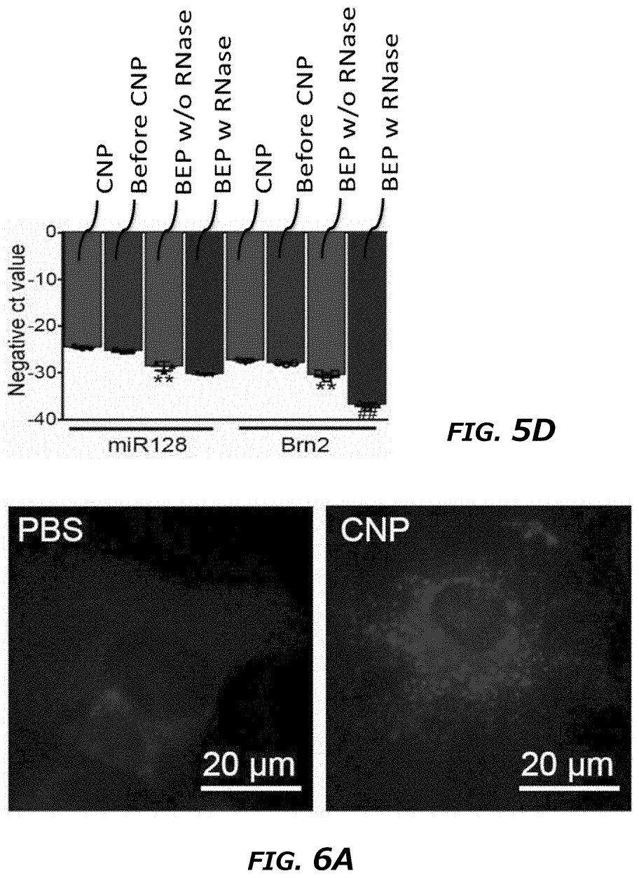

The central difference between CNP and existing BEP techniques is that CNP encapsulates endogenously transcribed RNAs into exosomes, whereas BEP delivers exogenous nucleotides into pre-isolated exosomes. To compare the efficiency between the two approaches, both miR-128 and CD63-GFP plasmids were first delivered into MEFs using CNP to generate GFP-labelled exosomes containing miR-128. Alternatively, free miR-128 was mixed with pre-isolated empty exosomes for BEP insertion. MicroRNA was used as nucleic acid cargo since BEP could only insert small nucleic acid sequences into exosomes. The capability of BEP to only encapsulate small nucleic acid sequences is a major limitation for its use in mRNA-based exosome generation. To compare the amount of miR-128 within both CNP and BEP prepared exosomes, a tethered lipoplex nanoparticle (TLN) biochip containing Cy5-miR128 molecular beacons was designed to capture negatively charged exosomes, enabling the fusion of the two vesicles. The subsequent hybridization of miR-128 molecules and Cy5-miR128 molecular beacons resulted in the emission of red fluorescence that can be captured by TIRF microscopy (FIG. 3C). Although both CNP and BEP produced ˜80% exosomes containing miR-128, the concentrations of miR-128 within CNP exosomes were much higher (FIG. 3D-F). Moreover, unlike BEP, CNP efficiently produced exosomes containing large mRNA (FIG. 5A-C), in which CNP-secreted exosomes contained >100 times more Brn2 mRNA than exosomes from BEP insertion (FIG. 1H and FIG. 5D).

### Example 4. Mechanisms of CNP-Induced Exosome Secretion

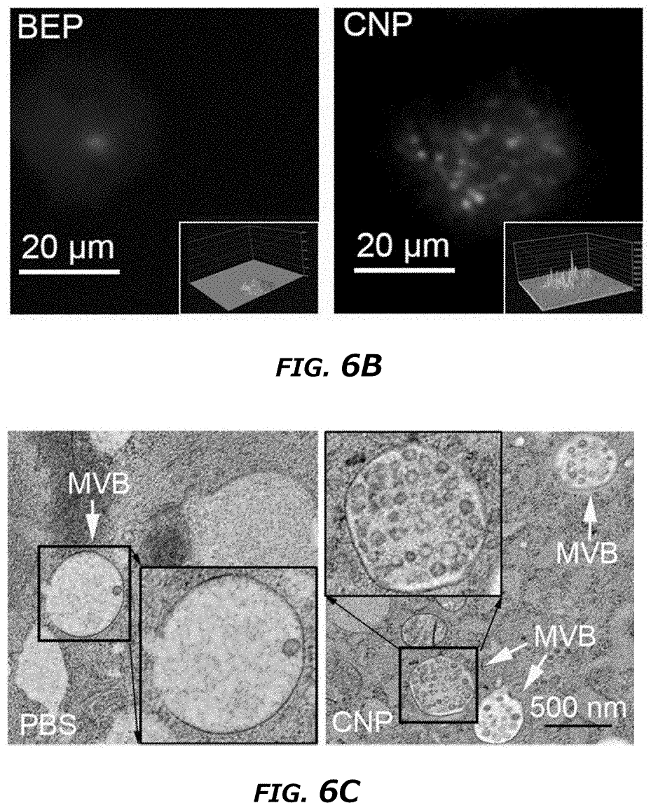

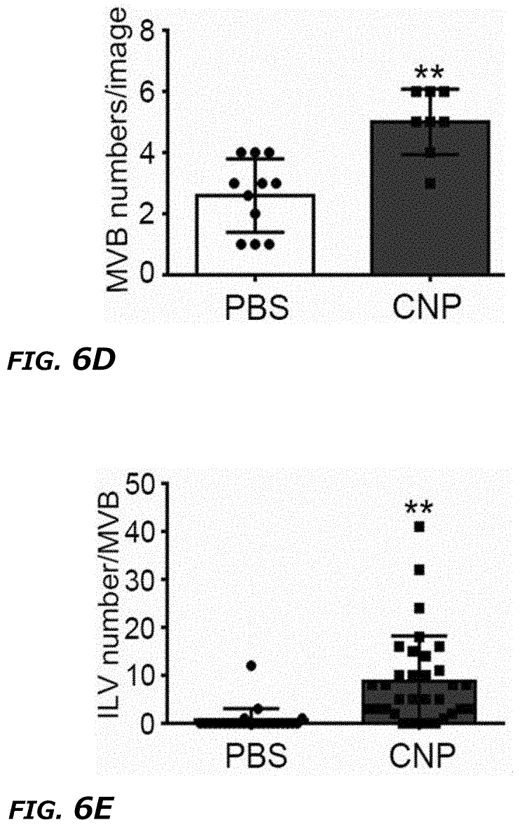

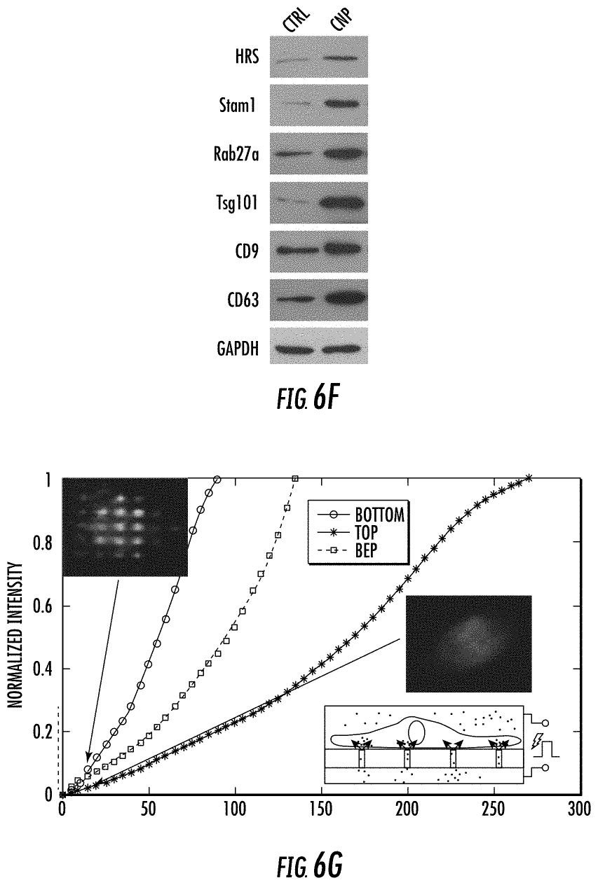

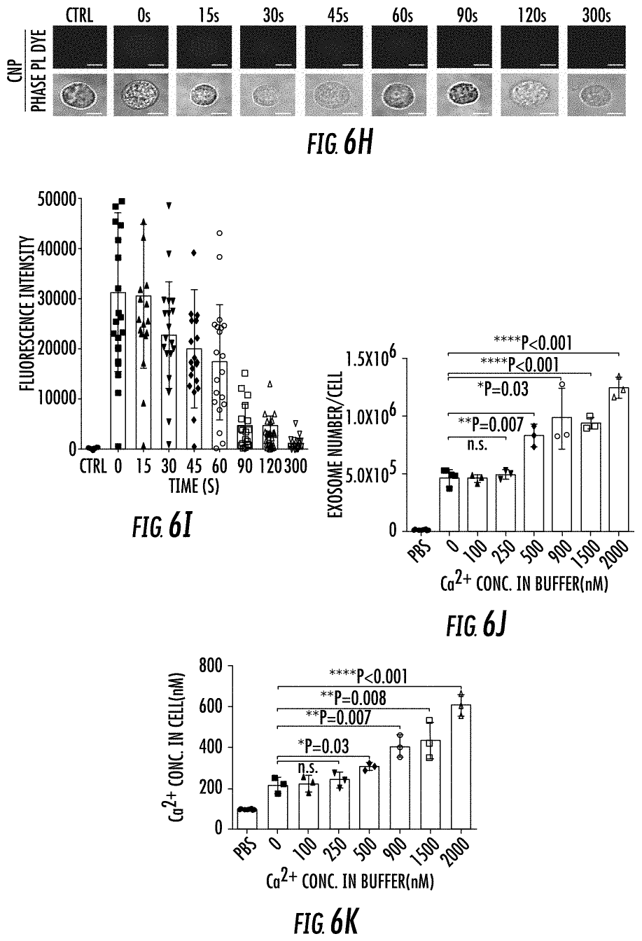

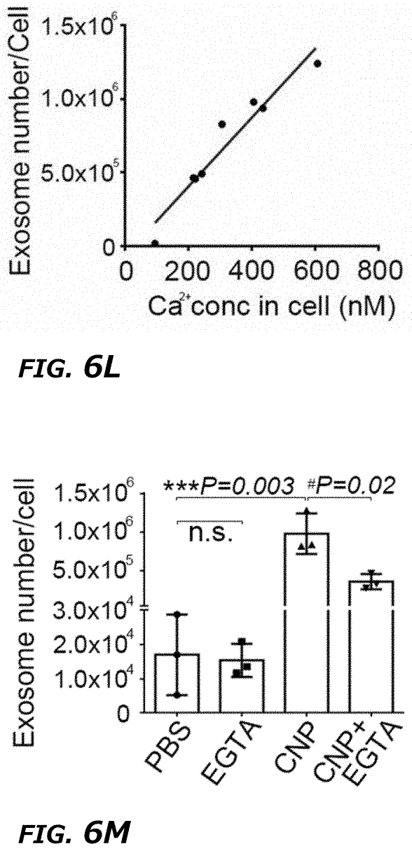

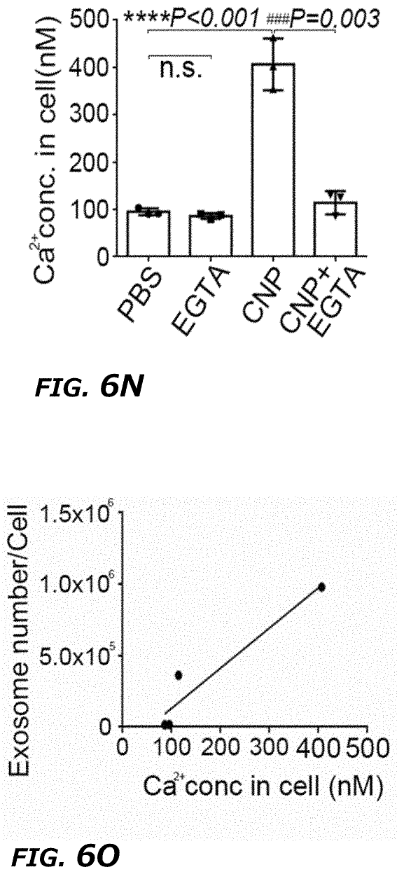

To investigate the cellular mechanisms underlying CNP-triggered exosome release, structural changes were first examined within the cell following CNP exposure. It was shown that CNP significantly increased the formation of multivesicular bodies (MVBs) within MEFs (FIG. 6A). When CD63-GFP plasmid was delivered to MEFs by CNP, a large number of GFP-positive MVBs were formed within 4 h after induction (FIG. 6B). Transmission electron microscopy (TEM) further revealed a ˜2 folds increase in MVBs and ˜8 times increases intraluminal vesicles (ILVs) in MEFs after CNP treatment (FIG. 6C-E). A corresponding increase in the expression of proteins involved in exosome biogenesis was observed (FIG. 6F). Since CNP relies on the delivery of focal electric fields across the source cell's plasma membrane, damages at the point of contact are likely to occur. It was shown that a large number of pores in the plasma membrane facing the basal surface were formed initially, followed by a gradual increase (slower than cells subjected to BEP) in fluorescence across the apical surface of the cell, suggesting that CNP caused small pores to form beyond the point of contact with the nanochannels (FIG. 6G). Interestingly, the cell membrane nanopores were found to close within 2 mins post electroporation, indicating that a recovery process likely occurred to repair the membrane damages (FIG. 6H-I). Since a higher intracellular calcium ion level has been reported to promote exosome release, the role played by influx of calcium ion through these nanopores after CNP resulted in higher levels of exosome secretion was examined. Indeed, it was found that increased exosome release after CNP was accomplished with increased Ca²⁺ in the extracellular space, with a corresponding increase in intracellular Ca²⁺ (FIG. 6J-L). Addition of a calcium chelator, EGTA, largely blocked the calcium ion rise inside the cells and inhibited the exosome release caused by CNP (FIG. 6M-N, suggesting that intracellular rise of Ca²⁺ was likely an initiating factor for the induction of exosome secretion by CNP (FIG. 6O).

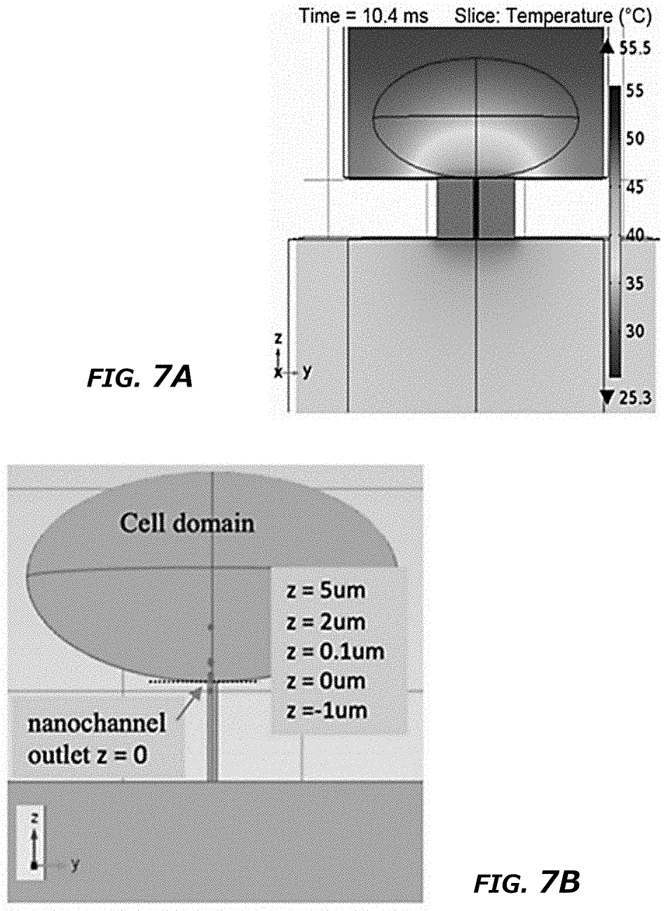

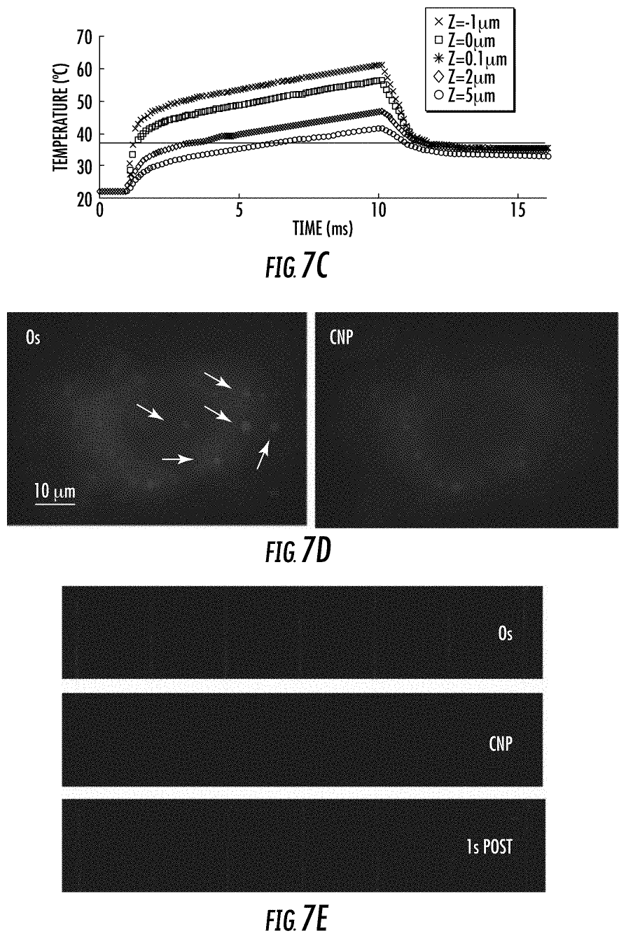

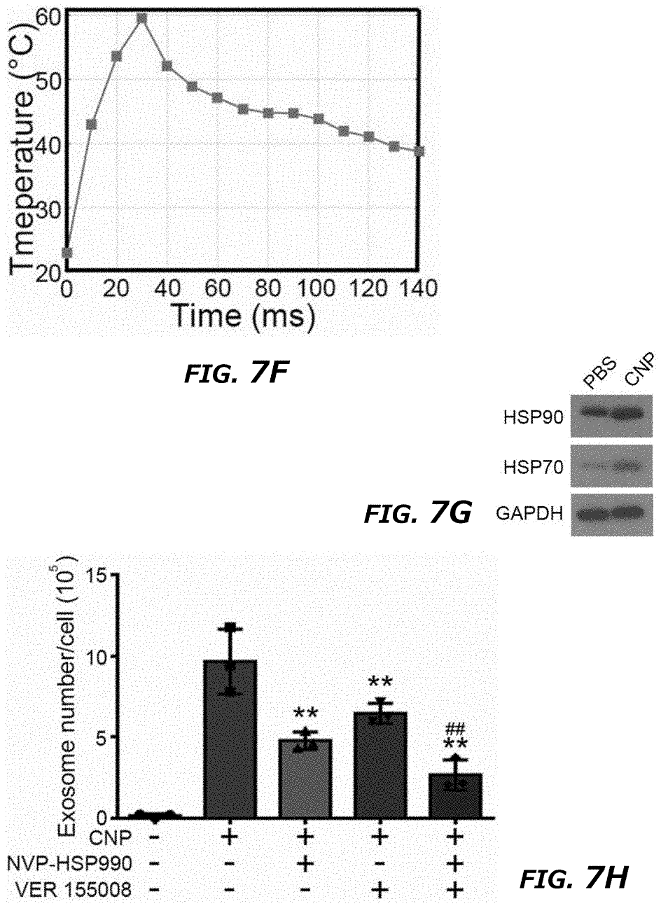

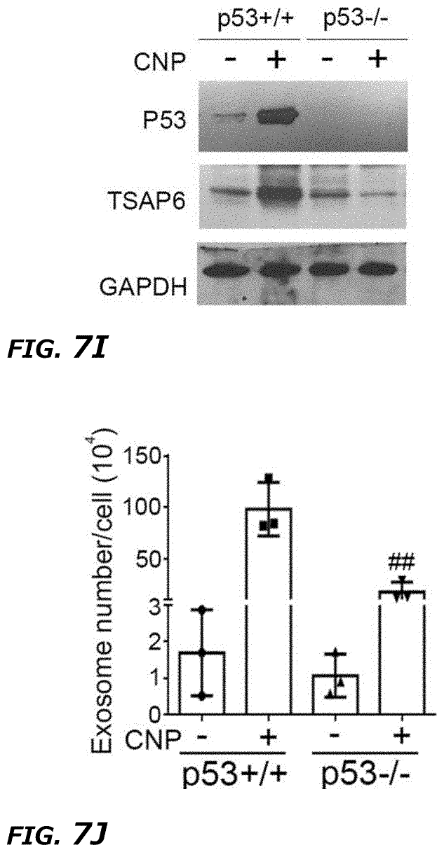

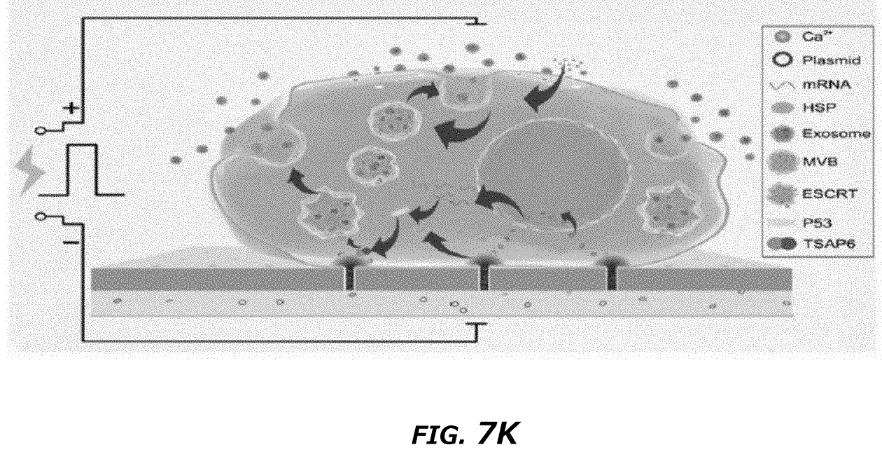

Next, stress responses within source cells contributed to the increased exosome formation following CNP treatment were assessed. Thermal shock, through increased production of heat-shock proteins (HSPs), has been shown to stimulate exosome biogenesis. Numerical simulation showed that transient (<1 s) increases in temperature approaching 60° C. around the nanochannel exit could occur during CNP (FIG. 7A-C). This temperature rise was focally oriented in the cell surface around the nanochannel exit (FIG. 7D-F). As expected, CNP substantially increased the expression of HSPs in cells, and the addition of HSP inhibitors was found to significantly suppress exosome secretion (FIG. 7G-H), suggesting that heat-shock response was critical for CNP-mediated exosome production. Since HSP could regulate P53 activity, which in turn regulated exosome production through TSAP6, whether elevation in HSP via CNP promoted exosome production through the P53-TSAP6 signaling pathway was examined. Accordingly, it was found that P53 and TSAP6 expression levels were upregulated following CNP (FIG. 7I), and in a P53 stable-knockdown MEF cell line (MEF P53−/−) TSAP6 expression was not changed (FIG. 7I). Furthermore, exosome release was not increased in P53−/− MEF cells following CNP (FIG. 7J). These results suggested that heat-shock responses in the setting of CNP-induced focal thermal stress promoted the activation of P53-TSAP6 signaling, leading to increased exosome production as a part of cellular recovery process (FIG. 7K).

### Example 5. Functional and Pharmacokinetic Evaluation of CNP Generated Exosomes

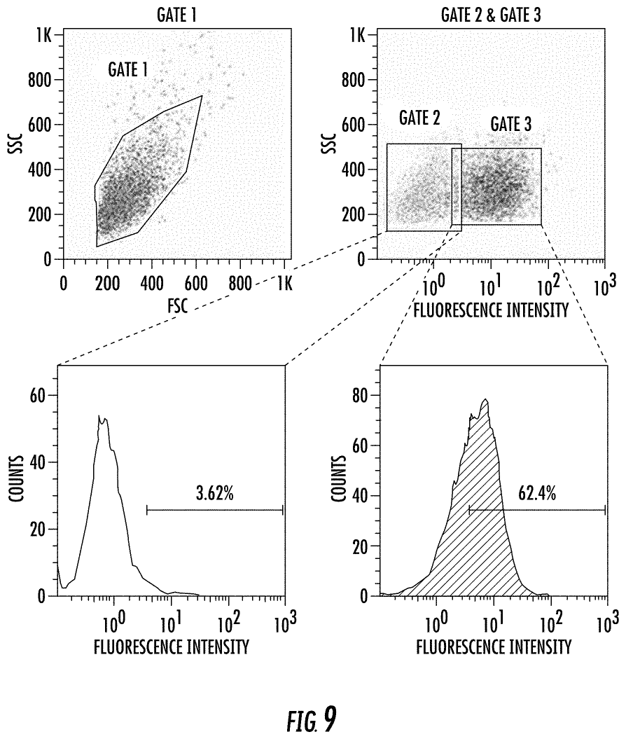

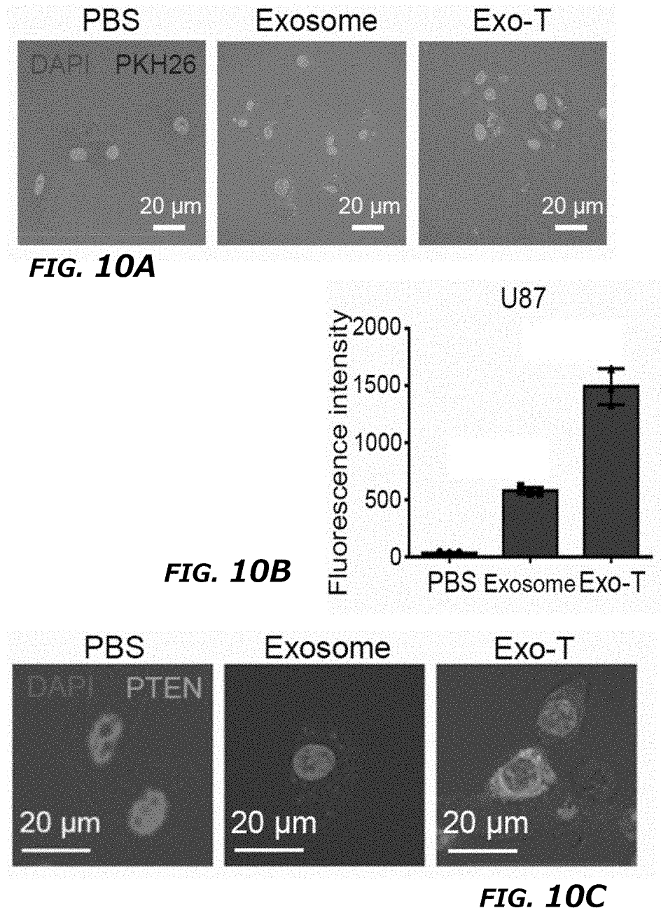

Commonly mutated tumor suppressor gene PTEN in a PTEN-deficient human U87 and a murine GL261 glioma model were evaluated for clinical utility of mRNA-exosomes. To achieve glioma targeting capabilities, glioma-targeting peptides were cloned into the N-terminus of CD47, a transmembrane protein abundant on the surface of exosomes (FIG. 8A). Two different peptides, a CDX peptide (FKESWREARGTRIERG (SEQ ID NO: 104)) for U87 targeting, and a CREKA for GL261 targeting, and a FLAG epitope, were inserted into the N-terminal of CD47, separately. Since the topology of CD47 on exosomes was unclear, a pulldown assay using anti-FLAG beads was performed to confirm that the N-terminal of CD47 was localized to the external exosomal surface (FIG. 8B). The addition of targeting peptide dramatically increased the CD47-exosome (Exo-T) uptake in U87 and GL261 cells, as well as translation of PTEN protein (FIG. 8C-F, FIG. 9, and FIG. 10A-D). Staining of endocytic markers revealed that the Exo-T co-localized strongly with transferrin (FIG. 8G and FIG. 10E). Inhibition of clathrin-mediated endocytosis significantly reduced the cellular uptake of exosomes (FIG. 8H and FIG. 10F), suggesting that Exo-T entry into target cells is likely mediated by clathrin-mediated endocytosis. Exo-T was able to inhibit tumor cell proliferation and exhibited minimal cellular toxicity (FIG. 8I-J and FIG. 10G-H).

To investigate the potential role of Exo-T for in vivo applications, pharmacokinetic properties OF Exo-T were evaluated. CD47 was strongly expressed on the exosome surface after cells were transfected with CD47 plasmid (FIG. 8K, upper panel). The overexpression of CD47 on the exosome surface was found to increase the in vivo circulatory half-life of Exo-T by 3-fold, while the addition of targeting peptide did not have any obvious effects on CD47 function (FIG. 8K) Immunogenicity results showed that Exo-T had no obvious in vivo toxicity and immunogenicity in mice at different dosages and different time points (2 h, 8 h, and 24 h) tested (FIG. 8L and FIG. 10I).

### Example 6. In Vivo Therapeutic Efficacy of Exo-T in Preclinical Models of Glioma

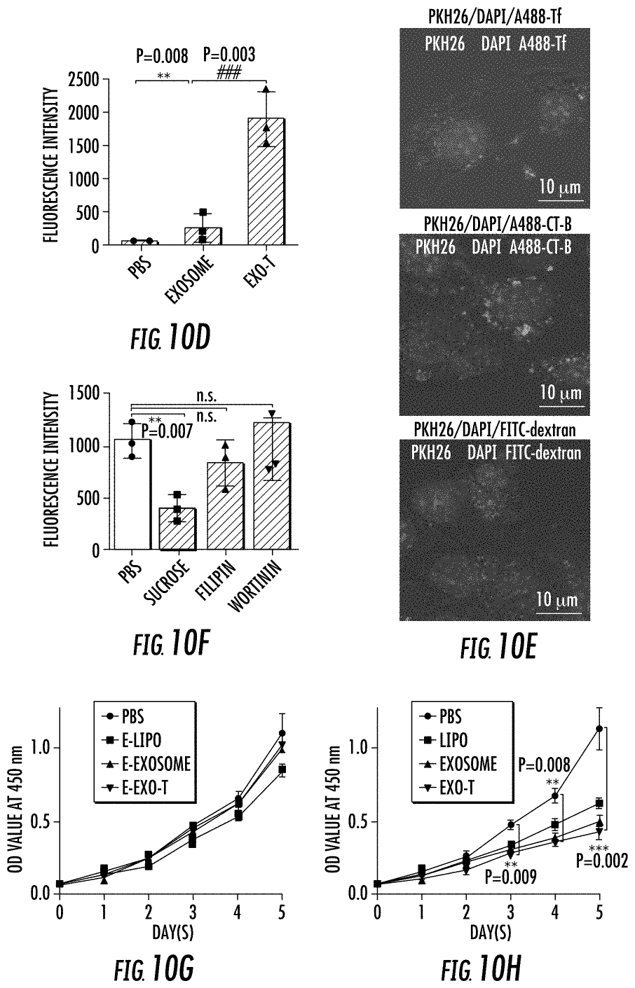

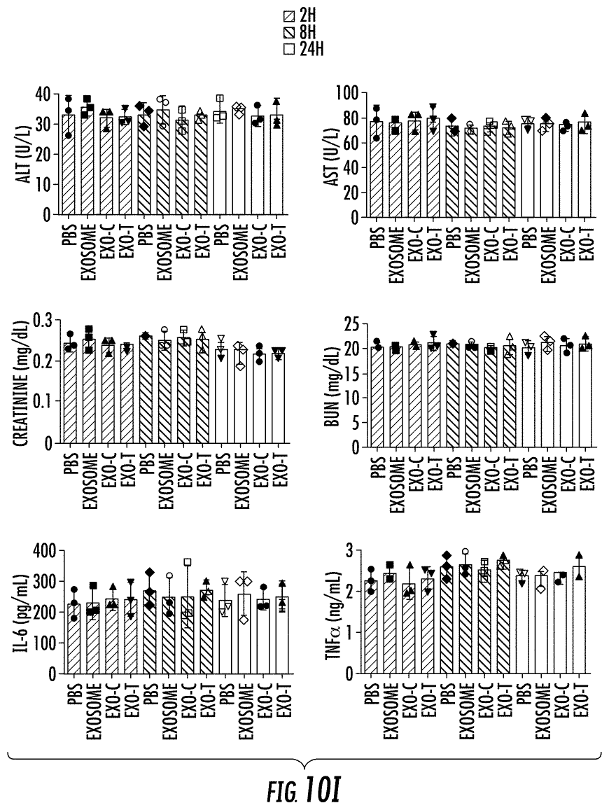

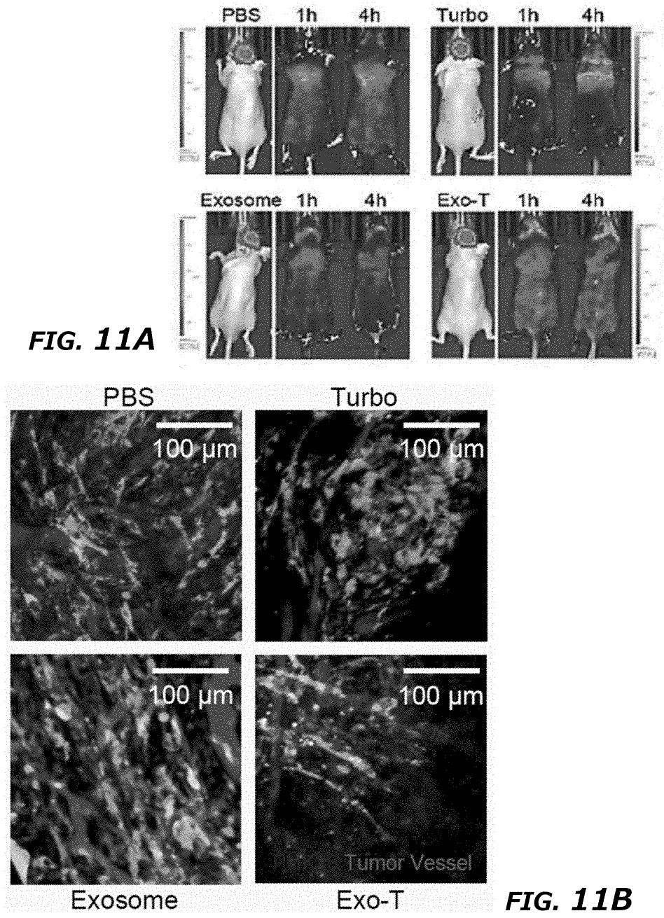

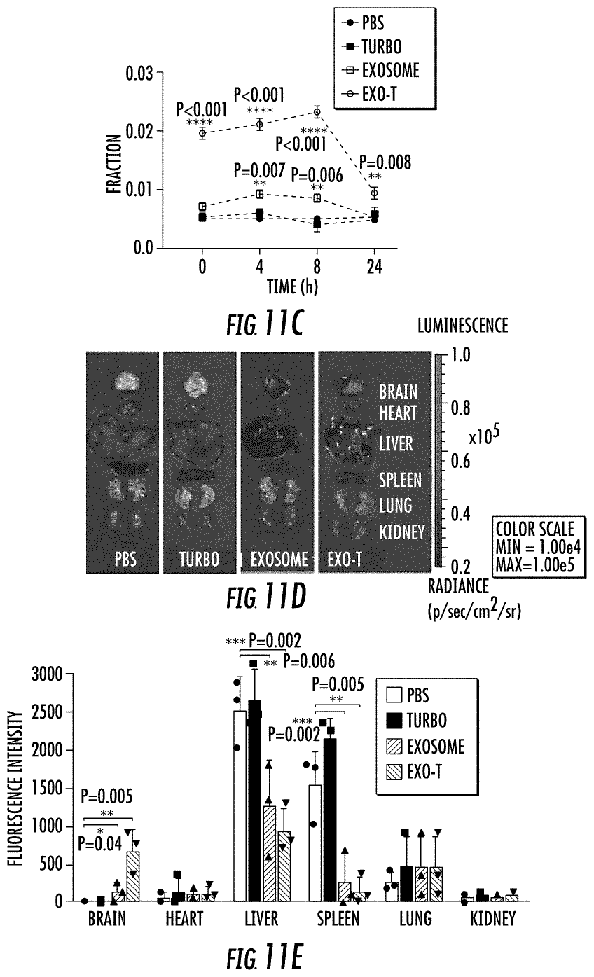

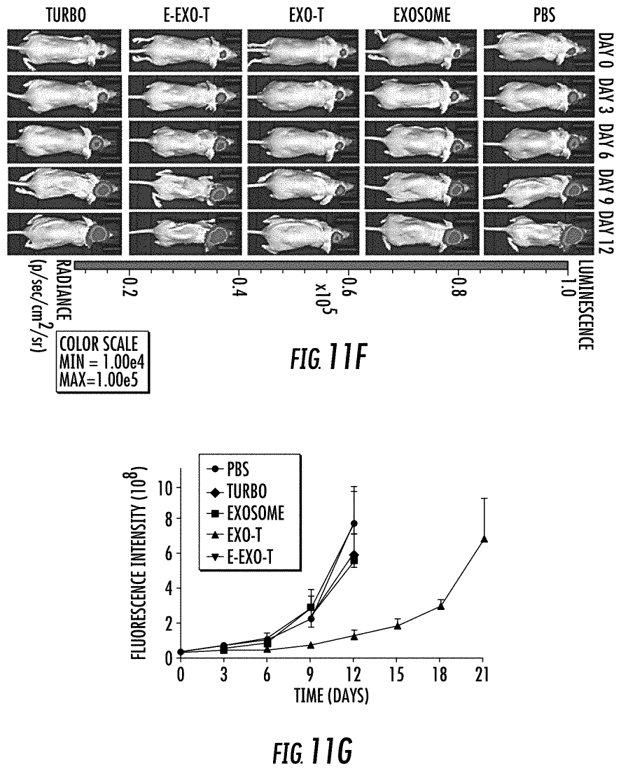

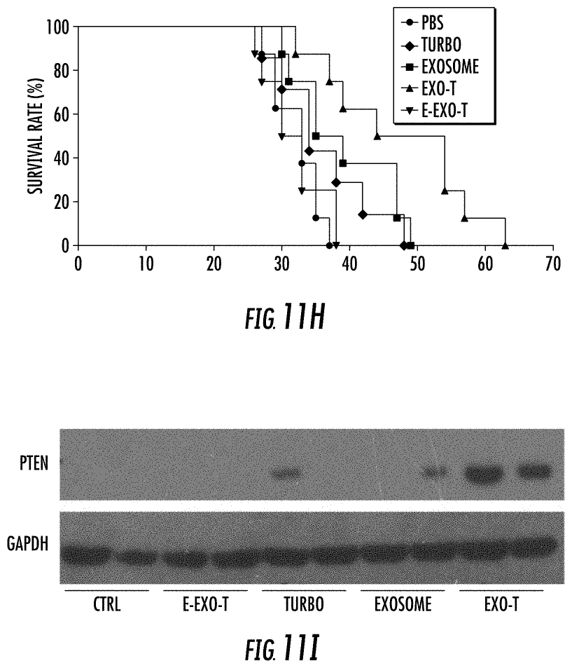

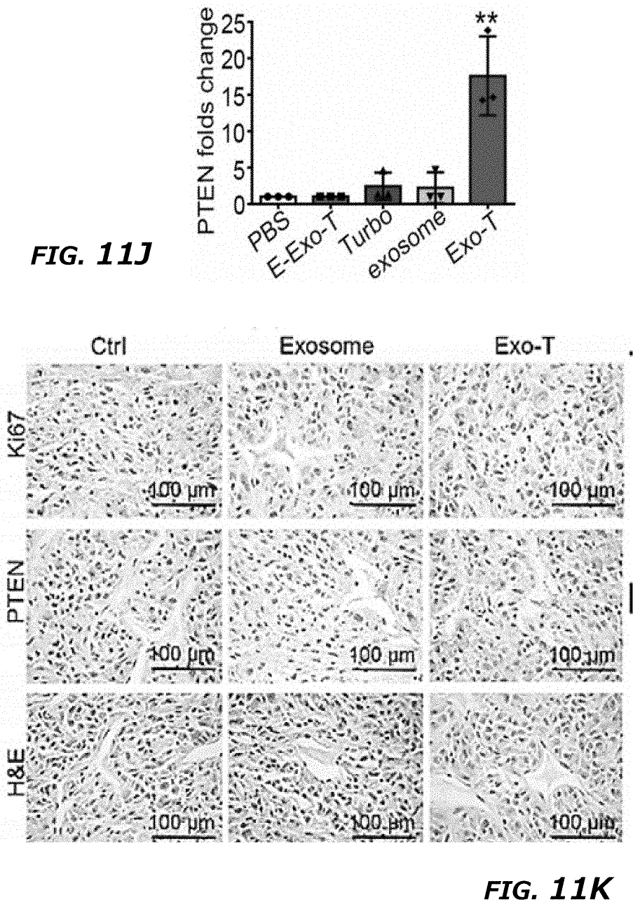

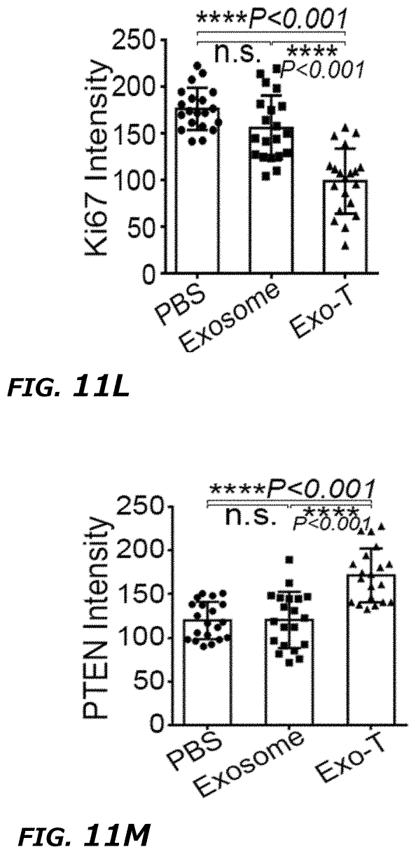

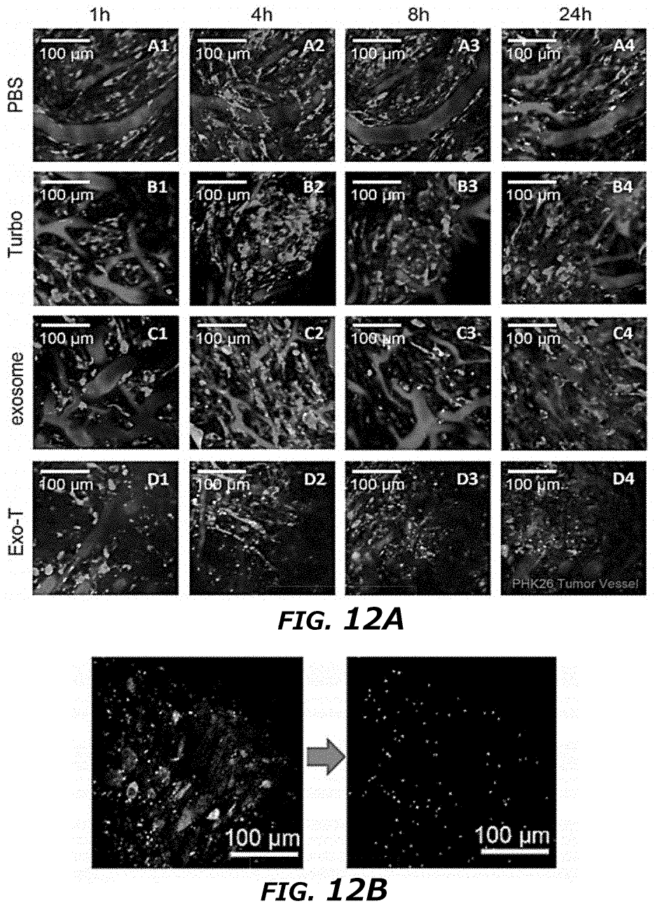

To assess the therapeutic potential of Exo-T in PTEN-deficient glioma models, Exo-T was intravenously injected into orthotopically implanted human U87 glioma-bearing immunodeficient mice. Compared to non-targeted exosomes (exosome) or TurboFect (Turbo) nanoparticles, Exo-T exhibited significantly improved tumor accumulation (FIG. 11A). To further evaluate the in vivo biodistribution of Exo-Ts within the tumor interstitium, PKH26-labelled Exo-T, exosome, and Turbo were administered systemically in tumor bearing mice and imaged with intravital fluorescence microscopy. A strong PKH fluorescence was observed within the tumor stroma 4 h after administration of Exo-Ts, but not for exosomes or TurbFect nanoparticles (FIG. 11B-C and FIG. 12A-B). Evaluation of systemic distributions further revealed a marked reduction in hepatic and splenic accumulation of Exo-Ts (FIG. 11D-E).

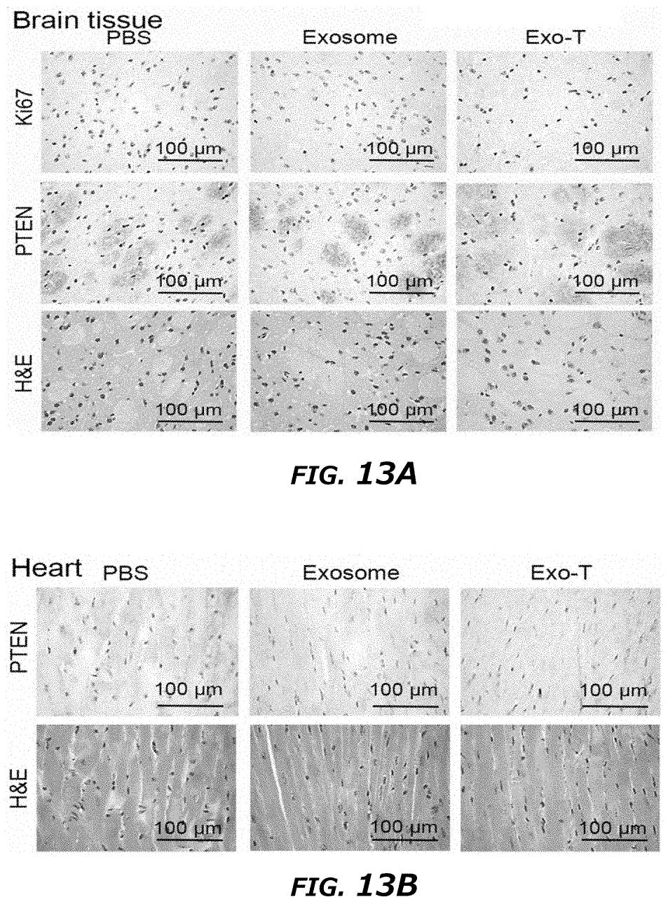

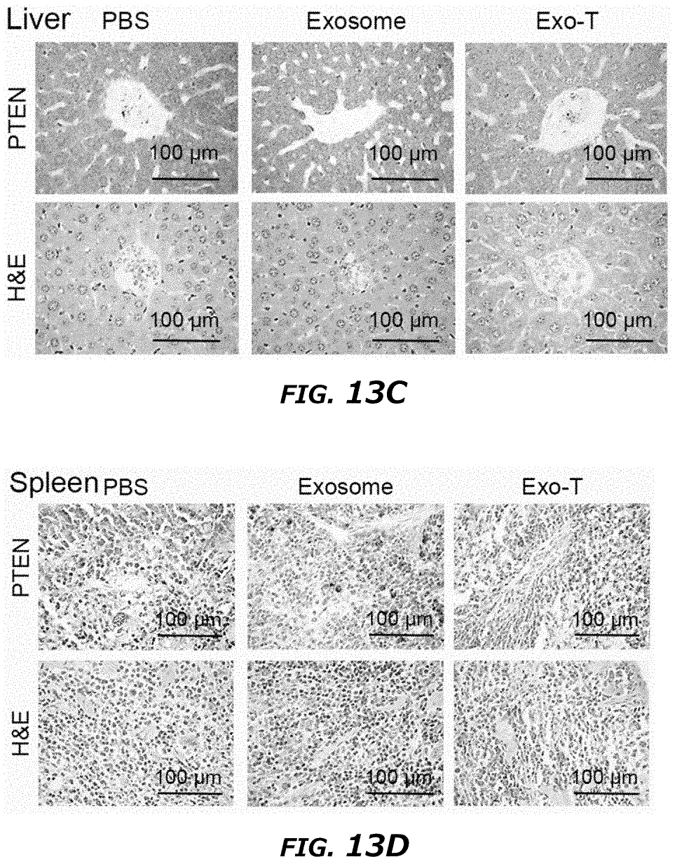

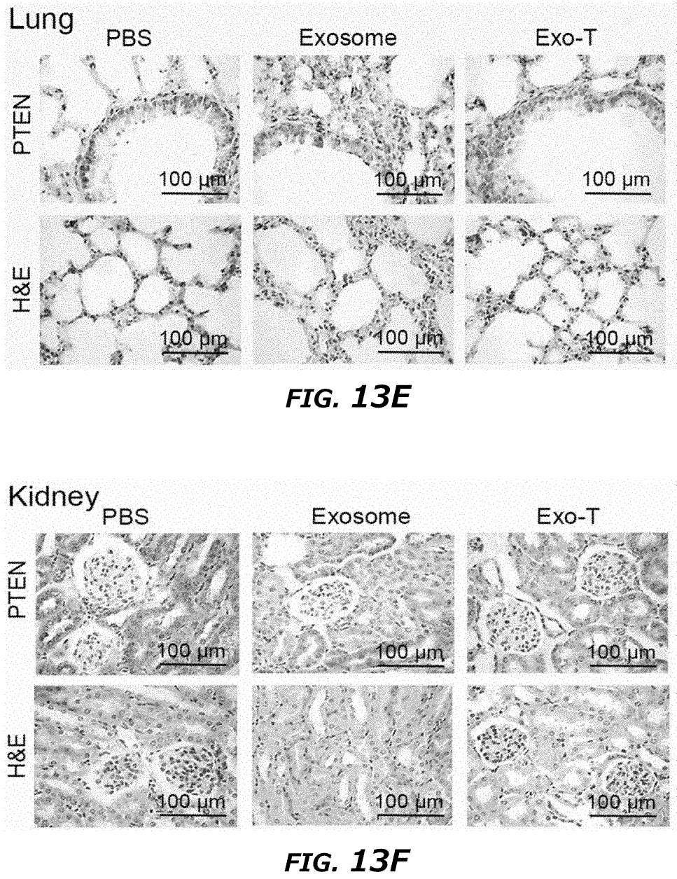

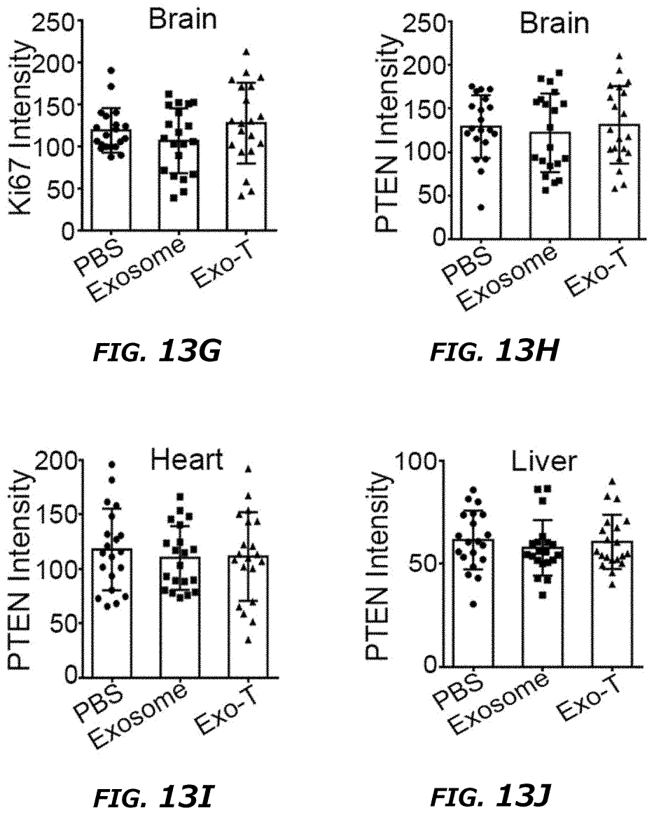

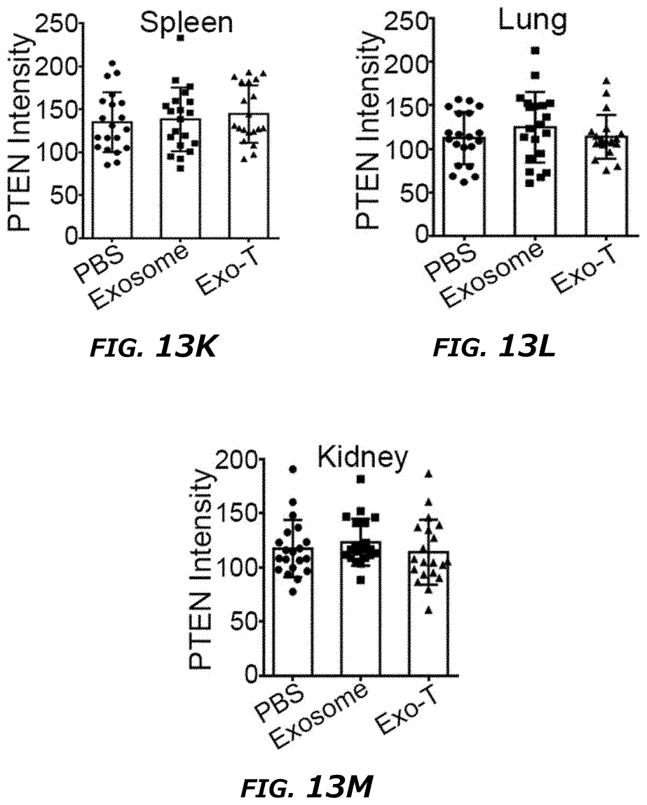

U87 mice treated with Exo-Ts demonstrated a significant inhibition in tumor growth (FIG. 11F-G) and exhibited prolonged survival with a median survival of 49 days as compared to 37 days for non-targeted exosomes (FIG. 11H). Evaluation of residual tumor tissue from both groups revealed that both PTEN mRNA and proteins levels were up-regulated after Exo-T treatment (FIG. 11I-J) Immunohistochemical staining results further confirmed that Exo-T treatment restored PTEN expression and inhibited tumor cell proliferation with no direct effect on other tissues examined (FIG. 11K-M and FIG. 13).

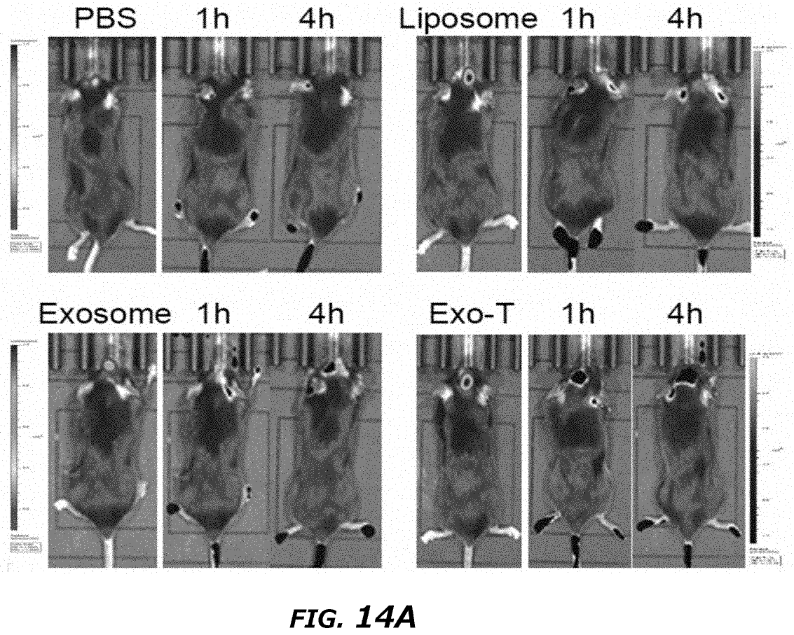

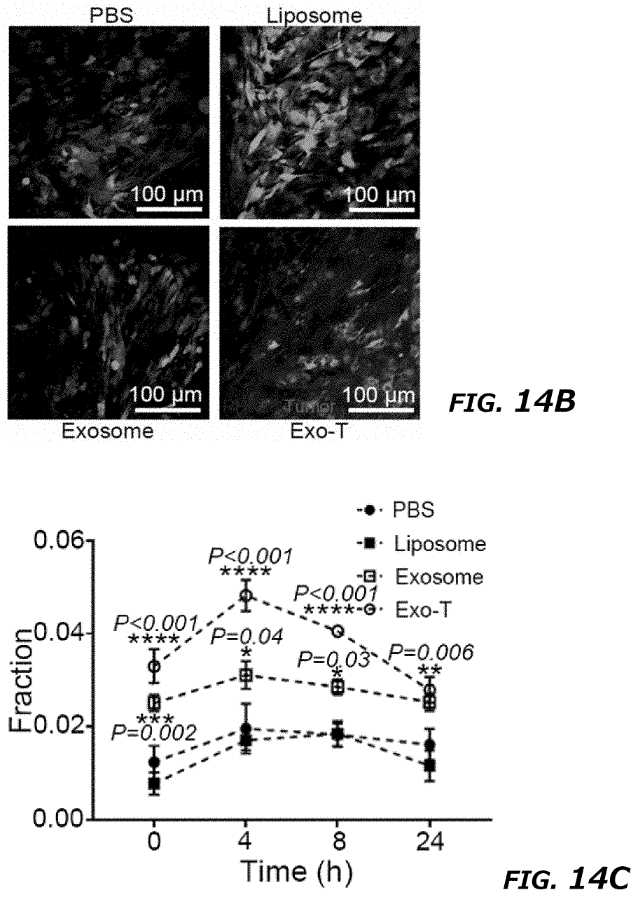

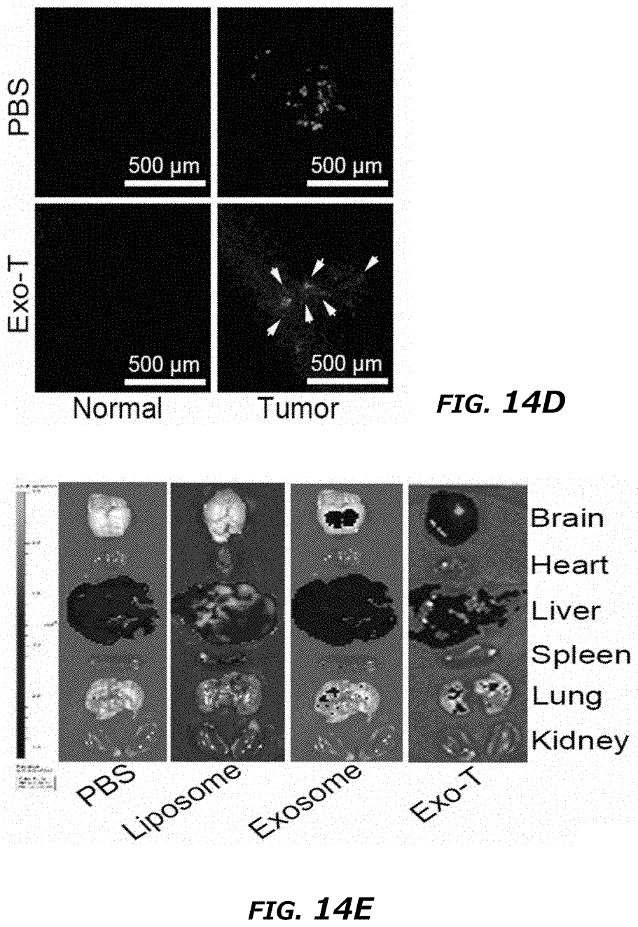

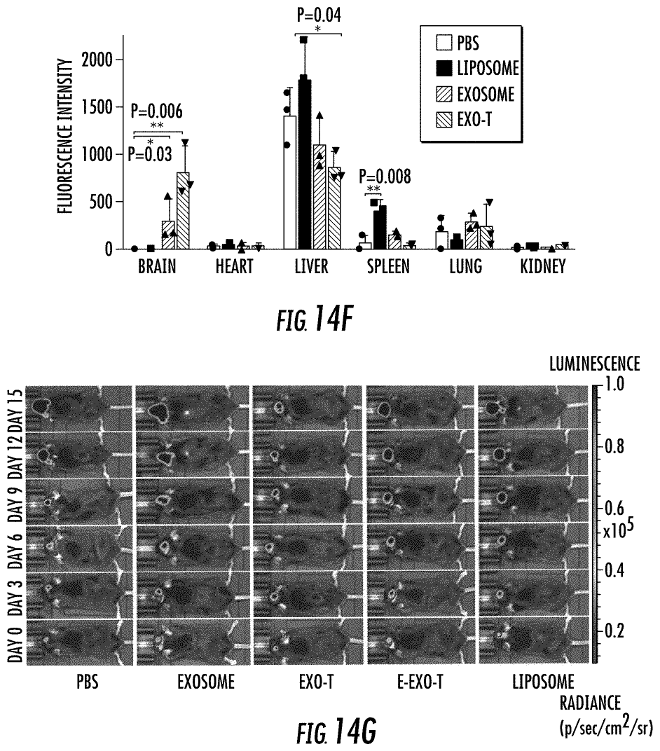

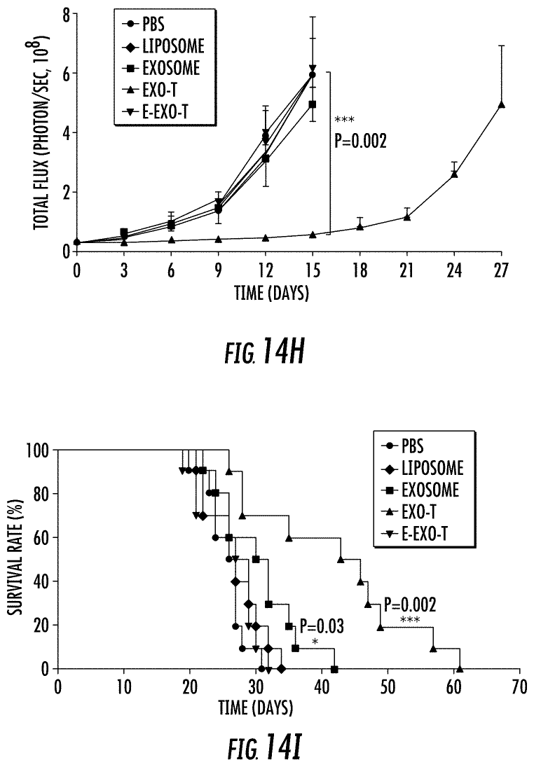

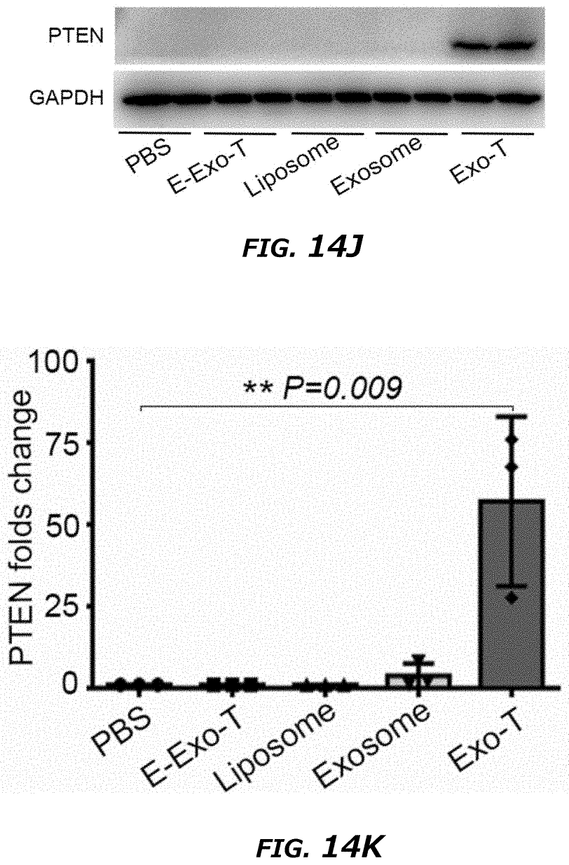

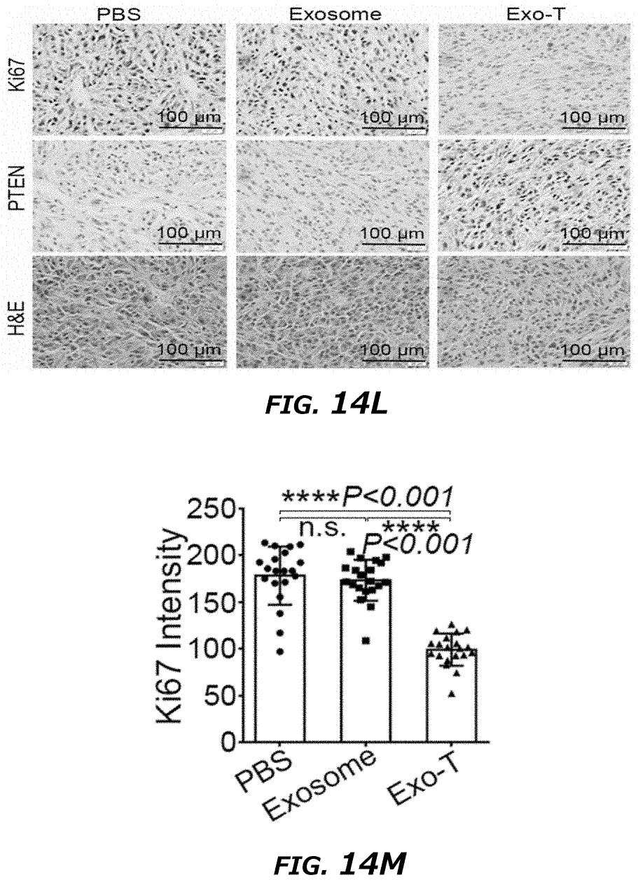

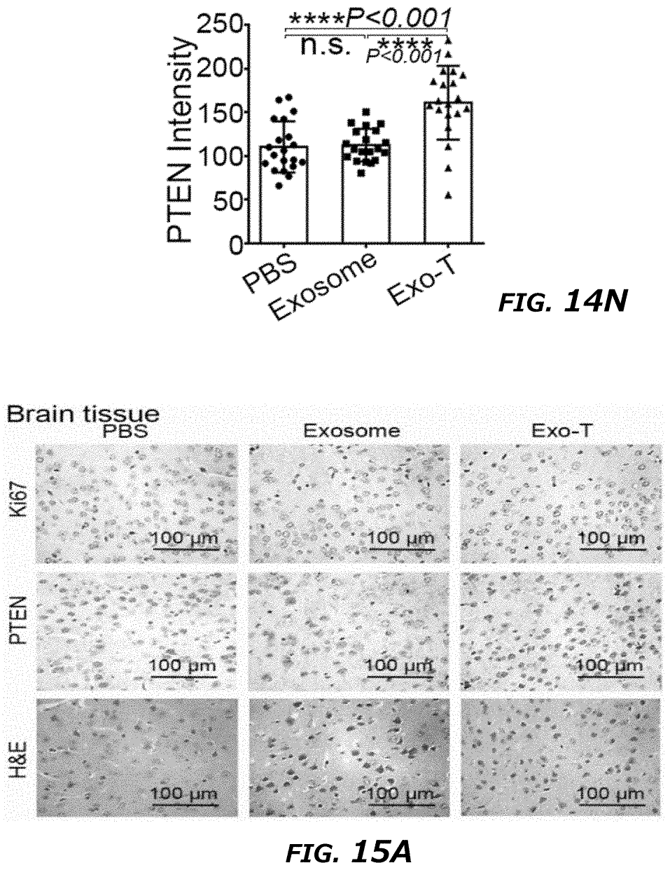

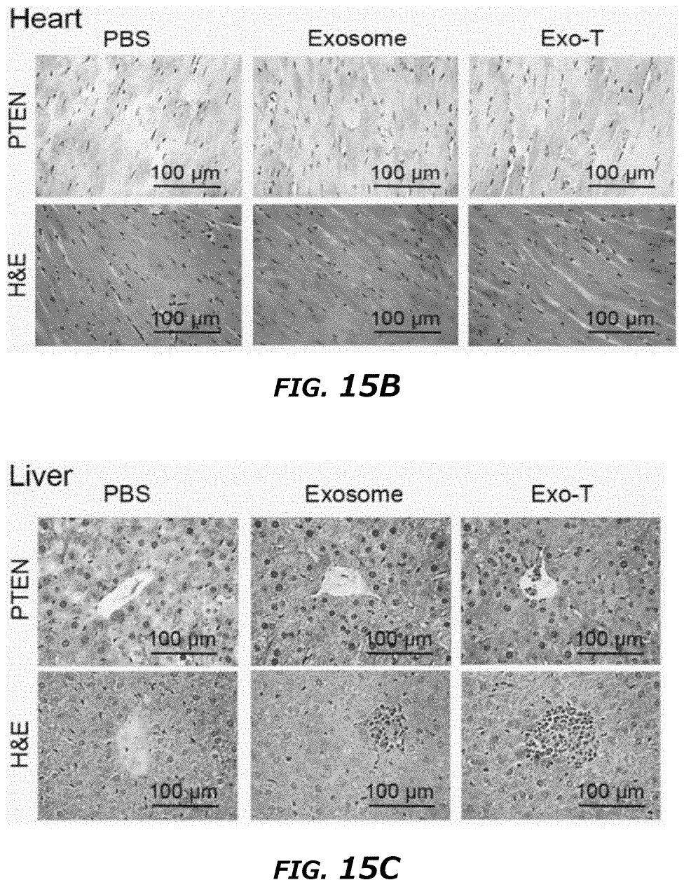

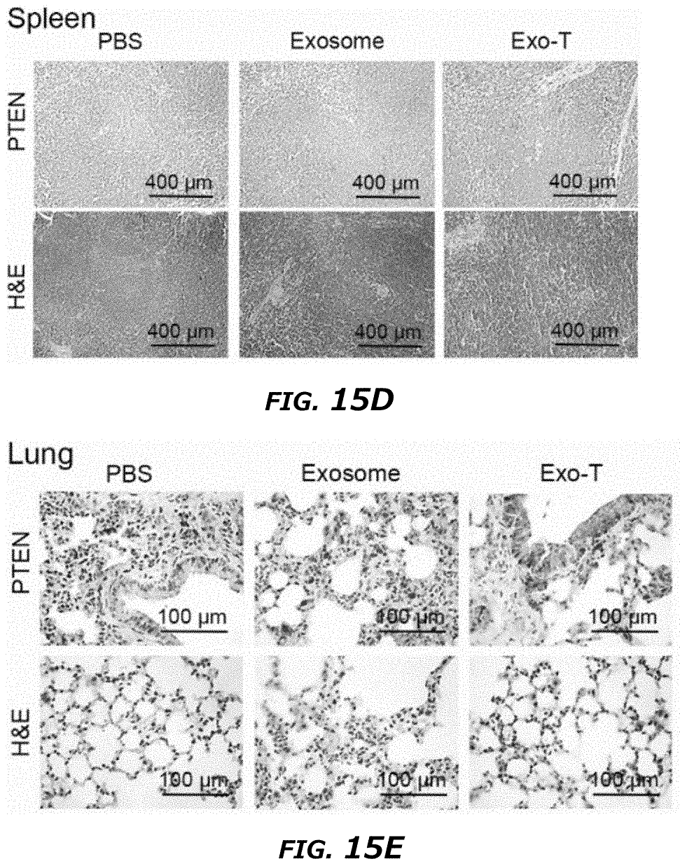

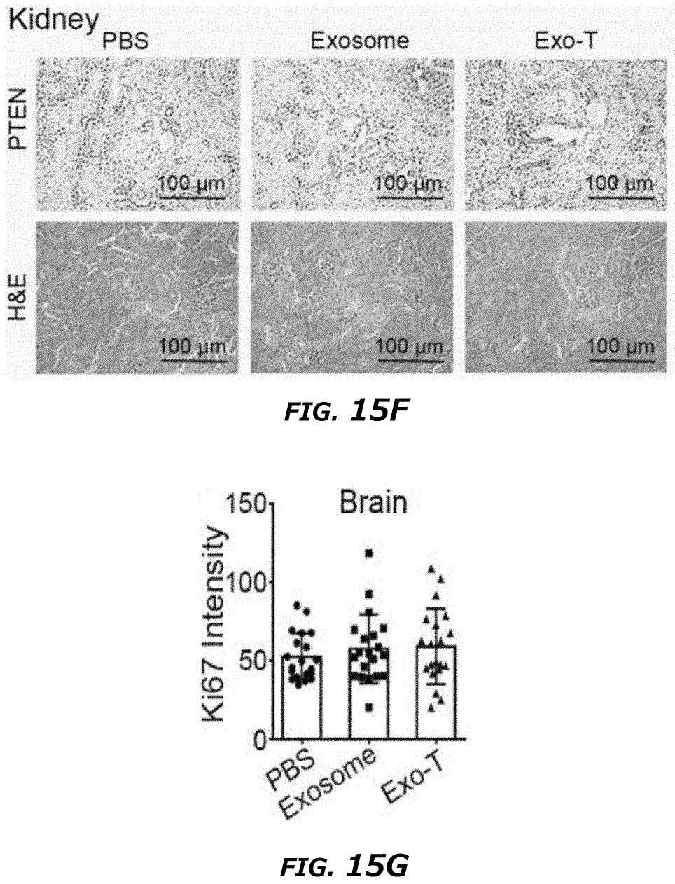

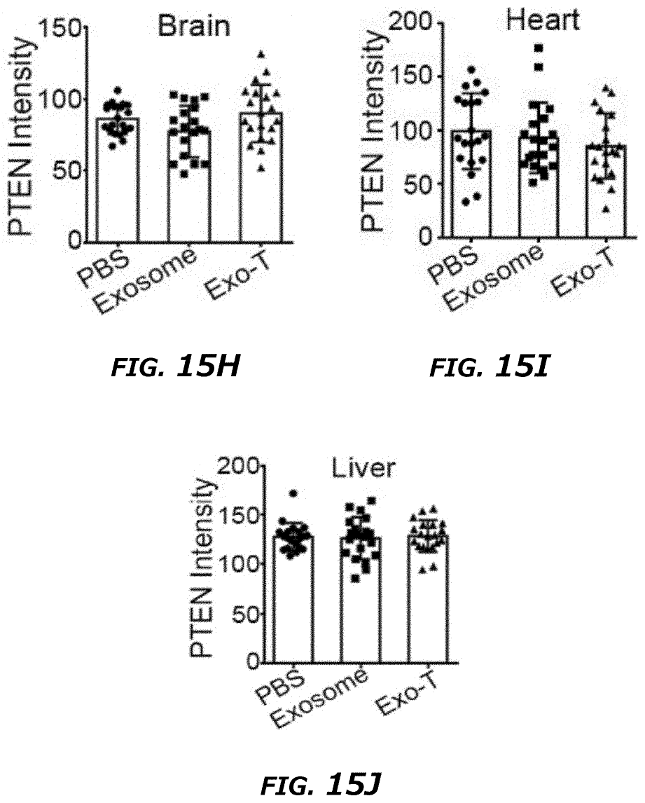

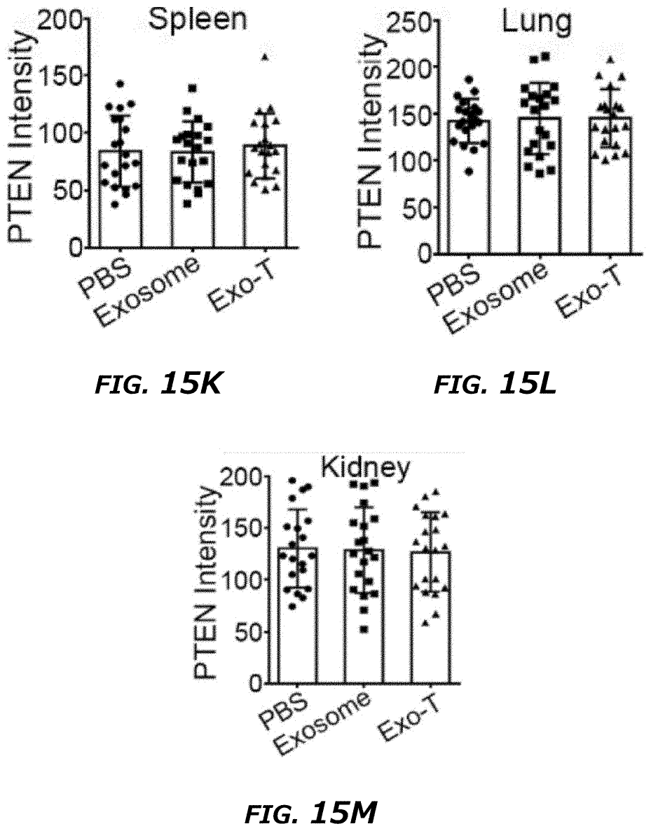

Next, the therapeutic efficacy of Exo-Ts in immune-competent PTEN-deficient GL261 glioma model was examined Compared to non-targeted exosomes (exosome) or PEG-liposome (Liposome), Exo-T exhibited significantly improved tumor accumulation (FIG. 14A). To further evaluate the in vivo biodistribution of Exo-Ts within the tumor interstitium, PKH26-labelled Exo-T, exosome, and PEG-liposome were administered systemically in tumor bearing mice and imaged with intravital fluorescence microscopy. A strong PKH fluorescence was observed within the tumor stroma 4 h after administration of Exo-Ts, but not for the exosomes cohorts or liposome nanoparticles cohorts (FIG. 14B-C). Ex vivo results showed that the majority of exosomes were taken up by brain tumor cells, whereas normal brain cells showed minimal exosome uptake following administration (FIG. 14D). Evaluation of systemic distribution further revealed a marked reduction in hepatic and splenic accumulation of Exo-Ts (FIG. 14E-F). GL261 mice treated with Exo-Ts demonstrated a significant inhibition in tumor growth (FIG. 14G-H) and experienced prolonged survival with a median survival of 45 days versus 31 days for non-targeted exosomes (FIG. 14I). Evaluation of residual tumor tissue from both groups revealed that both PTEN mRNA and proteins levels were up-regulated after Exo-T treatment (FIG. 14J-K) Immunohistochemical staining results further confirmed that Exo-T treatment restored PTEN expression and inhibited tumor cell proliferation with no direct effect on other tissues examined (FIG. 7L-N and FIG. 15).

### Example 7. Methods and Systems of Synthesizing Therapeutic Extracellular Vesicles

Described herein are exemplary methods and systems for producing extracellular vesicles or exosomes for encapsulating therapeutic polynucleotides. The extracellular vesicles and exosomes produced by the instant methods and systems can be suitable to be formulated into a pharmaceutical composition for therapeutic uses.

### Cell Culture

Mouse embryonic fibroblasts (MEFs) were cultured in Dulbecco's Modified Eagle's Medium (DMEM) containing 10% Heat-Inactivated Fetal Bovine Serum (FBS) and 1% Non-Essential Amino acid (NEAA). Human glioma U87-MG and HEK 293T cell lines were cultured in DMEM supplemented with 10 mmol/L HEPES, 10% FBS and 1% penicillin/streptomycin at 37° C. in humidified conditions equilibrated with 5% CO₂.

### Plasmid Preparation

Achaete-Scute Complex Like-1 (Ascl1), Pou Domain Class 3 Transcription factor 2 (Pou3f2 or Brn2) and Myelin Transcription Factor 1 Like (Myt1l) were synthesized. PTEN, CD47, CD63-GFP and miR-128 plasmids could be purchased. Primers designed to encode CDX (FKESWREARGTRIERG (SEQ ID NO: 105)), CREKA, and FLAG tag were used to introduce the ligands into the N-terminal of CD47. Isolation of mononucleocytes from mouse bone marrow. 4-12 weeks old C57BL/6 mice were sacrificed by cervical dislocation, and disinfected in 70% ethanol for 5 min. Femurs and tibiae were removed and purified under sterile conditions. The intact bones were then washed with PBS, and both ends were cut with scissors. Bone marrow was rinsed out with RPMI-1640 medium using 0.45 mm diameter needle. The cells were collected by centrifugation at 1,000 rpm for 5 min. The Tris-NH₄Cl red blood cell lysis buffer was added to the cell pellet to remove the red blood cells. The cell suspension was further centrifuged at 1,000 rpm for 5 min to collect the mononucleocytes. Induced culture of bone marrow-derived DCs (BMDCs). Isolated mononucleocytes were culture in RPMI-1640 medium supplemented with 10% FBS at 37° C. in an incubator containing 5% CO₂. The culture medium was supplemented with 20 ng/ml GM-CSF and 10 ng/ml IL-4. The unattached cells were removed 12 h after culture and replaced with fresh complete medium containing GM-CSF and IL-4. At day 7, the loosely attached cells were harvested by gently pipetting the medium against the flask. The cells were plated into CNP chips for additional incubation with lipopolysaccharide for 24 h.

Cell Transfection

For CNP, a single layer of MEFs, MSCs, DCs, or HEK-293T cells (−200,000 cells) was spread on a 1 cm×1 cm 3D CNP silicon chip surface for overnight cell incubation. Plasmids pre-loaded in PBS buffer were injected into individual cells via nanochannels (−500 nm diameter and 5 μm spacing) using a 200 V electric field for 5 pulses at 10 ms/pulse with a 0.1 sec interval. Various electroporation conditions were tested for best choice. Bulk electroporation (BEP), Gene Gun, and Lipofectamine 2000 transfections were conducted. Ascl1/Brn2/Myt1l plasmids at a weight ratio of 2/1/1 and a concentration of 100 ng/mL in PBS buffer were pre-mixed for transfection. Cell transfection of PTEN, miR-128, CD47, CDX-CD47, CREKA-CD47 and CD63-GFP plasmids followed the same procedure.

### Collection and Purification of EVs Secreted by Donor Cells

Cells were cultured in culture medium containing serum. Before transfection, the cell culture medium containing serum was removed. The cells were washed with PBS three times and cultured in serum-free cell culture medium. For qRT-PCR, EVs were collected from cell culture supernatants by centrifugation at 1500 g for 10 min. For EV particle measurements by dynamic light scattering goniometry (DLS) and NanoSight™ in vitro cell transfection and in vivo animal experiments, EVs were collected from cell culture supernatants by a series of centrifugation and ultracentrifugation steps.

### CNP Biochip Fabrication

Nanochannel array devices were fabricated based on double-polished 4-inch (100 mm) wafers. Briefly, a thin layer of Shipley 1813 photoresist was first spin-coated on the silicon wafers at 3,000 RPM after HMDS prime process. Nanopore openings on the photoresist were patterned using projection lithography. A deep reactive ion etching (DRIE) technique, “Bosch Process”, was utilized to etch a high-aspect-ratio (>20:1) nanochannel array (10 μm deep). An alternating sequence of etching gas SF6 and sidewall passivation gas C4F8 were set using optimized parameters. Microchannel reservoirs on the other side of the wafers were generated using a similar process combining photolithography and DRIE. Processed wafers were cleaned in piranha cleaning (120° C., 10 min) before they were diced into 1 cm×1 cm pieces. The PDMS spacers were made from a pre-polymer/curing agent mixture (10:1 weight ratio) cured at room temperature for 3 days. The PDMS and silicon surfaces were pre-treated with oxygen plasma to secure a tight bonding. A thin film of gold was deposited on a glass slide as a bottom electrode. A gold rod was used as the top electrode.

Sorting of Exosomes and Microvesicles from Total EVs

For the collected total EVs, microvesicles were sorted by centrifugation at 10,000 g for 30 min. The supernatant was further centrifugated at 100,000 g for 2 h to collect the smaller exosomes.

### EV Size Measurements by DLS and NanoSight™

Size distributions of EVs were determined using a DLS goniometer. Absolute numbers of exosomes and microvesicles secreted per cell were quantified by NanoSight™ using the same number of living donor cells after transfection for comparison.

### Agarose Gel Assay

The samples were loaded onto a 1% (w/v) agarose gel containing 0.5% μg/ml ethidium bromide. Electrophoresis was performed at 100 V for 30 minutes. The gel was imaged under UV light.

qRT-PCR of EV-Containing RNA Expression Levels

The expression of Ascl1, Brn2, Myt1l and PTEN mRNAs and miR-128 in EVs was measured using qRT-PCR. Briefly, total RNAs were obtained using TRIzol reagent. Reverse transcription of equal amounts of RNA was carried out using first-strand cDNA synthesis kit with random hexamers as primers. The expression of genes was measured using the CYBR green. All experiments were performed in triplicate. The primer sequences used were as follows:

Ascl1(mouse),
forward:
(SEQ ID NO: 106)
5′-TGGTGTCTGAACCTAAGCCC-3,
and
reverse:
(SEQ ID NO: 107)
5′-GTCCGAGAACTGACGTTGCT-3′;
Myt1l(mouse),
forward:
(SEQ ID NO: 108)
5′-CCTATGAGGACCAGTCTCC-3′,
and
reverse:
(SEQ ID NO: 109)
5′-GACATGGCTGTCACTGGAT-3′;
Brn2 (mouse),
forward:
(SEQ ID NO: 110)
5′-GACACGCCGACCTCAGAC-3′,
and
reverse:
(SEQ ID NO: 111)
5′-GATCCGCCTCTGCTTGAAT-3′;
GAPDH (mouse),
forward:
(SEQ ID NO: 112)
5′-GGGAAATTCAACGGCACAGT-3′
and
reverse:
(SEQ ID NO: 113)
5′-AGATGGTGATGGGCTTCCC-3′;
PTEN,
forward:
(SEQ ID NO: 114)
5′-CAAGATGATGTTTGAAACTATTCCAATG-3′,
and
reverse:
(SEQ ID NO: 115)
5′-CCTTTAGCTGGCAGACCACAA-3′;
and
GAPDH,
forward:
(SEQ ID NO: 116)
5′-GACAGTCAGCCGCATCTTCT-3′,
and
reverse:
(SEQ ID NO: 117)
5′-TTAAAAGCAGCCCTGGTGAC-3′.

### In Vitro Protein Translation

A same amount of total RNA (1 μg) from each transfection method was applied for in vitro protein translation. After the translation procedure was accomplished, samples were separated by SDS-PAGE and the proteins were detected with various antibodies as shown in the Western blotting plot.

### Transmission Electron Microscopy (TEM)

Cells for TEM analysis were collected 4 h after CNP transfection, re-suspended in 20% BSA in PBS, and then placed into a 200 μM deep hat and frozen at high pressure. Frozen samples were then freeze-substituted in 1% Osmium tetroxide and 0.1% uranyl acetate in a cold block allowed to warm in a Styrofoam block for 3 h to 0° C. and moved to a hood for 30 min, and held for 12 h then warmed to 25° C. in 5 h at 5° C./h and held around 12 h. The samples were washed twice in acetone and once in propylene oxide (PO) for 15 min each. Samples were infiltrated with resin mixed 1:2, 1:1, and 2:1 with PO for 2 h each with leaving samples in 2:1 resin to PO overnight rotating at room temperature in fume hood. The samples were then embedded and orientated with specimen carrier/cells (if still in hat) facing up and placed into 65° C. oven overnight. Sections were taken between 75 and 90 nm, picked up on formvar/Carbon coated 100 mesh Cu grids, then contrast stained for 30 sec in 3.5% uranyl acetate in 50% acetone followed by staining in 0.2% lead citrate for 3-4 min. Samples were observed and photos were taken using a 2 k×4 k digital camera.

### Cryo-TEM

For cryo-electron microscopy, 3 μl of EV sample was applied onto a glow-discharged 300-mesh R2.0/2.0 Quantifoil grid. The grid was blotted by Whatman #1 filtration paper and rapidly frozen in liquid ethane using a Vitrobot IV plunger (FEI). Micrographs were recorded on a 4 k×4 k CCD camera at a magnification of 59,000×, a dose of 20 electrons/Å2, and a defocus of 5 μm in a FEI Tecnai F30 electron microscope operated at 300 kV.

### Cell Membrane Damage Evaluated by Cell Uptake of Propidium Iodide (PI)

CNP-induced transient cell membrane damage was quantified by diffusion-based cell uptake of PI ( ) and subsequent fluorescent signal. MEFs were transfected by CNP with a 200 V, 10 ms electric pulse. PI was immediately added in either top (cell side) or bottom reservoir. On-chip time-lapse epi-fluorescence live cell imaging was conducted using an inverted microscope system equipped with EMCCD camera. BEP of MEFs was also done as a control following the electric field conditions. Exemplary BEP protocols and conditions can include manual from BEP supplier Neon Transfection System.

### Measurement of Intracellular Calcium Concentration

Cells were incubated with 10 μM Fura2-Am at 37° C. for 1 hour. The extracellular dye was washed away with PBS, and cells were resuspended in complete RPMI medium. The fluorescence changes after the addition of 7 μM MON were recorded in a fluorescence spectrophotometer. All the experiments were protected from light and completed within 2 hours.

### Temperature Measurement During CNP

Temperature rise by joule heating during CNP was measured using a temperature-sensitive fluorescent dye, Rhodamine B. To prevent fluorescence dye diffusion, sodium alginate solution was added with calcium chloride powder to form calcium alginate gel to suppress the dye diffusion during CNP.

### FEM Heat Transfer Simulation During CNP

The temperature field near a nanochannel was simulated using COMSOL® Multiphysics 5.0. (COMSOL Inc.) “heat transfer in fluids” module, by solving the governing COMSOL® Multiphysics 5.0. (COMSOL Inc.) “heat transfer in fluids” module, by solving the governing equation

$$ρ {c}_{p} \frac{\partial T}{\partial t} + ρ {c}_{p} u \cdot ∇ T = k {∇}^{2} T + Q ,$$

where ρ: density cₚ: heat capacity u: flow rate k: thermal conductivity; Initial temperature=22° C. The nanochannel is regarded as a pulsed heat source with a power density Q_{nc}=P/V≈10¹⁴ W/m³. The simulated data was exported to MATLAB (MathWorks) for analysis.

### EV Pulldown Assay

Protein-A Sepharose beads were incubated with 2 mg/ml BSA/PBS solution at 4° C. overnight. The beads were subsequently washed with cold PBS three times. Rabbit anti-FLAG antibody was incubated with beads at 4° C. for 4 h, and then washed three times with cold PBS. Purified EVs were incubated with the beads overnight. After washing, the beads were eluted in 0.1% SDS and 20 μl of the supernatant was used for the polyacrylamide gel.

### Cellular Uptake by Flow Cytometry

EVs were labeled with PHK67 and incubated with 60,000 U87-MG cells in a 24-well plate at 37° C. for 4 h prior to treatment. After incubation, cells were rinsed three times with cold PBS and fixed in 4% paraformaldehyde solution. The cell fluorescence intensity was analyzed by using a Beckman Coulter EPICS XL flow cytometer. A minimum of 10,000 events were collected for each cell sample under LIST mode.

### Confocal Microscopy

After incubation with PKH26 stained EVs for 4 h, cells were washed twice with cold PBS, and fixed with formaldehyde (4%)/PBS for 30 min. Cell nuclei were stained with DAPI with gold coating solution, and the fluorescence was visualized and recorded on a Laser Scanning Confocal Microscopy (LSM710, Carl Zeiss, Germany) All images were analyzed using a background subtraction method offline.

### Tethered Lipoplex Nanoparticle (TLN) Biochip Assay and Total Internal Reflection Fluorescence (TIRF) Imaging

EVs were tested using a tethered lipoplex nanoparticle (TLN) biochip on a total internal reflection fluorescence (TIRF) microscope (Nikon Eclipse Ti Inverted Microscope System. Briefly, a molecular beacon (MB) for the RNA target was encapsulated in cationic liposomal nanoparticles. These cationic lipoplex nanoparticles were tethered on a glass slide, which captured negatively charged EVs by electrical static interactions to form a larger nanoscale complex. This lipoplex-EV fusion led to mixing of RNAs and MBs within the nanoscale confinement near the biochip interface. TIRF microscopy was used to measure the fluorescence signals within 300 nm range of focal plane interface, which was where the tethered liposomal nanoparticles located.

### MTS Assay

U87-MG cells were seeded at a density of 5000 cells/well in a 96-well plate 24 h prior to transfection. Cells were washed three times with serum-free media and incubated with EVs. At 48 h post-transfection, the media was replaced with fresh cell culture media. Cell viability was then analyzed by MTS assay per manufacturer's instructions. Briefly, 20 μl of the MTS reagent (Promega) was added to each well. After incubation of the microplate in a humidified atmosphere (5% CO₂, 37° C.) for 2 h, the spectrophotometrically absorbance was measured using a microplate reader. The measurement wavelength was set at 490 nm. Cell survival was presented as a percentage of the untreated control.

### Animal Study

BALB/C-nu and C57BL/6 mice 6-8 weeks old were kept in isolator cages in a pathogen-free facility. The mouse experimental protocols were approved by Scientific Investigation Board of Science & Technology of Jilin Province or Institutional Animal Care, and were conducted in accordance with the National Institute of Health Guide for the Care and Use of Laboratory Animals.

### In Vivo Toxicity and Immunogenicity Assay

In vivo toxicity assay by measuring ALT, AST, BUN, and creatinine in serum of wild type C57BL/6 mice after systemic delivery of EVs was performed. Serum levels of IL-6 and TNF in mice after injection of EVs were measured by the IL-6 and TNF alpha ELISA kits.

### Animal Surgery and Tumor Implantation

Mice (BALB/c-nu or C57BL/6 6-8 weeks old, male) were anesthetized by the intraperitoneal injection of 10% chloral hydrate and immobilized in the stereotactic apparatus. After anesthesia, dexamethasone (2 mg/kg) and buprenorphine (0.2 mg/kg) were subcutaneously administered to reduce inflammation and pain. The head was shaved and the skull exposed. A circular craniotomy (diameter: 3-4 mm) was performed with a surgical drill above the somatosensory cortex. Tumor cells (1×108, U87-Luc tumor cells for BALB/c-nu mice, GL261-Luc tumor cells for C57BL/6 mice) were pressure injected into the cortex approximately 800 μm below the surface with a 32-gauge needle using micromanipulators at a rate of 0.1 μl/min using the following coordinates (the position of the injection is the caudate nucleus): 0.5 mm anterior and 1.5 mm lateral to bregma, at a depth of 3.0 mm from the brain surface. Following implantation, a round glass coverslip (diameter: 5 mm) was glued onto the surrounding craniectomy site and then further fixed with a dental cement. Body temperature was monitored by a rectal probe and maintained at 37° C. by a heating blanket. Dexamethasone (s.c., 2 mg/kg) and buprenorphine (s.c., 0.2 mg/kg) were administered once daily for one week to reduce post-surgical inflammation and pain. The animals were first imaged 14 days after tumor implantation and experiments were performed only if the physiological variables remained within normal limits.

### IVIS Imaging

BALB/C-nu or C57BL/6 mice were used to study the in vivo targeting and biodistribution of exosomes separately. 14 days after tumor implantation (1×108, U87-Luc tumor cells for BALB/c-nu mice, GL261-Luc tumor cells for C57BL/6 mice), the tumor was confirmed under fluorescence microscopy. PKH26-labeled Exo, Exo-T and TurboFect in vivo transfection or PEG-liposome were injected intravenously through the tail vein. 1 h and 4 h post injection, the mice were anesthetized by 10% chloral hydrate and recorded by IVIS Spectrum (PerkinElmer, Waltham, America). After 4 h, the mice were sacrificed, and major organs including brain, liver, lung, spleen, heart and kidney were collected. The fluorescence signals of PKH26 were captured and analyzed.

### Two-Photon Imaging

Mice (BALB/c-nu or C57BL/6, 6-8 weeks old, male) were anesthetized by the intraperitoneal injection of 3% chloral hydrate and immobilized in the custom-made stereotactic apparatus under the objective. Saline, nanoparticles (NPs) and EVs were mixed with PKH26 linker kits in a ratio of 1:1, respectively and the mixture was immediately injected intravenously into the four different groups: PBS, NPs (Turbo for BALB/c-nu mice and Liposome for C57BL/6 mice), exosomes and Exo-Ts; (n=3 per group). The upright laser scanning microscope attached to a Ti: sapphire pulsed laser system (80 MHz repetition rate, <100 fs pulse width, Spectra Physics) and software was used to track and measure the distribution of saline, NPs and EVs within tumor area at different time after injection: 1 h, 4 h, 8 h, and 24 h. 20× water immersion (NA, 1.00; WD, 2 mm, Olympus), and 40× water-immersion objectives (NA 0.80, WD; 3.3 mm, Olympus) were selectively chosen for fluorescence imaging in vivo, 890-nm irradiation wavelength was used to excite U87-Luc (or G1261-Luc) and PKH26 Red fluorescence, and emission light was differentiated and collected with 525/50 and 595/500 filters, respectively. The average laser power for imaging was less than 50 mW.

### Anti-Glioma Activity

10 days after tumor cell implantation, the tumor was confirmed under fluorescence microscopy. The mice were randomly divided into five groups, treated with saline, Exo, Exo-T, E Exo-T, Turbo (or Liposome) respectively. Formulations were administered via the tail vein once every three days and the dose was 1012 exosomes per mouse. Exosomes from MEFs were used for U87 animal model, and exosomes from BMDCs were used for GL261 animal model. The fluorescence signals of luciferase were captured and analyzed at 3, 6, 9, and 12 days separately.

### Histology and Immunohistochemistry (IHC) Analysis

All slides were deparaffinized in xylene 10 min for three times and rehydrated through graded ethanol. Antigen retrieval and immunostaining was performed as described previously 18 and a Vector M.O.M. Basic Kit was used. Briefly, antigen retrieval was carried out using 10 mM citrate buffer (pH=6.0). Slides were incubated in 0.3% hydrogen peroxide for 30 min to quench the activity of endogenous horseradish peroxidase and then blocked with TBST/5% normal goat serum. The primary antibody against PTEN or Ki-67 was used at 1:1000 dilution. Histological analyses on the other normal organs, including liver, lung, heart, spleen, and kidney, using H&E staining were performed. The intensities of PTEN and Ki67 in various groups were analyzed using image processing software.

### Statistical Analysis

Data are shown as mean±s.e.m. of triplicates unless otherwise indicated. Statistical analysis was performed using a two-tailed Student's t-test or one-way ANOVA with post-hoc tests, as appropriate. A P-value less than 0.05 was designated statistically significant.

### Example 8. Generation of mRNA-Encapsulating Exosomes by Cellular Nanoelectroporation

The exosomes described herein are attractive nucleic-acid carriers because of their favorable pharmacokinetic and immunological properties and of their ability to penetrate physiological barriers that are impermissible to synthetic drug-delivery vehicles. In the past, inserting exogenous nucleic acids, especially large messenger RNAs (mRNAs), into cell-secreted exosomes was challenging and may lead to low yields. Thus, the ability to produce large quantities of exosomes containing an abundance of endogenously-transcribed mRNAs is a major challenge. Described herein is a cellular-nanoporation method for the production of large quantities of exosomes containing therapeutic mRNAs and targeting peptides. Various source cells were transfected with plasmid DNAs, and stimulated the cells with a focal and transient electrical stimulus that promotes the release of exosomes carrying transcribed mRNAs and targeting peptides. Compared to bulk electroporation and to other exosome-production strategies, cellular nanoporation produced up to 50-fold more exosomes and more than a 10³-fold increase in exosomal mRNA transcripts, even from cells with low basal levels of exosome secretion. In the orthotopic PTEN-deficient glioma mouse models, mRNA-containing exosomes restored tumour-suppressor function, enhanced tumour-growth inhibition, and increased animal survival. Cellular nanoporation may enable the use of exosomes as a universal nucleic-acid carrier for applications requiring transcriptional manipulation.

Here, a non-genetic strategy to efficiently incorporate a high abundance of messenger RNAs (mRNAs) into exosomes for targeted transcriptional manipulation and therapy was described.

The study described herein demonstrates that through using a cellular nanoporation technique, large-scale production of exosomes containing endogenously transcribed mRNA from a variety of cellular sources could be achieved. Unlike other cell electroporation and stress inducing strategies, the controlled focal generation of transient membrane pores using nanochannels enables simultaneous delivery of plasmid DNA into source cells, which would likely not be possible with the simple administration of growth factors or cytokines.

Because the induction of membrane pores by the nanochannels is often helpful for stimulating cellular exosomal release, different nanochannel sizes, ranging from 100 to 1000 nm were investigated. It was found that for cells with diameters of approximately 10-20 μm, nanochannels within this size range are sufficient for plasmid delivery. When the channel diameter is larger than 1 μm, cell transfection mechanisms change because a much lower electric voltage (<50 V) is needed to avoid excessive cellular death. However, low voltage may cause the delivery of plasmids to be more inefficient. For the current study, the choice of a nanochannel with a diameter of 500 nm was based on its ability to sufficiently deliver plasmid DNA into the cells, without inducing cellular injuries that diminish the overall electroporation efficiency.

The results also suggest a mechanism by which cellular intrinsic processes can promote exosome generation and subsequent secretion in response to external stress. It was found that focal cell membrane injuries and local heating from CNP resulted in upregulated HSPs and elevated intracellular calcium [Ca²⁺], leading to the formation of a large number of intracellular vesicles. These vesicles are released as secreted exosomes, which can be induced to contain therapeutic RNAs after plasmid DNA delivery. The mechanism may possibly involve the influx of [Ca²⁺], which along with P53-TSAP6 activation as a part of the HSP response, promotes increased exosome production and subsequent secretion. The results provided herein suggest that a properly controlled CNP approach is not only effective for intracellular nucleic-acid delivery, but more importantly, it also stimulates intrinsic cellular adaptive processes to produce exosomes for therapeutic use. It is also worth noting that minimal cell death or activation of apoptosis pathways with CNP was observed, despite an increase in P53 expression. This may be explained by the transient and localized heat shock response at the nanopore site during CNP transfection.

One concern relating to the utilization of source cells to intrinsically encapsulate transcribed mRNA into secreted exosomes is mRNA loading efficiency. The experiments provided herein show that exosomes isolated from MEFs under normal physiological conditions contained minimal intact mRNA copies, with >99% of the exosomal RNAs having a size of less than 500 KD. It was estimated that on average, one intact mRNA can be found within every 10³ exosomes produced endogenously without external stimulation. In the setting of CNP treatment, the same source cells produced 2-10 intact mRNAs per exosome, which corresponds to a 2,000 to 10,000-fold increase in loading efficiency. Also, both step ultracentrifugation and Optiprep™ density gradient purification methods to purify exosomes from culture medium were tested. The mRNA recovery ratio for Optiprep™ density gradient purification is only about 10-20% of step ultracentrifugation, although a more concentrated RNA collection in the exosome fraction (Fraction 5-7) was observed. Moreover, chemicals involved in the separation process may be left behind. Therefore, given the similar mRNA rates, step ultracentrifugation in the therapeutic models described herein was selected.

By comparison, the CNP method as demonstrated in this study generally does not require any modifications to the source cells or target mRNA/protein sequences with minimal post-secretion processing of collected EVs required as compared to post-insertion electroporation.

While the foregoing disclosure has been described in some detail for purposes of clarity and understanding, it will be clear to one skilled in the art from a reading of this disclosure that various changes in form and detail can be made without departing from the true scope of the disclosure. For example, all the techniques and apparatus described above can be used in various combinations. All publications, patents, patent applications, and/or other documents cited in this application are incorporated by reference in their entirety for all purposes to the same extent as if each individual publication, patent, patent application, and/or other document were individually and separately indicated to be incorporated by reference for all purposes.

1. A method of producing an extracellular vesicle, said method comprising:
a) nanoelectroporating an extracellular vesicle donor cell with at least one polynucleotide, wherein said at least one polynucleotide encodes a targeting polypeptide that comprises: (i) an adapter polypeptide comprising a transmembrane domain and an extracellular domain; and (ii) a heterologous targeting domain that is covalently linked to said extracellular domain of said adapter polypeptide;
b) incubating said extracellular vesicle donor cell under conditions such that (i) said targeting polypeptide is expressed in said extracellular vesicle donor cell and (ii) said targeting polypeptide is incorporated into an extracellular vesicle released from said extracellular vesicle donor cell; and
c) collecting said at least one extracellular vesicle released from said extracellular vesicle donor cell, wherein said at least one extracellular vesicle comprises said targeting polypeptide.

2. The method of claim 1, wherein said heterologous targeting domain is covalently linked to a N terminus of said extracellular domain of said adapter polypeptide.

3. (canceled)

4. The method of claim 1, wherein said transmembrane domain of said adapter polypeptide is at least 70% identical to a transmembrane domain of a CD47 polypeptide or said extracellular domain of said adapter polypeptide is at least 70% identical to an extracellular domain of a CD47 polypeptide.

5. (canceled)

6. (canceled)

7. (canceled)

8. The method of claim 1, wherein said heterologous targeting domain comprises a tumor targeting domain.

9. The method of claim 8, wherein said tumor targeting domain is a CDX peptide.

10. The method of claim 8, wherein said tumor targeting domain is a CREKA peptide.

11. The method of claim 1 further comprising: nanoelectroporating a polynucleotide into said extracellular donor cell, wherein said polynucleotide encodes a ribonucleic acid (RNA) therapeutic.

12. The method of claim 11, wherein said ribonucleic acid (RNA) therapeutic is incorporated into extracellular vesicles released from said extracellular vesicle donor cell and said method further comprises collecting said extracellular vesicles released from said extracellular vesicle donor cell.

13. The method of claim 11, wherein said ribonucleic acid (RNA) therapeutic is a messenger RNA (mRNA), non-coding RNA, a microRNA, a shRNA, a siRNA, or a combination thereof.

14. (canceled)

15. The method of claim 11, wherein said RNA therapeutic is a cancer drug.

16. (canceled)

17. The method of claim 11, wherein said RNA therapeutic is fully intact or substantially intact messenger RNA.

18. The method of claim 17, wherein said RNA therapeutic comprises at least 5 copies of fully intact messenger RNA.

19. (canceled)

20. (canceled)

21. (canceled)

22. The method of claim 1, wherein said extracellular vesicle donor cell is selected from the group consisting of: primary cells, mouse embryonic fibroblasts (MEF), human embryonic fibroblasts (HEF), dendritic cells, mesenchymal stem cells, bone marrow-derived dendritic cells, bone marrow derived stromal cells, adipose stromal cells, endothelial cells, and immune cells.

23. (canceled)

24. (canceled)

25. The method of claim 1, wherein said extracellular vesicle is an exosome.

26. The method of claim 1, wherein said polynucleotide is nanoelectroporated into said extracellular vesicle donor cell via a nanochannel located on a biochip.

27. (canceled)

28. (canceled)

29. The method of claim 26, wherein said nanoelectroporation comprises an electric field.

30. The method of claim 29, wherein said electric field has an electric field strength from 1 volt/mm to 1000 volt/mm.

31. The method of claim 29, wherein said electric field comprises a plurality of pulses with pulse durations from 0.1 milliseconds/pulse to 100 millisecond/pulse.

32. (canceled)

33. (canceled)

34. A method of producing an extracellular vesicle, said method comprising:
a) nanoelectroporating a primary cell with at least one heterologous deoxyribonucleic acid (DNA) polynucleotide, thereby obtaining a primary cell comprising said heterologous DNA polynucleotide, wherein said heterologous DNA polynucleotide encodes a therapeutic ribonucleic acid (RNA) polynucleotide;
b) incubating said primary cell comprising said heterologous DNA polynucleotide under conditions to enable transcription of said heterologous DNA polynucleotide, thereby producing said therapeutic ribonucleic acid (RNA) polynucleotide, wherein said therapeutic ribonucleic acid (RNA) polynucleotide is incorporated into extracellular vesicles released from said primary cell; and
c) collecting said extracellular vesicles released from said primary cell, wherein said extracellular vesicles released from said primary cell comprise, on average, at least one copy of said therapeutic ribonucleic acid (RNA) polynucleotide.

35. (canceled)

36. (canceled)

37. (canceled)

38. (canceled)

39. (canceled)

40. A composition comprising an extracellular vesicle, said extracellular vesicle comprising:
a) an adapter polypeptide, wherein said adapter polypeptide comprises an extracellular domain, wherein said adapter polypeptide comprises a polypeptide sequence that is at least 70% identical to one of the following polypeptides: a CD47 extracellular domain, a CD47 transmembrane domain, CD63, CD81, CD82, CD47, CD315, heterotrimeric G protein, MEW class I, integrins, transferrin receptor (TFR2), LAMP1/2, heparan sulfate proteoglycans, EMMPRIN, ADAM10, GPI-anchored 5′nucleotidase, CD73, complement-binding protein CD55 and CD59, sonic hedgehog (SHH), TSPAN8, CD37, CD53, CD9, PECAM1, ERBB2, EPCAM, CD90, CD45, CD41, CD42a, Glycophorin A, CD14, MEW class II, CD3, Acetylcholinesterase/AChE-S, AChE-E, amyloid beta A4/APP, PTGFRN, and multidrug resistance-associated protein; and
b) a heterologous targeting polypeptide covalently attached to said extracellular domain of said adapter polypeptide, wherein said targeting polypeptide specifically binds to a cellular target.

41. The composition of claim 40, wherein said adapter polypeptide comprises a transmembrane domain that is at least 70% identical to a transmembrane domain of a CD47 polypeptide or an extracellular domain that is at least 70% identical to an extracellular domain of a CD47 polypeptide.

42.-159. (canceled)


SEQUENCE LISTING (117 sequences)

SEQ ID NO: 1 - protein, 323 aa, Artificial Sequence
  Synthetic Construct
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 2 - protein, 16 aa, Artificial Sequence
  Synthetic Construct
FKESWREARGTRIERG

SEQ ID NO: 3 - protein, 5 aa, Artificial Sequence
  Synthetic Construct
CREKA

SEQ ID NO: 4 - protein, 6 aa, Artificial Sequence
  Synthetic Construct
CKAAKN

SEQ ID NO: 5 - protein, 7 aa, Artificial Sequence
  Synthetic Construct
ARRPKLD

SEQ ID NO: 6 - protein, 7 aa, Artificial Sequence
  Synthetic Construct
SIGYPLP

SEQ ID NO: 7 - protein, 7 aa, Artificial Sequence
  Synthetic Construct
LSIPPKA

SEQ ID NO: 8 - protein, 7 aa, Artificial Sequence
  Synthetic Construct
FQTPPQL

SEQ ID NO: 9 - protein, 7 aa, Artificial Sequence
  Synthetic Construct
LTPATAI

SEQ ID NO: 10 - protein, 17 aa, Artificial Sequence
  Synthetic Construct
CNIWGVVLSWIGVFPEC

SEQ ID NO: 11 - protein, 5 aa, Artificial Sequence
  Synthetic Construct
NTTTH

SEQ ID NO: 12 - protein, 7 aa, Artificial Sequence
  Synthetic Construct
VHPKQHR

SEQ ID NO: 13 - protein, 18 aa, Artificial Sequence
  Synthetic Construct
CRKRLDRNCCRTLTVRKC

SEQ ID NO: 14 - protein, 9 aa, Artificial Sequence
  Synthetic Construct
CLWTVGGGC

SEQ ID NO: 15 - protein, 12 aa, Artificial Sequence
  Synthetic Construct
QPWLEQAYYSTF

SEQ ID NO: 16 - protein, 12 aa, Artificial Sequence
  Synthetic Construct
YPHIDSLGHWRR

SEQ ID NO: 17 - protein, 12 aa, Artificial Sequence
  Synthetic Construct
LLADTTHHRPWT

SEQ ID NO: 18 - protein, 12 aa, Artificial Sequence
  Synthetic Construct
SAHGTSTGVPWP

SEQ ID NO: 19 - protein, 12 aa, Artificial Sequence
  Synthetic Construct
VPWMEPAYQRFL

SEQ ID NO: 20 - protein, 12 aa, Artificial Sequence
  Synthetic Construct
TLPWLEESYWRP

SEQ ID NO: 21 - protein, 4 aa, Artificial Sequence
  Synthetic Construct
HWRR

SEQ ID NO: 22 - protein, 9 aa, Artificial Sequence
  Synthetic Construct
CSTSMLKAC

SEQ ID NO: 23 - protein, 7 aa, Artificial Sequence
  Synthetic Construct
DDTRHWG

SEQ ID NO: 24 - protein, 6 aa, Artificial Sequence
  Synthetic Construct
CARPAR

SEQ ID NO: 25 - protein, 6 aa, Artificial Sequence
  Synthetic Construct
CKRAVR

SEQ ID NO: 26 - protein, 9 aa, Artificial Sequence
  Synthetic Construct
CRSTRANPC

SEQ ID NO: 27 - protein, 9 aa, Artificial Sequence
  Synthetic Construct
CPKTRRVPC

SEQ ID NO: 28 - protein, 9 aa, Artificial Sequence
  Synthetic Construct
CSGMARTKC

SEQ ID NO: 29 - protein, 5 aa, Artificial Sequence
  Synthetic Construct
CRPPR

SEQ ID NO: 30 - protein, 9 aa, Artificial Sequence
  Synthetic Construct
CRVASVLPC

SEQ ID NO: 31 - protein, 9 aa, Artificial Sequence
  Synthetic Construct
SWCEPGWCR

SEQ ID NO: 32 - protein, 20 aa, Artificial Sequence
  Synthetic Construct
LSGTPERSGQAVKVKLKAIP

SEQ ID NO: 33 - protein, 18 aa, Artificial Sequence
  Synthetic Construct
CHVLWSTRCCVSNPRWKC

SEQ ID NO: 34 - protein, 7 aa, Artificial Sequence
  Synthetic Construct
LSALPRT

SEQ ID NO: 35 - protein, 7 aa, Artificial Sequence
  Synthetic Construct
CLPVASC

SEQ ID NO: 36 - protein, 9 aa, Artificial Sequence
  Synthetic Construct
  MISC_FEATURE (6)..(6): Arg or Met
  MISC_FEATURE (8)..(8): any amino acid
  MISC_FEATURE (9)..(9): Trp or Leu
ELRGDXAXX

SEQ ID NO: 37 - protein, 12 aa, Artificial Sequence
  Synthetic Construct
  MISC_FEATURE (3)..(3): Lys or Arg
  MISC_FEATURE (5)..(7): any amino acid
  MISC_FEATURE (8)..(8): Thr or Ser
  misc_feature (11)..(11): Xaa can be any naturally occurring amino acid
GVXGXXXXRDXR

SEQ ID NO: 38 - protein, 14 aa, Artificial Sequence
  Synthetic Construct
HITSLLSHTTHREP

SEQ ID NO: 39 - protein, 16 aa, Artificial Sequence
  Synthetic Construct
ANTPCGPYTHDCPVKR

SEQ ID NO: 40 - protein, 21 aa, Artificial Sequence
  Synthetic Construct
CGFELETCCGFECVRQCPERC

SEQ ID NO: 41 - protein, 30 aa, Artificial Sequence
  Synthetic Construct
QPFMQCLCLIYDASCRNVPPIFNDVYWIAF

SEQ ID NO: 42 - protein, 7 aa, Artificial Sequence
  Synthetic Construct
VNTANST

SEQ ID NO: 43 - protein, 9 aa, Artificial Sequence
  Synthetic Construct
CTSGTHPRC

SEQ ID NO: 44 - protein, 32 aa, Artificial Sequence
  Synthetic Construct
SGEWVIKEARGWKHWVFYSCCPTTPYLDITYH

SEQ ID NO: 45 - protein, 7 aa, Artificial Sequence
  Synthetic Construct
YSGKWGW

SEQ ID NO: 46 - protein, 36 aa, Artificial Sequence
  Synthetic Construct
LETTCASLCYPSYQCSYTMPHPPVVPPHPMTYSCQY

SEQ ID NO: 47 - protein, 7 aa, Artificial Sequence
  Synthetic Construct
YPRLLTP

SEQ ID NO: 48 - protein, 9 aa, Artificial Sequence
  Synthetic Construct
CSQSHPRHC

SEQ ID NO: 49 - protein, 9 aa, Artificial Sequence
  Synthetic Construct
CSKSSDYQC

SEQ ID NO: 50 - protein, 9 aa, Artificial Sequence
  Synthetic Construct
CKSTHPLSC

SEQ ID NO: 51 - protein, 10 aa, Artificial Sequence
  Synthetic Construct
CTGKSCLRVG

SEQ ID NO: 52 - protein, 12 aa, Artificial Sequence
  Synthetic Construct
SFKPSGLPAQSL

SEQ ID NO: 53 - protein, 9 aa, Artificial Sequence
  Synthetic Construct
CTANSSAQC

SEQ ID NO: 54 - protein, 9 aa, Artificial Sequence
  Synthetic Construct
CLSSRLDAC

SEQ ID NO: 55 - protein, 9 aa, Artificial Sequence
  Synthetic Construct
GHKAKGPRK

SEQ ID NO: 56 - protein, 7 aa, Artificial Sequence
  Synthetic Construct
HAIYPRH

SEQ ID NO: 57 - protein, 12 aa, Artificial Sequence
  Synthetic Construct
THRPPMWSPVWP

SEQ ID NO: 58 - protein, 13 aa, Artificial Sequence
  Synthetic Construct
HLNILSTLWKYRC

SEQ ID NO: 59 - protein, 7 aa, Artificial Sequence
  Synthetic Construct
CAGALCY

SEQ ID NO: 60 - protein, 9 aa, Artificial Sequence
  Synthetic Construct
CLEVSRKNC

SEQ ID NO: 61 - protein, 12 aa, Artificial Sequence
  Synthetic Construct
RPRTRLHTHRNR

SEQ ID NO: 62 - protein, 11 aa, Artificial Sequence
  Synthetic Construct
ACTTPHAWLCG

SEQ ID NO: 63 - protein, 14 aa, Artificial Sequence
  Synthetic Construct
GLAHSFSDFARDFV

SEQ ID NO: 64 - protein, 15 aa, Artificial Sequence
  Synthetic Construct
GYRPVHNIRGHWAPG

SEQ ID NO: 65 - protein, 12 aa, Artificial Sequence
  Synthetic Construct
TGNYKALHPHNG

SEQ ID NO: 66 - protein, 9 aa, Artificial Sequence
  Synthetic Construct
CRTIGPSVC

SEQ ID NO: 67 - protein, 9 aa, Artificial Sequence
  Synthetic Construct
CTSTSAPYC

SEQ ID NO: 68 - protein, 9 aa, Artificial Sequence
  Synthetic Construct
CSYTSSTMC

SEQ ID NO: 69 - protein, 8 aa, Artificial Sequence
  Synthetic Construct
CMPRLRGC

SEQ ID NO: 70 - protein, 12 aa, Artificial Sequence
  Synthetic Construct
TPSYDTYAAELR

SEQ ID NO: 71 - protein, 12 aa, Artificial Sequence
  Synthetic Construct
RLSSVDSDLSGC

SEQ ID NO: 72 - protein, 4 aa, Artificial Sequence
  Synthetic Construct
CAQK

SEQ ID NO: 73 - protein, 12 aa, Artificial Sequence
  Synthetic Construct
SGVYKVAYDWQH

SEQ ID NO: 74 - protein, 7 aa, Artificial Sequence
  Synthetic Construct
LMLPRAD

SEQ ID NO: 75 - protein, 9 aa, Artificial Sequence
  Synthetic Construct
CSCFRDVCC

SEQ ID NO: 76 - protein, 9 aa, Artificial Sequence
  Synthetic Construct
CRDVVSVIC

SEQ ID NO: 77 - protein, 13 aa, Artificial Sequence
  Synthetic Construct
CVALCREACGEGC

SEQ ID NO: 78 - protein, 7 aa, Artificial Sequence
  Synthetic Construct
GLSGGRS

SEQ ID NO: 79 - protein, 6 aa, Artificial Sequence
  Synthetic Construct
WYRGRL

SEQ ID NO: 80 - protein, 9 aa, Artificial Sequence
  Synthetic Construct
CPGPEGAGC

SEQ ID NO: 81 - protein, 14 aa, Artificial Sequence
  Synthetic Construct
SMSIARLVSFLEYR

SEQ ID NO: 82 - protein, 17 aa, Artificial Sequence
  Synthetic Construct
GPEDTSRAPENQQKTGC

SEQ ID NO: 83 - protein, 9 aa, Artificial Sequence
  Synthetic Construct
CKGGRAKDC

SEQ ID NO: 84 - protein, 9 aa, Artificial Sequence
  Synthetic Construct
CARSKNKDC

SEQ ID NO: 85 - protein, 9 aa, Artificial Sequence
  Synthetic Construct
CHAQGSAEC

SEQ ID NO: 86 - protein, 24 aa, Artificial Sequence
  Synthetic Construct
LEPRWGFGWWLKLSTHTTESRSMV

SEQ ID NO: 87 - protein, 14 aa, Artificial Sequence
  Synthetic Construct
ACSTEALRHCGGGS

SEQ ID NO: 88 - protein, 7 aa, Artificial Sequence
  Synthetic Construct
ASSLNIA

SEQ ID NO: 89 - protein, 15 aa, Artificial Sequence
  Synthetic Construct
DSLKSYWYLQKFSWR

SEQ ID NO: 90 - protein, 18 aa, Artificial Sequence
  Synthetic Construct
DWLKAFYDKVAEKLKEAF

SEQ ID NO: 91 - protein, 18 aa, Artificial Sequence
  Synthetic Construct
KSKTEYYNAWAVWERNAP

SEQ ID NO: 92 - protein, 21 aa, Artificial Sequence
  Synthetic Construct
GNGEQREMAVSRLRDCLDRQA

SEQ ID NO: 93 - protein, 22 aa, Artificial Sequence
  Synthetic Construct
HTPGNSNKWKHLQENKKGRPRR

SEQ ID NO: 94 - protein, 18 aa, Artificial Sequence
  Synthetic Construct
DWLKAFYDKVAEKLKEAF

SEQ ID NO: 95 - protein, 24 aa, Artificial Sequence
  Synthetic Construct
RRRRRRRRRGPLGLAGEEEEEEEE

SEQ ID NO: 96 - protein, 23 aa, Artificial Sequence
  Synthetic Construct
GAFSWGSLWSGIKNFGSTVKNYG

SEQ ID NO: 97 - protein, 5 aa, Artificial Sequence
  Synthetic Construct
RLRWR

SEQ ID NO: 98 - protein, 13 aa, Artificial Sequence
  Synthetic Construct
LGQQQPFPPQQPY

SEQ ID NO: 99 - protein, 16 aa, Artificial Sequence
  Synthetic Construct
ILGKLLSTAAGLLSNL

SEQ ID NO: 100 - protein, 19 aa, Artificial Sequence
  Synthetic Construct
TFFYGGSRGKRNNFKTEEY

SEQ ID NO: 101 - protein, 11 aa, Artificial Sequence
  Synthetic Construct
  MISC_FEATURE (11)..(11): any amino acid
LRKLRKRLLRX

SEQ ID NO: 102 - protein, 10 aa, Artificial Sequence
  Synthetic Construct
LRKLRKRLLR

SEQ ID NO: 103 - protein, 22 aa, Artificial Sequence
  Synthetic Construct
MVRRFLVTLRIRRACGPPRVRV

SEQ ID NO: 104 - protein, 16 aa, Artificial Sequence
  Synthetic Construct
FKESWREARGTRIERG

SEQ ID NO: 105 - protein, 16 aa, Artificial Sequence
  Synthetic Construct
FKESWREARGTRIERG

SEQ ID NO: 106 - DNA, 20 bp, Artificial Sequence
  Synthetic Construct
tggtgtctgaacctaagccc

SEQ ID NO: 107 - DNA, 20 bp, Artificial Sequence
  Synthetic Construct
gtccgagaactgacgttgct

SEQ ID NO: 108 - DNA, 19 bp, Artificial Sequence
  Synthetic Construct
cctatgaggaccagtctcc

SEQ ID NO: 109 - DNA, 19 bp, Artificial Sequence
  Synthetic Construct
gacatggctgtcactggat

SEQ ID NO: 110 - DNA, 18 bp, Artificial Sequence
  Synthetic Construct
gacacgccgacctcagac

SEQ ID NO: 111 - DNA, 19 bp, Artificial Sequence
  Synthetic Construct
gatccgcctctgcttgaat

SEQ ID NO: 112 - DNA, 20 bp, Artificial Sequence
  Synthetic Construct
gggaaattcaacggcacagt

SEQ ID NO: 113 - DNA, 19 bp, Artificial Sequence
  Synthetic Construct
agatggtgatgggcttccc

SEQ ID NO: 114 - DNA, 28 bp, Artificial Sequence
  Synthetic Construct
caagatgatgtttgaaactattccaatg

SEQ ID NO: 115 - DNA, 21 bp, Artificial Sequence
  Synthetic Construct
cctttagctggcagaccacaa

SEQ ID NO: 116 - DNA, 20 bp, Artificial Sequence
  Synthetic Construct
gacagtcagccgcatcttct

SEQ ID NO: 117 - DNA, 20 bp, Artificial Sequence
  Synthetic Construct
ttaaaagcagccctggtgac